(19) United States
(12) Patent Application Publication
(10) Pub. No.: US 2014/0158127 A1
(43) Pub. Date: Jun. 12, 2014

(54) NASAL CANNULA FOR DELIVERY OF AEROSOLIZED MEDICAMENTS

(71) Applicant: Parion Sciences, Inc., Durham, NC (US)

(72) Inventors: Paul BOUCHER, Cary, NC (US); Brian M. BUTTON, Hillsborough, NC (US); James B. FINK, San Mateo, CA (US); Anthony J. HICKEY, Chapel Hill, NC (US); Tomas NAVRATIL, Carrboro, NC (US); Stuart Robert ABERCROMBIE, Cambridge (GB); Michael Richard BURCHER, Cambridge (GB); Mark Jeffrey EDHOUSE, Cambridge (GB); Jamie Alan GREENWOOD, Royston (GB); Emma Jane HASLER, Suffolk (GB); Guy Conwyn Julian MOSELEY, Cambridge (GB); David William SMITH, Cambridge (GB); Donal Joseph TAYLOR, Cambridge (GB); Jonathan Hugh WILKINS, West Sussex (GB)

(73) Assignee: PARION SCIENCES, INC., Durham, NC (US)

(21) Appl. No.: 14/099,657

(22) Filed: Dec. 6, 2013

Related U.S. Application Data
(60) Provisional application No. 61/734,649, filed on Dec. 7, 2012.

(51) Int. Cl. A61M 16/06; A61M 16/14

(52) U.S. Cl. CPC A61M 16/0666; A61M 16/14
USPC 128/203.22

(57) ABSTRACT

An apparatus can include a nasal cannula assembly, which includes a face piece. The face piece includes a plenum portion and a nasal interface portion. The plenum portion is configured to be coupled to a supply line and defines a flow path configured to receive an aerosol flow from the supply line. The nasal interface portion includes a first delivery protrusion and a second delivery protrusion. The first delivery protrusion is configured to convey a first portion of the aerosol flow to a first nostril, and the second delivery protrusion is configured to deliver a second portion of the aerosol flow to a second nostril. The plenum portion includes a sidewall having a curved surface configured to redirect the second portion of the aerosol flow towards the second delivery protrusion. The sidewall is configured to isolate the flow path from a volume downstream from the second delivery protrusion.

### CROSS REFERENCE TO RELATED APPLICATIONS

This application is a claims benefit and priority to U.S. Provisional Application No. 61/734,649, filed Dec. 7, 2012, and entitled “Aerosol Delivery Systems and Related Methods,” the contents of which are hereby incorporated by reference herein in their entirety.

### BACKGROUND

Disclosed embodiments relate generally to devices, systems and methods for delivering aerosolized medicaments. More particularly, disclosed embodiments relate to nasal cannula assemblies and methods for delivery aerosolized medicaments transnasally into the lungs.

Aerosolized medicines are frequently used to treat individuals suffering from respiratory disease. For example, one known method for treating cystic fibrosis (CF) includes restoring hydration to the affected airway surfaces via the inhalation of a hypertonic osmolyte solution, which draws water onto the airway surface. Known methods often administer a seven percent (7%) hypertonic saline (HS) solution. Rehydration of the lubricant periciliary layer (PCL) of the airway surface facilitates mucus clearance (MC) and, therefore, the removal of inhaled infectious agents.

Known methods for delivering aerosolized medicaments include the inhalation of aerosols orally i.e., via an oral mouth piece or a spacer inserted into the patient's mouth. Other known methods for delivering aerosolized medicaments include transnasal delivery of the aerosolized medicament to the affected airways using a nasal cannula. Known nasal cannula assemblies, however, are commonly used for transnasal delivery of gases, for example, oxygen, to patients. Accordingly, although some known nasal cannula assemblies have been used for the transnasal delivery of aerosolized medicaments, they are not well-suited for the delivery of aerosolized medicaments. For example, such known nasal cannula assemblies are susceptible to “rainout” and “sputtering.” Rainout occurs due to agglomeration of the aerosolized medicaments into droplets within known nasal cannula assemblies due to gravitational or inertial sedimentation or condensation. The collected (or “rained-out”) aerosol often collects on an internal surface within the cannula, and is thus removed from the flow, which adversely impacts the delivery of the medicament. Sputtering occurs due to agglomeration of droplets of the aerosol into larger droplets, which exit the nasal cannula assembly (e.g., from the nasal prongs) or are otherwise separated from a surface on which the droplets (or rainout) collect. In addition to adversely impacting the delivery regimen, sputtering can produce significant patient discomfort.

More particularly, known cannula assemblies can include relatively long and/or narrow supply tubes through which the flow is communicated to the patient. When used for delivery of aerosolized medicaments, such supply tubes can be susceptible to sedimentation. For example, gravitational settlement can be exacerbated because of the length of the supply tube and/or nasal cannula assembly. Additionally, known nasal cannula assemblies can include bends, bifurcation joints or the like, that can be increase the occurrence of impaction, i.e., inertial rainout. Moreover, although the flow velocity of an aerosolized medicament can be increased to minimize the likelihood of gravitational sedimentation, such an increase can, however, exacerbate issues with inertial rainout.

In addition to the discomfort caused by rainout and sputtering, the flow performance of cannula assemblies can also impact the characteristics of the delivered aerosol flow. For example, the particle size of the aerosolized medicament and/or mass of the medicament (e.g., a salt such as NaCl, steroids, anti-biotics, anti-inflammatories, or any other medicament) communicated to the patient can vary based on the flow performance of the nasal cannula assembly. As another example, increased rainout and/or sputter can also result in a decrease in the delivery rate of the medicament.

Thus, a need exists for improved systems, devices and methods for delivering aerosolized medicaments transnasally to patients.

### SUMMARY

Embodiments described herein relate generally to devices, systems and methods for delivering aerosolized medicaments and more particularly, to nasal cannula assemblies for delivery of aerosolized medicaments transnasally into the lungs. In some embodiments, an apparatus can include a nasal cannula assembly, which includes a face piece. The face piece includes a plenum portion and a nasal interface portion. The plenum portion is configured to be coupled to a supply line and defines a flow path configured to receive an aerosol flow from the supply line. The nasal interface portion includes a first delivery protrusion and a second delivery protrusion. The first delivery protrusion is configured to convey a first portion of the aerosol flow to a first nostril, and the second delivery protrusion is configured to deliver a second portion of the aerosol flow to a second nostril. The plenum portion includes a sidewall which has a curved surface configured to redirect the second portion of the aerosol flow towards the second delivery protrusion. The sidewall is configured to isolate the flow path from a volume downstream from the second delivery protrusion.

### BRIEF DESCRIPTION OF THE DRAWINGS

FIG. 1 is a schematic block diagram of an aerosol delivery system according to an embodiment.

FIG. 2 shows a side cross-section view of a face piece, according to an embodiment.

FIG. 3 shows a side cross-section view of a face piece that has a supply line coupled thereto, according to an embodiment.

FIG. 4A shows a schematic illustration of a face piece that includes curved delivery protrusions, according to an embodiment. FIG. 4B shows a side cross-section view of the face piece of FIG. 4A taken along the line XX and FIG. 4C shows a side cross-section view of the face piece of FIG. 4A taken along the line YY.

FIG. 5 shows a side cross-section view of a face piece, according to an embodiment.

FIG. 6 shows a perspective view of a face piece, according to an embodiment.

FIG. 7 shows a front cross-section view of the face piece of FIG. 6.

FIG. 8 shows a back cross-section view of the face piece of FIG. 6.

FIG. 9 shows a cross-section of a first end portion of the face piece of FIG. 6

FIG. 10 shows a cross-section at the beginning of a plenum portion included in the face piece of FIG. 6.

FIG. 11 shows a cross-section taken along a first delivery protrusion included in the face piece of FIG. 6.

FIG. 12 shows a cross-section taken in the middle of the plenum portion included in the face piece of FIG. 6.

FIG. 13 shows a cross-section taken along a second delivery protrusion included in the face piece of FIG. 6.

FIG. 14 shows a side cross-section view of the face piece of FIG. 6 taken along line ZZ shown in FIG. 7.

FIG. 15 shows a perspective view of a face piece, according to an embodiment.

FIG. 16 shows a front cross-section view of the face piece of FIG. 15.

FIG. 17 shows a side cross-section view of a face piece that includes an early bifurcation, according to an embodiment.

FIG. 18 shows a side cross-section view of a face piece that includes a late bifurcation, according to an embodiment.

FIG. 19 shows a side cross-section view of a face piece that includes a plenum portion, according to an embodiment.

FIG. 20 shows a side cross-section view of a face piece that includes a plenum portion, according to an embodiment.

FIG. 21 shows a side cross-section view of a face piece that includes a plenum portion and an early bifurcation, according to an embodiment.

FIG. 22A shows a side cross-section view and FIG. 22B shows a top view of a face piece that includes non-circular delivery protrusions, according to an embodiment.

FIG. 23 shows a side cross-section view of a face piece that includes delivery protrusions configured to fit within the nares of a user, according to an embodiment.

FIG. 24 shows a side cross-section view of a face piece that includes flared delivery protrusions, according to an embodiment.

FIG. 25 shows a side cross-section view of a face piece that includes tapered delivery protrusions, according to an embodiment.

FIG. 26 shows a side cross-section view of a face piece that has one delivery protrusion blocked, according to an embodiment.

FIG. 27 shows a side cross-section view of a face piece that is devoid of any delivery protrusions, according to an embodiment.

FIG. 28 shows a side cross-section view of a face piece that is devoid of any delivery protrusions, according to an embodiment.

FIG. 29 shows a side cross-section view of a face piece having multiple fluid inlets, according to an embodiment.

FIG. 30 shows a side cross-section view of a face piece having multiple fluid inlets, according to an embodiment.

FIG. 31 shows a side cross-section view of a face piece that includes a grooved surface, according to an embodiment.

FIG. 32 shows a side cross-section view of a face piece that includes a rainout collection reservoir, according to an embodiment.

FIG. 33 shows a side cross-section view of a face piece that includes a membrane layer, according to an embodiment.

FIG. 34 shows a side cross-section view of a face piece that includes absorbent delivery protrusions, according to an embodiment.

FIG. 35 shows a side cross-section view of a face piece that includes a rainout wick, according to an embodiment.

FIG. 36 shows a magnetic coupling mechanism of a supply line to an aerosol preparation assembly, according to an embodiment.

FIG. 37A shows the magnetic coupling mechanism of FIG. 36 in a first configuration and FIG. 37B shows the magnetic coupling mechanism of FIG. 36 in a second configuration.

FIG. 38 shows a perspective view of a face piece mounting assembly, according to an embodiment.

FIG. 39 shows a perspective view of a face piece mounting assembly, according to an embodiment.

FIG. 40 shows a perspective view of a face piece mounting assembly, according to an embodiment.

FIG. 41 shows a perspective view of a face piece mounting assembly, according to an embodiment.

FIG. 42 shows a perspective view of a face piece mounting assembly, according to an embodiment.

FIG. 43 shows an enlarged view of the portion of the mounting assembly of FIG. 42 shown by the arrow 43.

FIG. 44 shows a side cross-section view of a portion of the mounting assembly of FIG. 42 taken along the line AA shown in FIG. 43.

FIG. 45 shows a perspective view of a face piece mounting assembly, according to an embodiment.

FIG. 46 shows a perspective view of a face piece mounting assembly, according to an embodiment.

FIG. 47A shows a perspective view of a mounting assembly for mounting a face piece to face of a user, according to an embodiment. FIG. 47B shows a side view of the mounting assembly of FIG. 47A.

FIG. 48A shows a perspective view of a mounting assembly for mounting a face piece to face of a user, according to an embodiment. FIG. 48B shows a perspective view of the face piece of FIG. 48A worn by a user.

FIGS. 49A-C show various configurations for routing a supply line relative to a user.

FIG. 50 shows an illustration of features configured to maintain a position of a supply line, according to an embodiment.

FIG. 51 is an image of a nasal cannula assembly according to an embodiment that includes face piece that has a unilateral flow path.

FIG. 52 shows the rainout performance data for the unilateral cannula assembly of FIG. 51 and two other cannula assemblies that have a bidirectional flow path, after 30 minutes of operation.

FIG. 53 shows the amount of NaCl delivered by the unilateral cannula assembly of FIG. 51 face piece and two other cannula assemblies that have a bidirectional flow path, after 30 minutes of operation.

FIG. 54 is a plot showing the predicted rainout as a function of face piece design.

FIG. 55A-E show photographs of various embodiments of face pieces.

FIG. 56 shows the amount of rainout as a percentage of nebulized mass of each of the face pieces of FIGS. 55A-E.

FIG. 57 shows a plot of the particle size of delivered aerosol by each of the face pieces of FIGS. 55A-E.

FIG. 58 shows a plot of the amount of NaCl delivered by each of the face pieces of FIGS. 55A-E.

FIG. 59 shows the computational fluid dynamic simulations on the face pieces shown in FIG. 55A and FIG. 55E to demonstrate flow balance between the delivery protrusions.

FIG. 60A shows a photograph of the rainout collected from the face piece of FIG. 55E, which includes curved delivery protrusions, and FIG. 60B shows a photograph of the rainout collected from a face piece that has straight delivery protrusions.

FIG. 61 shows a plot of the emitted particle size for the face piece of FIG. 55E over a delivery duration of 8 hours.

FIG. 62 shows a plot of the delivered NaCl for the face piece of FIG. 55E over a delivery duration of 8 hours

FIG. 63 shows a plot of the rainout and sputter performance for the face piece of FIG. 55E over a delivery duration of 8 hours.

### DETAILED DESCRIPTION

Embodiments of nasal cannula assemblies described herein are configured to substantially reduce rainout and sputtering such that aerosolized medicaments can be delivered transnasally to a patient for longer periods of time and with higher levels of comfort. Furthermore, embodiments of nasal cannula assemblies described herein can also provide better control over the particle size and mass of the aerosolized medicament communicated to a patient.

In some embodiments, an apparatus can include a nasal cannula assembly, which includes a face piece. The face piece includes a plenum portion and a nasal interface portion. The plenum portion is configured to be coupled to a supply line and defines a flow path configured to receive an aerosol flow from the supply line. The nasal interface portion includes a first delivery protrusion and a second delivery protrusion. The first delivery protrusion is configured to convey a first portion of the aerosol flow to a first nostril, and the second delivery protrusion is configured to deliver a second portion of the aerosol flow to a second nostril. The plenum portion includes a sidewall which has a curved surface configured to redirect the second portion of the aerosol flow towards the second delivery protrusion. The sidewall is configured to isolate the flow path from a volume downstream from the second delivery protrusion.

In some embodiments, an apparatus includes a nasal cannula assembly, which includes a face piece having a plenum portion and a nasal interface portion. The plenum portion has a side wall that defines a flow path configured to receive an aerosol flow. The nasal interface portion includes a delivery portion configured to convey at least a portion of the aerosol flow to a nostril. Furthermore, an inner surface of the side wall defining a portion of the flow path has a noncircular cross-sectional shape. In some embodiments, the noncircular cross-sectional shape has a length along a first axis of the cross-sectional shape and a width along a second axis of the cross-sectional shape such that the second axis is normal to the first axis and the length is greater than the width.

In some embodiments, an apparatus includes a nasal cannula assembly, which includes a face piece having a plenum portion and a nasal interface portion. The plenum portion has a side wall that defines a flow path configured to receive an aerosol flow. The nasal interface portion includes a first delivery protrusion and a second delivery protrusion. The first delivery protrusion is configured to convey a first portion of the aerosol flow to a first nostril and the second delivery protrusion is configured to convey a second portion of the aerosol flow to a second nostril. The flow path is characterized by a first cross-sectional flow area upstream from the first delivery protrusion and a second cross-sectional flow area between the first delivery protrusion and the second delivery protrusion. The second cross-sectional flow area is less than the first cross-sectional flow area. In some embodiments, the plenum portion includes a side wall having a curved surface that defines at least in part the second cross-sectional flow area. The curved surface is further configured to redirect the second portion of the aerosol flow towards the second delivery protrusion. In some embodiments, the side wall is configured to fluidically isolate the flow path from a volume downstream from the second delivery protrusion.

In some embodiments, an apparatus can include a face piece that includes a plenum portion and a nasal interface portion. The plenum portion defines a flow path and is configured to be fluidically coupled to a supply line to receive, within the flow path, an aerosol flow including aerosolized liquid particles having a volume median diameter (VMD) from about 0.5 μm to about 2.5 μm. The nasal interface portion includes a first delivery protrusion and a second delivery protrusion. The nasal cannula assembly is configured to convey a first portion of the aerosol flow to a first nostril via the first delivery protrusion and a second portion of the aerosol flow to a second nostril via the second delivery protrusion, such that an amount of the liquid particles deposited within the face piece is less than about ten percent of an amount of the liquid particles conveyed from the first delivery protrusion and the second delivery protrusion after thirty minutes. In some embodiments, the nasal cannula assembly can be configured such that an amount of the liquid particles deposited within the face piece is less than about two percent of an amount of the liquid particles conveyed from the first delivery protrusion and the second delivery protrusion after thirty minutes. In some embodiments, the nasal cannula assembly can be configured such that an amount of the liquid particles deposited within the face piece is less than about one percent of an amount of the liquid particles conveyed from the first delivery protrusion and the second delivery protrusion after thirty minutes.

In some embodiments, a method includes delivering an aerosolized osmolyte to a nasal cannula assembly. The nasal cannula assembly includes a supply tube and a face piece, the face piece including a plenum portion and a nasal interface portion. The nasal interface portion includes a first delivery protrusion and a second delivery protrusion. The plenum portion includes a side wall defining at least a portion of a flow path, the side wall configured to fluidically isolate the flow path from a volume downstream from the second delivery protrusion. The aerosolized osmolyte is delivered from the face piece via the flow path defined by the plenum portion such that a first portion of the aerosolized osmolyte is conveyed from the first delivery protrusion and a second portion of the aerosolized osmolyte is conveyed from the second delivery protrusion.

In some embodiments, the delivering the aerosolized osmolyte from the face piece is performed such that the rainout within the face piece is less than about ten percent by mass of the aerosolized osmolyte after a period of about thirty minutes. In some embodiments, the delivering the aerosolized osmolyte from the face piece is performed such that the amount of sputter conveyed from the face piece is less than about ten percent by mass of the aerosolized osmolyte after a period of about thirty minutes.

Subjects to be treated using the nasal cannula assemblies described herein include both human subjects and animal subjects (e.g., dog, cat, monkey, chimpanzee) for veterinary purposes. The subjects may be male or female and may be any suitable age, e.g., neonatal, infant, juvenile, adolescent, adult, or geriatric. In some embodiments, the subjects are preferably mammalian.

The terms “a” and “an,” when used to modify the ingredient of a composition, such as, active agent, buffering agent, and osmolyte, do not denote a limitation of quantity, but rather denote the presence of at least one of the referenced item. The term “or” or “and/or” is used as a function word to indicate that two words or expressions are to be taken together or individually. The terms “comprising,” “having,” “including,” and “containing” are to be construed as open-ended terms (i.e., meaning “including, but not limited to”). The endpoints of all ranges directed to the same component or property are inclusive and independently combinable.

“Osmolyte” active compounds as used herein refers to molecules or compounds that are osmotic ally active (i.e., are “osmolytes”). “Osmotically active” compounds may be membrane-impermeable (i.e., essentially non-absorbable) on the airway or pulmonary epithelial surface. Examples of “osmotically active” compounds are described in U.S. patent application Ser. No. 13/831,268 (also referred to as “the '268 application), filed Mar. 14, 2013 and entitled “Aerosol Delivery Systems, Compositions and Methods” the entire of contents of which are hereby incorporated by reference herein.

“Airway surface” and “pulmonary surface,” as used herein, include pulmonary airway surfaces such as the bronchi and bronchioles, alveolar surfaces, and nasal and sinus surfaces.

“Saline” as used herein refers to a solution comprised of, consisting of, or consisting essentially of sodium chloride in water. Saline can be hypertonic, isotonic, or hypotonic. In some embodiments, saline can comprise sodium chloride in an amount from about 0.1% to about 40% by weight, or any range therein, such as, but not limited to, about 0.1% to about 10%, about 0.5% to about 15%, about 1% to about 20%, about 5% to about 25%, about 10% to about 40%, or about 15% to about 35% by weight (in mg/100 mL). In certain embodiments, sodium chloride is included in a solution in an amount of about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40% by weight (in mg/100 mL), or any range therein.

“Hypertonic saline” as used herein refers to a solution comprised of, consisting of, or consisting essentially of greater than 0.9 wt % sodium chloride in water. In general, the sodium chloride is included in the solution in an amount of from about 0.9% to about 40% by weight, or any range therein, such as, but not limited to, about 1% to about 15%, about 5% to about 20%, about 5% to about 25%, about 10% to about 40%, or about 15% to about 35% by weight. In certain embodiments, sodium chloride is included in the solution in an amount of about 0.9%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40% by weight, or any range therein.

“Hypotonic saline” as used herein refers to a solution comprised of, consisting of, or consisting essentially of less than 0.9 wt % sodium chloride in water. In some embodiments, sodium chloride is included in the solution in an amount of about 0.9%, 0.8%, 0.7%, 0.6%, 0.5%, 0.4%, 0.3%, 0.2%, 0.1% by weight, or any range therein.

“Isotonic saline” as used herein refers to a solution comprised of, consisting of, or consisting essentially of 0.9 wt % sodium chloride in water.

According to some embodiments, saline (e.g., hypertonic saline) can include an excipient. An excipient can be a pharmaceutically acceptable excipient. “Pharmaceutically acceptable” as used herein means that the compound or composition is suitable for administration to a subject to achieve the treatments described herein, without unduly deleterious side effects in light of the severity of the disease and necessity of the treatment. Exemplary excipients include, but are not limited to, a buffer and/or a buffering agent (e.g., an anion, a cation, an organic compound, a salt, etc.). Exemplary buffers include, but are not limited to, carbonic acid/carbonate/bicarbonate-based buffers, disodium hydrogen phthalate/sodium dihydrogen orthophosphate-based buffers, tris(hydroxymethyl)aminomethane/hydrochloric acid-based buffers, barbitone sodium/hydrochloric acid-based buffers, and any combination thereof. Exemplary buffering agents include, but are not limited to, carbonic acid, carbonate, bicarbonate, disodium hydrogen phthalate, sodium dihydrogen orthophosphate, tris(hydroxymethyl)aminomethane, hydrochloric acid, barbitone sodium, dissolved CO₂ (e.g., CO₂ formulated at a pH of greater than 6.6), and any combination thereof. In certain embodiments, saline comprises a bicarbonate buffer excipient, such as a bicarbonate anion (HCO₃). In some embodiments, hypertonic saline can include sodium bicarbonate, sodium carbonate, carbonic acid, and/or dissolved CO₂ formulated at a pH of greater than 6.5. Additional ingredients can be included as desired depending upon the particular condition being treated, as discussed further below.

“Substantially dehydrate” as used herein with respect to airway epithelial cells refers to cellular dehydration sufficient to result in: (a) a loss of at least 5, 10, 15 or 20 percent of cell volume; (b) inhibition of the beat of cilia projecting from those cells by at least 20 or 40 percent; (c) a decrease in the ability of the dehydrated cells to donate water to, and thereby hydrate, their overlying airway surface liquid/mucus layer; and/or (d) produce pro-inflammatory states such as increased IL-8 secretion.

“Hydrate,” “hydration,” and grammatical variants thereof, as used herein, refers to bringing, placing, drawing and/or the like water onto an airway surface of a lung. In certain embodiments, hydration is enhanced by a method according to the embodiments described herein. Hydration reflects (a) an increase in the volume of airway surface liquid above the epithelial cells of at least about 1%, 5%, 10%, 15%, 20%, 100%, 100%, 500%, 1,000% or more, (b) dilution of mucins and/or mucus, and/or c) dilution of inflammatory materials.

The term “drug”, “active”, “medication,” “medicament,” or “active pharmaceutical ingredient,” or variants thereof, as used herein includes a pharmaceutically acceptable and therapeutically effective compound, pharmaceutically acceptable salts, stereoisomers and mixtures of stereoisomers, solvates, and/or esters thereof.

The term “derivative” as used herein refers to a chemical compound that is derived from or obtained from a parent compound and contains essential elements of the parent compound, but typically has one or more different functional groups. Such functional groups can be added to a parent compound, for example, to improve the molecule's solubility, absorption, biological half life, fluorescent properties, and the like, or to decrease the toxicity of the molecule, eliminate or attenuate any undesirable side effect of the molecule, and the like. It is to be understood that the term “derivative” encompasses a pharmaceutically acceptable salt, as described herein. An “active derivative” is a derivative that retains an activity recited herein (e.g., the ability to deliver a bioactive compound to a cell, cytotoxic activity).

The phrase “pharmaceutically acceptable salt(s),” as used herein, means those salts of the presently disclosed compounds that are safe and effective for use in a subject and that possess the desired biological activity.

Throughout the present specification, the terms “about” and/or “approximately” may be used in conjunction with numerical values and/or ranges. The term “about” is understood to mean those values near to a recited value. For example, “about 1200 [units]” may mean within ±25% of 1200 (e.g., from 30 to 50), within ±20%, ±15%, ±10%, ±9%, ±8%, ±7%, ±7%, ±5%, ±4%, ±3%, ±2%, ±1%, less than ±1%, or any other value or range of values therein or therebelow. Furthermore, the phrases “less than about [a value]” or “greater than about [a value]” should be understood in view of the definition of the term “about” provided herein. The terms “about” and “approximately” may be used interchangeably.

Throughout the present specification, numerical ranges are provided for certain quantities. It is to be understood that these ranges comprise all subranges therein. Thus, the range “from 50 to 80” includes all possible ranges therein (e.g., 51-79, 52-78, 53-77, 54-76, 55-75, 70-70, etc.). Furthermore, all values within a given range may be an endpoint for the range encompassed thereby (e.g., the range 50-80 includes the ranges with endpoints such as 55-80, 50-75, etc.).

As used herein, the term “volume median diameter” or “VMD” of an aerosol is the particle size diameter identified such that half of the volume of the aerosol particles is contained in particles with larger diameter than the VMD, and half of the volume of the aerosol particles is contained in particles with smaller diameter than the VMD.

As used herein, the term “rainout” refers to liquid (and/or a liquid/solid solution, suspension, emulsion, or colloid) from an aerosol that collects on a surface and/or is otherwise removed from the flow of the aerosol. Rainout can occur due to any suitable mechanism, such as inertial impaction, gravitational sedimentation, condensation, or Brownian motion on a surface. For example, “rainout” can refer to the agglomeration of aerosolized medicaments into droplets having a size greater than about 5 μm, greater than about 10 μm, greater than about 20 μm or greater than about 50 μm. The rained-out droplets can be collected and/or formed on an internal surface of the cannula assembly. The term “sputtering” refers to rainout that exits from a device (e.g., from the nasal prongs of a nasal cannula) or otherwise separates from the surface upon which the rainout is collected.

As used herein, the term “deposition efficiency” refers to the percentage of the delivered dose that is deposited into the area of interest. Thus, the deposition efficiency of a method and/or system for delivering an aerosolized medicament into the lungs is the amount by mass of the aerosol deposited into the lungs divided by the total amount of the aerosol delivered by the system to the nares.

As used herein, the term “circular” is understood to encompass any component or a portion of a component of any of the nasal cannula assemblies described herein, which has a circular cross-section such that a radial distance measured from a center line of the cross-section to an inner surface of the cross-section at any location is the same. The term “substantially” when used in conjunction with circular is intended to convey that the cross-section of any component or a portion of a component of any of the nasal cannula assemblies described herein is circular within manufacturing tolerances. Thus, a cross-section is considered substantially circular if a radial distance measured from a center line of the cross-section to an inner surface of the cross-section at any location varies by less than about 1%, less than about 2%, or less than about 5%.

Delivery Systems

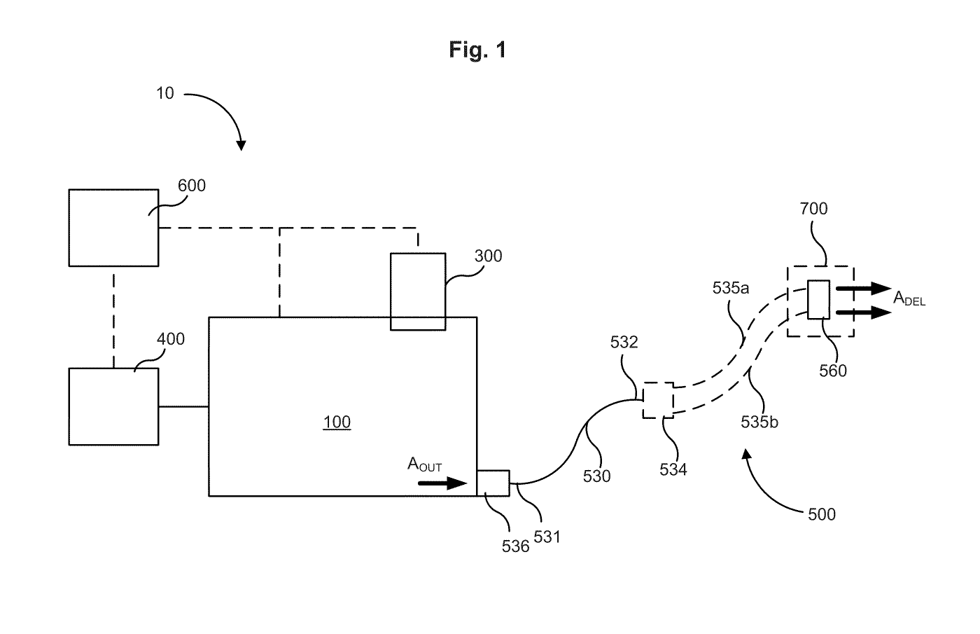

FIG. 1 is a schematic block diagram of an aerosol delivery system 10 according to an embodiment for delivering aerosolized medicaments (A_{DEL}) according to an embodiment. The aerosol delivery system 10 can be used to deliver any of the compositions according to any of the methods described herein. The system 10 includes an aerosol preparation assembly 100, a medicament cartridge 300, a source of gas 400, a nasal cannula assembly 500, and a controller 600. The system can optionally also include a mounting assembly 700 for mounting the nasal cannula assembly 500 (i.e., coupling the nasal cannula assembly to a patient, either directly or indirectly).

The aerosol preparation assembly 100 can be configured to produce aerosolized medicament A_{OUT} having specific characteristics, such as a desired particle size (e.g. VMD of between 1 and 3 microns) and/or size distribution, aerosol concentration, aerosol volume, medicament amount, flow rate, terminal velocity, and/or the like, which is delivered to the nasal cannula assembly 500. The aerosol preparation assembly 100 can be configured to generate both solid (i.e., fine solid particles in gas) and liquid (liquid droplets in gas) aerosols, depending on the medicament. The liquid can include, for example, a solid/liquid solution, a suspension, an emulsion, or a colloid. Any suitable mechanism can be employed for generating aerosol including, but not limited to an aerosol spray (similar to commonly used aerosol cans), an atomic nozzle (such as those based on the Venturi effect), any type of nebulizer suitable for medicament delivery (mechanical, electrical, a current jet nebulizer, an ultrasonic nebulizer, a vibrating mesh nebulizer etc.), an electrospray, a vibrating orifice aerosol generator (VOAG), droplet expulsion techniques (e.g. such as commonly used in ink jet printers), micro-scale nozzle membranes (e.g. such as can be generated via lithography, and particularly silicon wafer lithography), and/or the like. The disclosed embodiments can be configured to generate the aerosol independent of certain gas characteristics, such as, but not limited to, humidity, temperature, and/or the like. Accordingly, the aerosol preparation assembly 100, and the other aerosol preparation assemblies disclosed herein can receive inlet fluids (e.g., liquid medicament and inlet gas) having a wide range of input characteristics (e.g., droplet size, humidity, temperature), and can produce a repeatable outlet aerosol.

Generally, any suitable design of the aerosol preparation assembly 100 that permits generation and delivery of aerosolized medicament as described herein can be employed. For example, the aerosol preparation assembly 100 can be any of the aerosol preparation assemblies shown and described in the '268 application. In one example, the aerosol preparation assembly 100 can be configured to generate aerosol directly from liquid medicament and an entrainment gas. In another example, the aerosol preparation assembly 100 can be configured to generate an aerosol of the medicament prior to entrainment with the entrainment gas. The initial aerosol can be generated in a different stage of the aerosol preparation assembly 100 than another stage where the entrainment of the aerosol occurs. Further, any such stages of the aerosol preparation assembly 100 can be monolithically or separately constructed. In some embodiments, the aerosol preparation assembly 100 can be configured to modify one or more characteristics of the aerosolized medicament to better produce the specific characteristics associated with the indication to be treated, and can accordingly comprise any suitable component necessary for performing such function(s). For example, the aerosol preparation assembly 100 can be configured to increase the speed of the generated aerosol as can be desired to ensure delivery once the aerosol leaves the aerosol preparation assembly 100. Further, in some embodiments, the aerosol preparation assembly 100 can include a machine-readable label and/or electronic circuit system for communication with the controller 600 for monitoring, control, and/or generally modulating any of the functionality of the aerosol preparation assembly 100 as described herein.

The medicament cartridge 300 contains the medicament(s) to be aerosolized, and can be configured to be removably coupled and/or operatively coupled to the aerosol preparation assembly 100, and to deliver the medicament A. The medicament cartridge 300 can be configured to receive the medicament(s) at any suitable time, including at pre-filling and/or while coupled to the aerosol preparation assembly 100, and can be refillable or single use/disposable. The medicament cartridge 300 can be configured according to medicament-specific conditions to account for storage/delivery needs of the medicament. In some embodiments, the medicament cartridge 300 can include any keying feature that restricts the use of the medicament cartridge 300 to prespecified delivery systems, such as the aerosol preparation assembly 100. Additional components for handling and/or manipulating the medicament may be formed as part of the medicament cartridge 300, such as filters, for example. Further, in some embodiments, the medicament cartridge 300 can include a machine-readable label and/or electronic circuit system for communication with the controller 600 for monitoring the medicament levels in the medicament cartridge, for controlling access/delivery of the medicament, and/or the like.

The gas source 400 can provide a gas flow in a manner appropriate for the aerosol preparation assembly 100 (i.e., to produce aerosolized medicaments (A_{DEL}) having the desired characteristics). In other words, the gas source 400 can, in some embodiments, be tuned to the specifications of input requirements of the aerosol preparation assembly 100. For example, in some embodiments, the gas source 400 can be operated to produce steady, laminar flow, while in other embodiments, the gas source can produce flow having periodic changes in local velocity, pressure, and/or any suitable flow parameter. Although shown here as a single gas source 400 for simplicity, it is understood that in some embodiments, a system can include multiple gas sources operable to deliver one or more gases, each operating in a similar manner as described here. The gas source 400 can be of any suitable form, such as a pump, a hospital supply system, a gas tank (e.g. most medical gas supplies), and/or the like. In some embodiments, the gas source need not be humidified and/or otherwise controlled for humidity, temperature, and/or the like. Additional components for handling and/or manipulating the gas may be formed as part of the gas source 400, such as pumps, connecting lines, compliance chambers, filters, valves, regulators, pressure gauges, and/or the like. Further, in some embodiments, the gas source 400 can include a machine-readable label and/or electronic circuit system (e.g. a hydraulic control system) for communication with the controller 600 for monitoring gas levels in the gas source, for controlling access/delivery of the gas, for controlling gas flow parameters, and/or the like. Examples of aerosol delivery systems are described in the '268 application.

The nasal cannula assembly 500 is configured to receive the aerosolized medicament A_{OUT} from the aerosol preparation assembly 100 and deliver the aerosolized medicament A_{DEL} from the aerosol preparation assembly 100 to nares of a patient. In some embodiments, the nasal cannula assembly 500 can be configured to be removably coupled and/or operatively coupled to the aerosol preparation assembly 100. As shown in FIG. 1, in some embodiments, the nasal cannula assembly includes a supply line 530 and face piece 560. A proximal end 531 of the supply line is coupled with the aerosol preparation assembly 100 and is configured to receive an outlet aerosol A_{OUT} from the aerosol preparation assembly 100. The supply line 530 includes a coupling mechanism 536 configured to removably couple the supply line 530 to the aerosol preparation assembly 100. The coupling mechanism 536 can include, for example magnetic connectors, a friction fit connector, clamp connector, luer connector, or any other suitable coupling mechanism.

The supply line 530 can be of a certain minimum rigidity to prevent excessive bending that could, in turn, affect flow characteristics detrimentally. Said another way, in some embodiments, the supply line 530 and any of the supply lines described herein, can be configured to include limited bends and/or include bend radii above a particular value (e.g., within the range of 5 degrees to about 60 degrees, for example about 10 degrees, about 15 degrees, about 20 degrees, about 25 degrees, about 30 degrees about, 35 degrees, about 40 degrees, about 45 degrees, about 50 degrees, about 55 degrees or about 60 degrees, inclusive of all values therebetween) to minimize, reduce, and/or otherwise eliminate impaction and/or inertial sedimentation. In some embodiments, additional structures may be formed within the supply line 530, such as one way valves that prevent condensed liquids/particles from flowing into the patient's nostrils, but permit backflow of the condensed liquids/particles into the aerosol preparation assembly 100, for example. In some embodiments, one or more filtering structures that change the particle size distribution of the aerosolized medicament can also be present in the supply line 530. In some embodiments, the supply line 530 can include ribs or fins disposed on an interior surface of the supply line 530 to add stiffness and/or limit bending. In other embodiments, the supply line 530 can be substantially free of internal structures, such as ridges, support structures and/or internal channels, to minimize and/or reduce the internal surface area and/or ratio between the surface area and the flow area.

A distal end 532 of the supply line 530 is coupled to the face piece 560. In some embodiments, the distal end 532 of the supply line can be coupled to a bifurcation piece 534, for example a Y joint, a T-connector, or any other suitable connector that has a first fluidic inlet (not shown) coupled to the distal end 532 of the supply line and configured to receive the outlet aerosol A_{OUT}. The bifurcation joint 534 can further include a first fluidic outlet (not shown) coupled to a first face piece tube 535a and a second fluidic outlet (not shown) coupled to a second face piece tube 535b. In some embodiments, the bifurcation joint 534 can be configured to deliver a first portion of the outlet aerosol A_{OUT} to the first face piece tube 535a via the first fluidic outlet and a second portion of the outlet aerosol to the second face piece tube 535b via the second fluidic outlet. In some embodiments, the second fluidic outlet included in the bifurcation joint 534 can be blocked (e.g., blocked using a filling material, a valve or the like) or the second face piece tube 535b can be a solid tube that does not define a lumen, such that substantially all of the outlet aerosol A_{OUT} is communicated to the first face piece tube 535a via the first fluidic outlet. In such embodiments, the second face piece tube 535b can serve as a mounting tube for the face piece 560, such that aerosol is delivered to the face piece 560 only through the first face piece tube 535a (i.e., unilateral delivery).

In some embodiments, the supply line 530 can include a first supply line and a second supply line (not shown), each of the first supply line and the second supply line coupled to the aerosol preparation assembly 100 and configured to receive at least a portion of the outlet aerosol A_{OUT}. Each of the first supply line and the second supply line can also be coupled to the face piece 560 to deliver the portions of the outlet aerosol to the face piece 560. In some embodiments, the distal end 532 of each the first supply line and the second supply line can include nasal interface portions configured to interface with the nares of a subject (e.g., a patient) such that the aerosol delivery system 10 does not include a face piece.

The face piece 560 can be removably coupled and/or operatively coupled to the distal end 532 the supply line 530, either directly or, as shown in FIG. 1, via the bifurcation piece 534 through the face piece tubes 535a and/or 535b. The face piece 560 is appropriately sized and configured, as set forth herein, to provide the desired delivery characteristics of the aerosolized medicament. As generally discussed above for the tube 530, the face piece 560 can constitute filters, valves, and/or the like of the types shown and described herein for modifying flow characteristics and or the aerosolized medicament. In some embodiments, the face piece can include a plenum portion (not shown) configured to define flow path for receiving the outlet aerosol A_{OUT} from the aerosol preparation assembly 100 via the supply line 530. In some embodiments, the plenum portion can include a single fluidic inlet configured to be fluidically coupled to the supply line 530 or a face piece tube (e.g., any one of the face piece tubes 535a or 535b). The face piece 560 also includes a nasal interface portion (not shown) configured to deliver an aerosol A_{DEL} intranasally to a subject. In some embodiments, the nasal interface portion can include a first delivery protrusion configured to convey a first portion of the aerosol flow A_{DEL} to a first nostril, and a second delivery protrusion configured to convey a second portion of the aerosol flow A_{DEL} to a second nostril.

In some embodiments, the face piece 560 can include a mounting portion configured to receive a mounting member, for example the face piece tube 535b for mounting the face piece 560 in proximity of the nostrils of a subject. In some embodiments, the face piece 560 can be further configured for ease and comfort of use, by including features such as claspers, adhesive pads, and/or the like to hold the face piece 560 in position on or adjacent the nose of the patient.

In some embodiments, the aerosol delivery system 10 can optionally include a mounting assembly 700 configured to mount the nasal cannula assembly 500 either directly or indirectly to at least a portion of the face of a patient. For example, the mounting assembly 700 can include members or features that can be coupled to the supply line 530, the first face piece tube 535a the second face piece tube 535b and/or the face piece 560. The mounting assembly 700 can include adhesive members, braces, pads, cushions (e.g., ear cushions), head gears, mounting tubes that do not define a flow path, and/or any other mounting members. Any or all features included in the mounting assembly 700 can be ergonomically designed so that the comfort of a patient is not adversely affected even if the patient wears the mounting assembly 700 for long periods of time (e.g., greater than 30 minutes, 1 hour, 2 hours, 4 hours and up to 8 hours). The mounting assembly 700 can be configured to be worn by a patient on at least a portion of the face or head of the patient, for example, around the ears of the patient, on the head of the patient, stuck to a forehead with an adhesive, worn as an eye mask, and/or a head brace, such that the face piece 560 is disposed in proximity to the nares of the patient.

The controller 600 can be configured for monitoring, controlling, and/or modulating any of the functionality of the aerosol preparation assembly 100, the medicament cartridge 300, the gas source 400, and/or any other component associated with the system 10. In some embodiments, the controller 600 can include at least a processor and a memory. In some embodiments, the controller 600 can receive signal inputs and produce outputs to control and/or operate the system 10, as described herein.

The memory can be any suitable computer memory. For example, the memory can be random-access memory (RAM), read-only memory (ROM), flash memory, erasable programmable read-only memory (EPROM), electrically erasable programmable read-only memory (EEPROM), and/or other suitable memory. In some embodiments, the memory can be configured to store code representing processor instructions for execution by the processor and/or store data received from any device(s) operatively coupled to the processor.

The processor can be any suitable processor capable of executing computer instructions. Each module in the processor can be any combination of hardware-based module (e.g., a field-programmable gate array (FPGA), an application specific integrated circuit (ASIC), a digital signal processor (DSP) and/or software-based module (e.g., a module of computer code stored in memory and/or executed at the processor) configured to execute a specific function of the system 10. The processor can be a microcontroller, a FPGA, an ASIC, or any other suitable processor configured to run and/or execute the modules. The processor and modules of the processor can be configured to collectively execute the methods described herein, and/or to implements the apparatuses described herein.

In use, the aerosol preparation assembly 100 can produce an outlet aerosol A_{OUT} having the desired particle size distribution to accommodate the methods of delivery and/or treatment described herein. In some embodiments, the aerosol includes a plurality of liquid particles (e.g., an aqueous dug) entrained in a stream of a gas (e.g., air or oxygen). In some embodiments, the aerosol preparation assembly 100 can be configured to produce a flow rate of the outlet aerosol A_{OUT} to the nasal cannula assembly 500 of about 0.1 L/min to about 1 L/min, about 1 L/min to about 2 L/min, about 2 L/min to about 3 L/min, about 3 L/min to about 4 L/min, about 1 L/min to about 3 L/min, or about 1.5 L/min to about 2.5 L/min, inclusive of all ranges therebetween. In some embodiments, the plurality of liquid particles included in the outlet aerosol A_{OUT} can have a liquid flow rate of less than about 200 μl/min and a gas flow rate in the range of about 1 L/min to about 3 L/min. The amount of liquid particles in the outlet aerosol A_{OUT} (e.g., the liquid flow rate) can be of any suitable value to achieve the desired therapeutic benefits as described herein. In some embodiments, the liquid flow rate of the plurality of liquid particles included in the outlet aerosol A_{OUT} can be about 0.1 μl/min, about 0.2 μl/min, about 0.5 μl/min, about 1 μl/min, about 1.5 μl/min, about 2 μl/min, about 2.5 μl/min, about 3 μl/min, about 4 μl/min, about 5 μl/min, about 6 μl/min, about 7 μl/min, about 8 μl/min, about 9 μl/min, about 10 μl/min, about 20 μl/min, about 30 μl/min, about 40 μl/min, about 50 μl/min, about 60 μl/min, about 70 μl/min, about 80 μl/min, about 90 μl/min, about 100 μl/min, about 120 μl/min, about 140 μl/min, about 160 μl/min, about 180 μl/min, or about 200 μl/min, and all values in between. The aerosol preparation assembly 100 can be configured such that a VMD of the outlet aerosol A_{OUT} is less than a VMD of an inlet aerosol conveyed into the aerosol preparation assembly 100. For example, in some embodiments, the VMD of the inlet aerosol can be about 3 microns, about 4 microns, about 5 microns, about 6 microns, about 7 microns, about 8 microns, about 9 microns, about 10 microns, including all values therebetween, and the VMD of the outlet aerosol A_{OUT} can be about 0.5 microns, about 1 micron, about 1.1 microns, about 1.2 microns, about 1.3 microns, about 1.4 microns, about 1.5 microns, about 1.6 microns, about 1.7 microns, about 1.8 microns, about 1.9 microns, about 2 microns, about 2.5 microns, including all value therebetween. In some embodiments, the percentage of aerosol particles above 4 μm included in the outlet aerosol A_{OUT} can be less than about 5%, less than about 4%, less than about 3%, less than about 2%, or less than about 1% of the total particle volume.

The cannula assembly 500 and/or the face piece 560 is configured to minimize, reduce, and/or otherwise eliminate rainout and/or sputtering such that, for example, substantially no rainout and/or sputter is emitted from the face piece 560 (e.g., from the first delivery protrusion and the second delivery protrusion included in the face piece 560) after about 30 minutes of operation. In some embodiments, the face piece 560 can include only one fluid inlet (e.g., the fluid inlet included in the plenum portion) for coupling to the supply line 530 or face piece tube (e.g., the face piece tube 535a). An assembly having single supply line 530 (i.e., that is devoid of two separate face piece tubes 535a, 535b) will result in a unidirectional aerosol flow, which can minimize, reduce and/or abate impaction and thereby, reduce rainout and/or sputtering. In some embodiments, the face piece 560 and any of the face pieces described herein (e.g., including the face piece 5560) can be configured such that the amount of liquid particles deposited within face piece 560 (or the face piece 5560) is less than about 10% of an amount of liquid communicated in the delivered aerosol A_{DEL} by the first delivery protrusion and/or the second delivery protrusion after thirty minutes of operation. For example, the amount of liquid can be less than about 9%, less than about 8%, less than about 7%, less than about 6%, less than about 5%, less than about 4%, less than about 3% less than about 2%, less than about 1%, less than about 0.5%, less than about 0.4%, less than about 0.3%, less than about 0.2%, or less than about 0.1% after thirty minutes of operation. In some embodiments, the face piece 560, and any of the face pieces described herein (e.g., including the face piece 5560), can be configured such that no sputter is emitted from the first delivery protrusion and the second delivery protrusion after thirty minutes of operation. In some embodiments, the face piece 560, and any of the face pieces described herein (e.g., including the face piece 5560), can be configured such that a flow rate of the first portion of the aerosol flow delivered to the first delivery protrusion is within about 10% of the flow rate of the second portion of the aerosol flow delivered to the second delivery protrusion.

In some embodiments, the particle size distribution of the delivered aerosol A_{DEL} is controlled to minimize, reduce and/or abate the amount of aerosol impaction in the nasal passages (i.e., the filtering of particles via the nose) by minimizing or substantially eliminating impaction and/or sedimentation of the aerosol, as the aerosol is conveyed therethrough. More particularly, it is known that the nose is an effective filter for particles that are greater than approximately 2-3 μm. Thus, transnasal delivery of aerosols having a VMD of, for example, 6 μm, will result in low rates of deposition into the lower airways. The aerosol preparation assembly 100 and the nasal cannula assembly 500 that includes the face piece 560 are thus collectively configured to deliver an aerosol A_{DEL} to the nares of the patient having a desired particle size (e.g., a desired VMD of less than about 3 μm). Said another way, while the aerosol preparation assembly 100 can produce an outlet aerosol A_{OUT} having a desired particle size, the particle size of the delivered aerosol (A_{DEL}) is also affected by the cannula assembly coupled to the aerosol preparation assembly 100. The nasal cannula assembly 500, and any of the nasal cannula assemblies described herein, is thereby configured to deliver the desired aerosol flow while maintaining and/or enhancing the characteristics of the delivered aerosol A_{DEL}. In some embodiments, the delivered aerosol A_{DEL} can include an aerosol of liquid particles that have a VMD in the range of about 0.5 μm to about 5 μm, for example, about 1 μm, about 2 μm, about 3 μm, about 4 μm, or about 2.5 μm inclusive of all ranges therebetween, to accommodate a particular method of treatment and/or to deliver a medicament having a particular composition. The aerosol can include any suitable medicament that can be intranasally delivered to a patient as described herein.

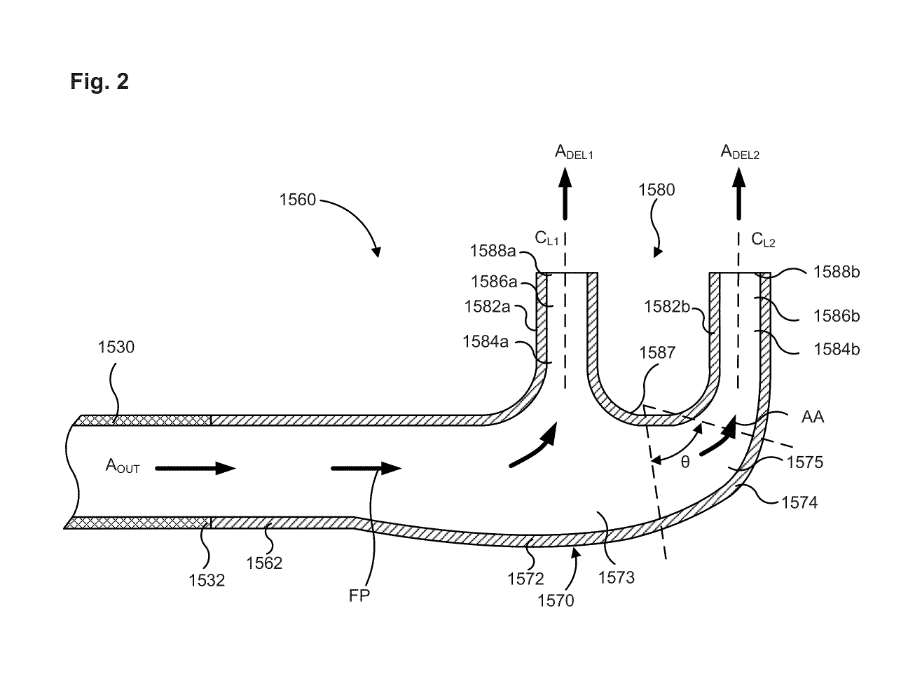

Although shown in FIG. 1 as receiving flow from two face piece tubes, in some embodiments, a nasal cannula assembly can include a face piece that is configured to receive an aerosol flow from only one direction. For example, FIG. 2 shows a face piece 1560 that includes a plenum portion 1570 and a nasal interface portion 1580. The face piece 1560 can be included in any of the nasal cannula assemblies shown and described herein, such as, for example the nasal cannula assembly 500, and can be used to accomplish any of the methods described herein. The nasal interface portion 1580 includes a first delivery protrusion 1582a and a second delivery protrusion 1582b. The first delivery protrusion 1582a is configured to deliver a first portion of the aerosol flow A_{DEL1} towards a first nostril and the second delivery protrusion 1582b is configured to convey a second portion of the aerosol flow A_{DEL2} towards a second nostril, as described herein.

The face piece 1560 includes a first end portion 1562 that is fluidically coupled to the distal end of a supply line 1530 and is configured to receive an outlet aerosol A_{OUT} from an aerosol preparation assembly, for example the aerosol preparation assembly 100, via the supply line 1530. The plenum portion 1570 includes a first side wall 1572 that has an inner surface 1573 defining at least a portion of a flow path FP. The flow path FP of the plenum portion 1570 is substantially continuous with and/or fluidically coupled to the first end portion 1562, such that flow path FP of the plenum portion 1570 is configured to receive the outlet aerosol A_{OUT} from the supply line 1530. The plenum portion 1570 also includes an end side wall 1574 having a curved surface 1575 configured to redirect the second portion of the aerosol flow towards the second delivery protrusion 1582b, as shown by the arrow AA. Moreover, the end side wall 1574 is configured to fluidically isolate the flow path from a volume downstream from the second delivery protrusion 1582b.

Thus, the outlet aerosol A_{OUT} is communicated into the flow path FP defined by the plenum portion 1570 through a single fluidic inlet using a single supply line 1530, such that the aerosol flow within the plenum portion 1570 is unidirectional. The unidirectional arrangement employs a single supply line 1530 (as opposed to multiple supply lines and/or face piece tubes), which reduces the surface area of the flow path FP (as compared to the flow area). The reduced surface area reduces the likelihood of sedimentation of aerosol droplets, which can increase rainout and/or sputtering. Furthermore, the unidirectional arrangement includes a single inlet, thereby reducing and/or eliminating opposing air flows within the face piece, which can yield to impaction and sedimentation of the aerosol droplets.

In some embodiments, the face piece 1560 and/or the end side wall 1574 can be configured to limit recirculation of the second portion of the aerosol flow within the flow path FP. More particularly, the face piece 1560 and/or the end side wall 1574 can be configured to limit recirculation at a location downstream of the second delivery protrusion 1582b. As used herein, “recirculation” means a change in flow direction of greater than about 90 degrees; i.e., at least a partially “doubling back” of the flow. Accordingly, in some embodiments the face piece 1560 and/or the end side wall 1574 are configured to limit and/or prevent a portion of the aerosol flow from forming eddies and/or other flow patterns that cause recirculation back into the flow path defined by the inner surface 1573 of the first side wall 1572. Thus, in some embodiments, the curved surface 1575 of the end side wall can be configured to define an angle of curvature θ of less than about 90 degrees, for example less than about 85 degrees, less than about 80 degrees, less than about 75 degrees, or less than about 70 degrees. In addition to being configured to limit recirculation, in some embodiments, the flow path FP defined by the plenum portion 1570 can include a first cross-sectional flow area upstream from the first delivery protrusion 1582a and a second flow cross-sectional area between the first delivery protrusion 1582a and the second delivery protrusion 1582b, such that the second cross-sectional flow area is less than the first cross-sectional flow area. Similarly stated, in some embodiments, at least a portion of the end side wall 1574 can be configured to define a flow restriction within the flow path, for example the smaller cross-sectional flow area of the curved surface 1575 can define the flow restriction.

The first delivery protrusion 1582a includes an inner surface 1584a that defines a first flow path 1586a. Similarly, the second delivery protrusion 1582b also includes an inner surface 1584b that defines a second flow path 1586b. The first delivery protrusion 1582a is configured to deliver a first portion of the aerosol flow A_{DEL1} towards a first nostril (e.g., the nostril of a patient) via the outlet 1588a, and the second delivery protrusion is configured to convey a second portion of the aerosol flow A_{DEL2} towards a second nostril via the outlet 1588b. As shown in FIG. 2, center line C_{L1} of the first nasal flow path 1586a of the first delivery protrusion 1582a and a center line C_{L2} of the second nasal flow path 1586b of the second delivery protrusion 1582b can be substantially straight. In other embodiments, the center line C_{L1} of the first nasal flow path 1586a defined by the first delivery protrusion 1582a and/or the center line C_{L2} of the second nasal flow path 1586b defined by the second delivery protrusion 1582a can be curved. In such embodiments, the curvature of the curved nasal flow paths can, for example, be configured to ergonomically interface with the nostrils of a patient, and/or alleviate rainout and/or sputtering. In some embodiments, the curvature can be less than about 30 degrees. In some embodiments, the curved surface 1575 of the plenum portion 1570 and the second nasal flow path can form a continuous boundary between the flow path of the plenum portion 1570 and the second nasal flow path 1586b.

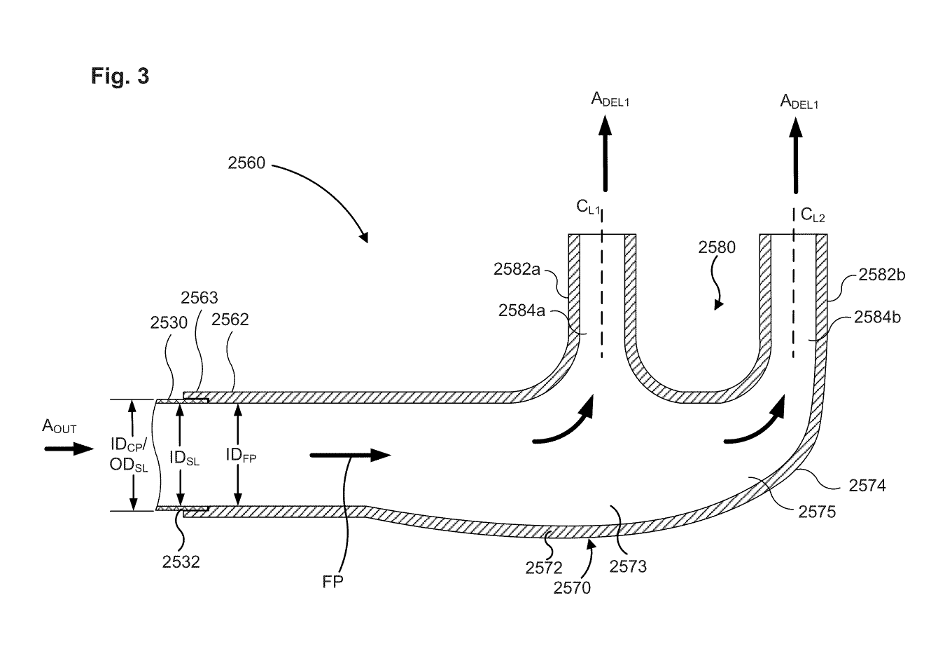

In some embodiments, the end portion 1562 of the face piece 1560 can include a connection portion configured to be coupleable to the supply line 1530 such that an inner surface of the supply line 1530 and an inner surface defining the flow path of the plenum portion form a substantially continuous surface. For example, FIG. 3 shows a face piece 2560 that includes a plenum portion 2570 and a nasal interface portion 2580. The face piece 2560 can be included in any of the nasal cannula assemblies shown and described herein, such as, for example the nasal cannula assembly 500, and can be used to accomplish any of the methods described herein. The nasal interface portion 2580 includes a first delivery protrusion 2582a and a second delivery protrusion 2582b. The first delivery protrusion 2582a is configured to deliver a first portion of the aerosol flow A_{DEL1} towards a first nostril and the second delivery protrusion 2582b is configured to convey a second portion of the aerosol flow A_{DEL2} towards a second nostril, as described herein. The plenum portion 2570 and the nasal interface portion 2580 can be substantially similar to the plenum portion 1570 and the nasal interface portion 1580 described with respect to the face piece 1560 and therefore, are not described in further detail herein.

The face piece 2560 includes an end portion 2562 which defines a connection portion 2563 configured to be coupleable to a distal end 2532 of a supply line 2530. The supply line 2530 and the connection portion 2563 are configured such that an outer cross-section, size and/or diameter OD_{SL} of the supply line 2530 is substantially equal to an inner cross-section, size and/or diameter ID_{CP} of the connection portion 2563. Furthermore, an inner cross-section, size and/or diameter ID_{SL} of the supply line 2530 can be substantially equal to the inner cross-section, size and/or diameter ID_{FP} of the end portion 2562 of the face piece 2560. This ensures that when the supply line 2530 is coupled to the face piece 2560, an inner surface of the supply line 2530 and an inner surface 2573 defining a flow path FP of the plenum portion form a substantially continuous surface. In such a configuration, the outlet aerosol A_{OUT} entering into the face piece 2560 from the supply line 2530 does not encounter any flat surface, sharp bends, steps, or corners, which can lead to localized flow recirculation, eddies, as well as areas for collection and/or impaction of the aerosol. Thus, this arrangement reduces impaction and reduces rainout and/or sputtering. The supply line 2530 can be coupled to the connection portion 2563 using any suitable means, for example, friction fit into the connection portion 2563, connected via luer lock, using an adhesive, snap-fit, or any other suitable coupling mechanism.

As shown in FIG. 3, the face piece 2560 includes only one connection portion 2563 disposed in the end portion 2562 and configured to be coupled to the supply line 2530. In some embodiments, the face piece 2560 (or the face piece 1560) can also include a second end portion (not shown) disposed at a second end of the face piece 2560 (e.g., disposed opposite the first end portion 2562 along a longitudinal axis defined by the flow path of the plenum portion 2570). The second end portion can include a second connection portion that is fluidically isolated from the flow path defined by the plenum portion and is configured to be coupleable to a mounting member. In such embodiments, the supply line 2530 and the mounting member can be configured to mount the face piece 2560 in proximity of the nostrils of a patient, as described herein.

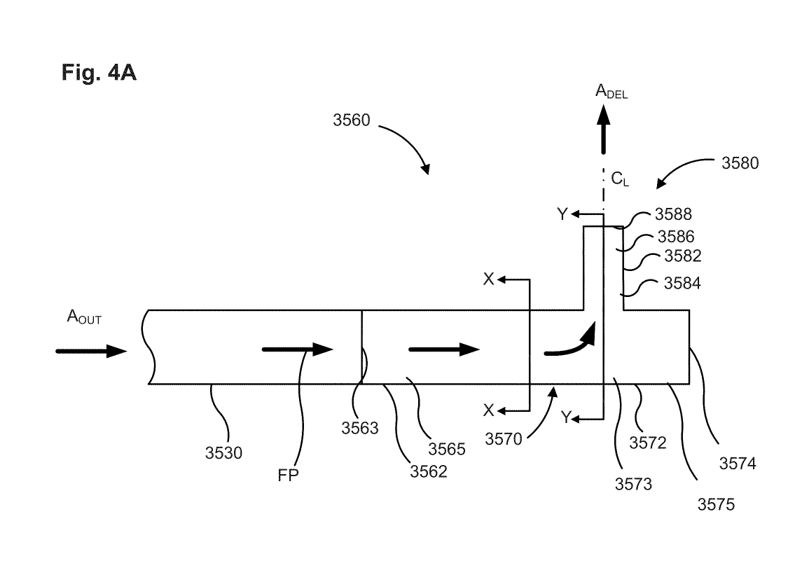

In some embodiments, a nasal cannula assembly can include face piece that defines a non-circular flow path. Similarly stated, in some embodiments, at least a portion of a flow area defined by a face piece can have a non-circular cross-sectional shape. Referring now to FIG. 4A-C, a face piece 3560 is shown that includes a plenum portion 3570 and a nasal interface portion 3580. The face piece 3560 can be included in any of the nasal cannula assemblies shown and described herein, such as, for example the nasal cannula assembly 500, and can be used to accomplish any of the methods described herein. The nasal interface portion 3580 includes a delivery protrusion 3582 configured to deliver an aerosol flow A_{DEL} towards one or more nostrils, as described herein.

The plenum portion 3570 includes a first side wall 3572 that defines an inner surface 3573. The plenum portion 3570 is configured to receive an outlet aerosol flow A_{OUT} from an aerosol preparation assembly, for example the aerosol preparation assembly 100 as described herein, via the supply line 3530. The face piece 3570 includes an end portion 3562 that includes a connection portion 3563 configured to be coupled to a supply line 3530 to receive an aerosol flow A_{OUT}. In some embodiments, an inner surface of the supply line 3530 and a side wall 3565 of the first end portion 3562 and/or the inner surface 3573 of the side wall 3572 defining a flow path FP, are configured to form a substantially continuous surface, as described above with reference to the assembly 2560. The plenum portion 3570 also includes an end side wall 3574 that defines an inner surface 3575. Furthermore, as shown in FIGS. 4B and 4C, an inner surface of a sidewall of the plenum portion 3570, for example the inner surface 3573 of the first side wall 3572 and/or the inner surface 3575 of the second side wall 3574, can define a portion of the flow path FP having a non-circular cross-sectional shape. The arrangement of the non-circular cross-section can be operative to suppress vortex flow in and through the nasal delivery protrusion 3582. Reducing flow vortices (i.e., a rotational motion of the flow about a flow axis) can reduce rainout and sputtering by reducing impaction that can result from centrifugal forces generated in vortex flows.

In some embodiments, the non-circular cross-sectional shape can have a length L along a first axis A_{V} (e.g., a vertical axis) of the cross-sectional shape and a width W along a second axis A_{H} (e.g., a horizontal axis) of the cross-sectional shape (FIG. 4B-C) such that the length L is greater than the width W. Said another way, the non-circular cross-sectional shape can have an aspect ratio. The aspect ratio can be within any suitable range, such as, for example, about 0.95, 0.90, 0.85, 0.80, 0.75, 0.70, 0.65, 0.60, or about 0.5, inclusive of all ranges therebetween. Although the second axis A_{H} is shown in FIG. 4B as being normal to the first axis A_{V}, in other embodiments, the axis A_{H} and the axis A_{V} need not be normal. In some embodiments, the non-circular cross-sectional shape can be an ellipse, an oval, or an oblong shape.

Although the cross-sectional shape in FIGS. 4B and 4C is shown as being substantially constant (e.g., a rectangular shape), in some embodiments, the flow path FP defined by the plenum portion 3570 can have a cross-sectional shape that varies along the flow path FP (i.e., that changes along the direction of flow). For example, in some embodiments, a first portion of the flow path FP defined by the first side wall 3572 of the plenum portion 3570 can have non-circular cross-sectional shape and a second portion of the flow path FP defined by a side wall 3563 included in the end portion 3562 can have a circular cross-sectional shape. In this manner, the end portion 3562 can be coupled to a standard, circular supply tube in a manner that preserves a continuous inner surface, while transitioning to a non-circular cross-sectional area to limit undesirable flow vortices.

The delivery protrusion 3582 includes an inner surface 3584 that defines a first flow path 3586. The delivery protrusion 3582 is configured to deliver an aerosol flow A_{DEL} to a nostril (e.g., the nostril of a patient) via the outlet 3588. As shown in FIG. 4C, the center line C_{L} of the nasal flow path 3586 can be curved. For example, as shown in FIG. 4C, the center line C_{L} and the first axis A_{V} can define an angle α of less than about 30 degrees, for example less than about 25 degrees, less than about 20 degrees, less than about 15 degrees, less than about 10 degrees, or less than about 5 degrees. Because the center line C_{L} is curved, the angle α can be defined between the first axis A_{V} and a line tangent to the center line C_{L} at a distance of about halfway between the intersection of the first axis A_{V} and the center line C_{L} and the exit of the nasal flow path. The angle α can be defined in at least one plane, for example a plane defined by the first axis A_{V} and the second axis A_{H}, or any other plane. Such a curved flow path can, for example enable ergonomic interface of the delivery protrusion with the nostrils of a patient, and/or alleviate rainout and/or sputtering.

In some embodiments, the delivery protrusion 3582 can be a first delivery protrusion configured to deliver a portion of the aerosol flow to a first nostril. In such embodiments, the nasal interface portion 3580 can include a second delivery protrusion (not shown) configured to convey a second portion of the aerosol flow to a second nostril such that the portion of the flow path FP that defines the non-circular cross section, for example the non-circular flow path defined by the internal surface 3573 of the first side wall 3572, is disposed between the first delivery protrusion 3582 and the second delivery protrusion. In some embodiments, the inner surface 3575 of the end side wall 3574 of the plenum portion 3570 can be a curved surface. The curved surface can be configured to redirect the second portion of the aerosol flow towards the second delivery protrusion such that end side wall 3574 is configured to fluidically isolate the flow path FP from a volume downstream from the second delivery protrusion. In some embodiments, the flow path FP can be characterized by a first cross-sectional flow area upstream from the first nasal delivery protrusion 3582 and a second cross-sectional flow area between the first delivery protrusion and the second delivery protrusion such that the second cross-sectional flow area is less than the first cross-sectional flow area.

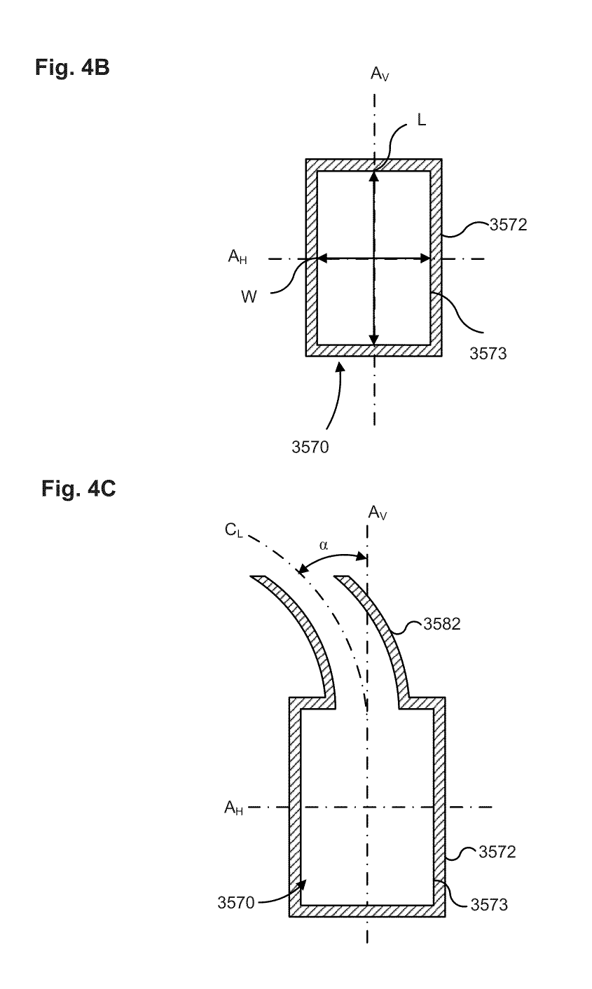

In some embodiments, a face piece can define a flow path, which can include a flow restriction. Referring now to FIG. 5, a face piece 4560 includes a plenum portion 4570 and a nasal interface portion 4580. The face piece 4560 can be included in any of the nasal cannula assemblies shown and described herein, for example the nasal cannula assembly 500, and can be used to accomplish any of the methods described herein. The nasal interface portion 4580 includes a first delivery protrusion 4582a and a second delivery protrusion 4582b. The first delivery protrusion 4582a is configured to deliver a first portion of the aerosol flow A_{DEL1} towards a first nostril and the second delivery protrusion 4582b is configured to convey a second portion of the aerosol flow A_{DEL2} towards a second nostril, as described herein.

The face piece 4560 includes an end portion 4562 that can be coupled to a supply line. The plenum portion 4570 includes a side wall 4572 that includes an inner surface 4573 defining a flow path FP configured to receive an aerosol flow, for example from a supply line. The plenum portion 4570 further includes an end side wall 4574 that includes a second surface 4575. In some embodiments, the second surface 4575 can be curved. The flow path defines a first cross-sectional flow area CS₁ upstream from the first delivery protrusion 4582a which can be defined by the inner surface 4573 of the first side wall 4572. Furthermore, the flow path FP defines a second cross-sectional flow area CS₂ such that the second cross-sectional flow area CS₂ is less than the first cross-sectional flow area CS₁. The second cross-sectional flow area can thereby produce a flow restriction in the aerosol flow path FP, for example to balance the aerosol flow. Said another way, the first portion of the delivered air flow A_{DEL1} can have a substantially similar flow rate to the second portion of the delivered air flow A_{DEL2}. This can, for example reduce rainout and/or sputtering, maintain particle size of the delivered aerosol, deliver the same mass of the aerosolized medicament to a first nostril and a second nostril of a user, and/or otherwise ensure user comfort. In some embodiments, the second cross-sectional flow area CS₂ can be defined at least in part by a second surface 4575, for example, a curved surface, of the end side wall 4574 such that the second surface 4575 is configured to redirect the second portion of the air flow towards the second delivery protrusion 4582b and the end side wall 4574 is configured to fluidically isolate the flow path FP from a volume downstream from the second delivery protrusion. In some embodiments, the end side wall 4574 can be configured to limit recirculation of the second portion of the aerosol flow at a location downstream of the second delivery protrusion 4582b, as described herein. In some embodiments, the first side wall 4572 and/or the end side wall 4574 of the plenum portion 4570 can define a portion of the flow path FP which has a non-circular cross-section, for example, rectangular, elliptical, oval, or oblong shaped, as shown above with respect to FIGS. 4A-4C.

The first delivery protrusion 4582a includes an inner surface 4584a that defines a first flow path 4586a. Similarly, the second delivery protrusion 4582b also includes an inner surface 4584b that defines a second flow path 4586b. The first delivery protrusion 4582a is configured to deliver the first portion of the aerosol flow A_{DEL1} towards the first nostril via the outlet 4588a, and the second delivery protrusion 4582b is configured to convey a second portion of the aerosol flow A_{DEL2} towards a second nostril via the outlet 4588b. In some embodiments, a center line C_{L1} of the first nasal flow path 4586a of the first delivery protrusion 4582a and a center line C_{L2} of the second nasal flow path 4586b of the second delivery protrusion 4582b can be substantially straight. In other embodiments, the center line C_{L1} and the center line C_{L2} can be curved, as shown with respect to FIG. 4C. In some embodiments, the second surface 4575 of the end side wall and the inner surface 4584b of the second delivery protrusion 4582b can form a continuous boundary between the flow path FP of the plenum portion 4570 and the second nasal flow path 4586b.

Having described above various general principles, several additional embodiments of these concepts are now described. These embodiments are only examples, and many other configurations of a nasal cannula assemblies for transnasal delivery of medicaments, are contemplated.

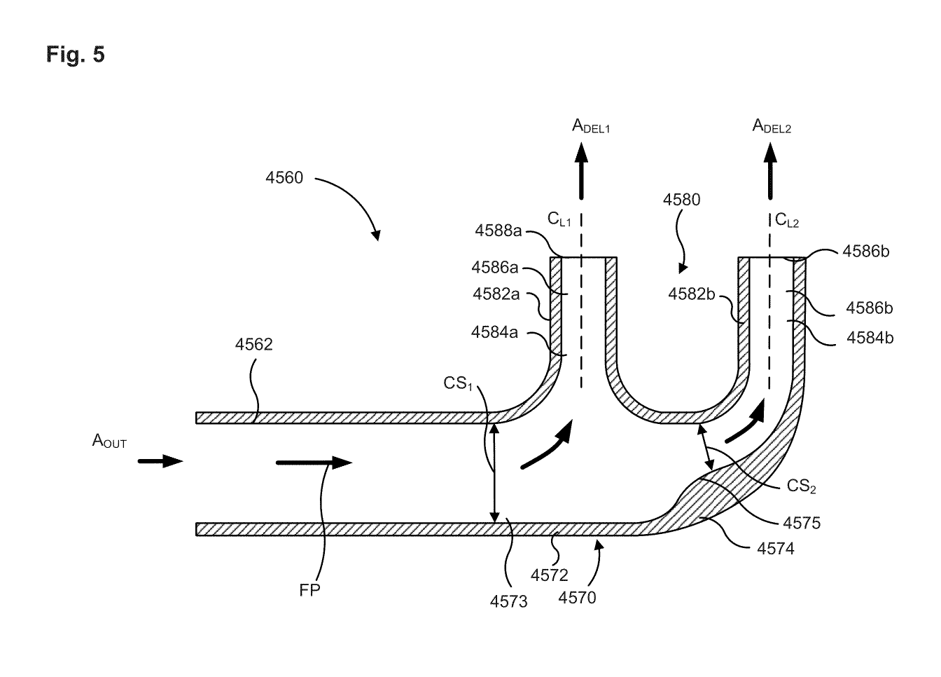

Referring now to FIG. 6-14, a face piece 5560 includes a plenum portion 5570 and a nasal interface portion 5580. The face piece 5560 can be included in any of the nasal cannula assemblies shown and described herein, for example the nasal cannula assembly 500, and can be used to accomplish any of the methods described herein. The nasal interface portion 5580 includes a first delivery protrusion 5582a and a second delivery protrusion 5582b. The first delivery protrusion 5582a is configured to deliver a first portion of the aerosol flow A_{DEL1} towards a first nostril and the second delivery protrusion 5582b is configured to convey a second portion of the aerosol flow A_{DEL2} towards a second nostril, as described herein.

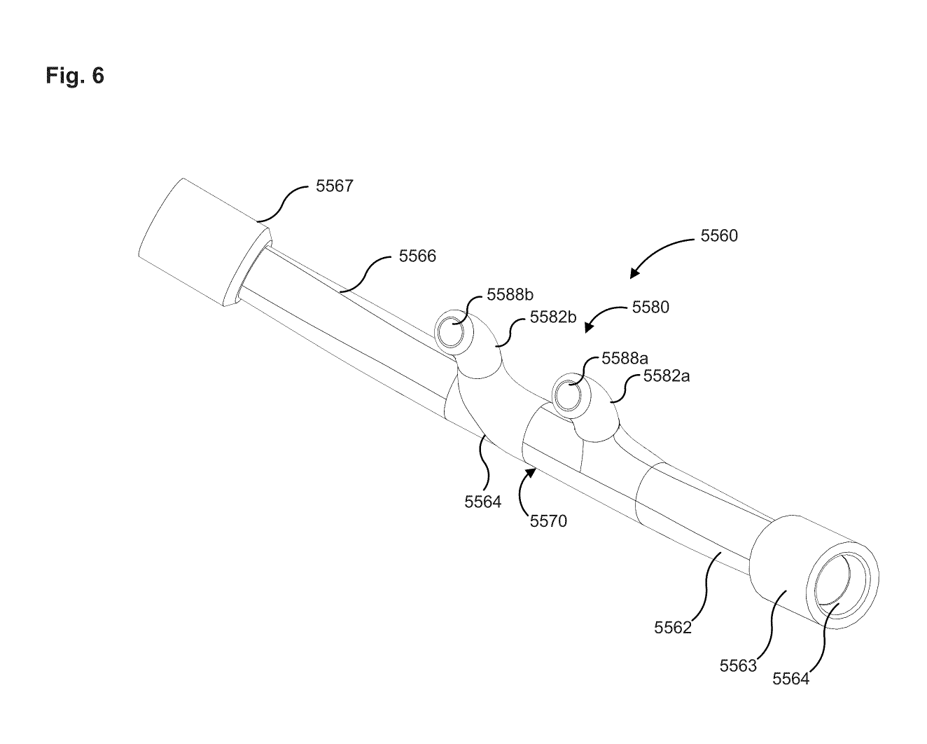

As shown in FIG. 7, the face piece includes a first end portion 5562 and a second end portion 5566. The first end portion 5562 includes connection features configured to be coupled to a supply line (not shown in FIGS. 6-14) such that the connection portion receives an aerosol flow via the supply line. In particular, the first end portion 5562 includes a connection portion 5563 that defines an opening 5564. The connection portion 5563 is configured to be removably coupled to a supply line, for example the supply line 2530 or any other supply line described herein, that delivers an outlet aerosol A_{OUT} from an aerosol preparation assembly (e.g., the aerosol preparation assembly 100) to the face piece 5560. An inner surface of the first end portion 5562 is substantially smooth and continuous with a flow path FP defined by the plenum portion 5570 and is configured to receive the outlet aerosol A_{OUT} from the supply line. The connection portion 5563 has an inner diameter (or cross-sectional size) d₁ that is substantially similar to an outer diameter (or cross-sectional size) of the supply line. This allows the supply line to be maintained in fluidic coupling with the connection portion 5563 via a friction or interference fit. Furthermore, the first end portion 5562 has an inner diameter (or cross-sectional size) d₂ downstream of the connection portion 5563 that is substantially similar to an inner diameter (or cross-sectional size) of the supply line. This arrangement ensures that when the connection portion 5563 is coupled to the supply line, an inner surface of the supply line (not shown), the inner surface of the first end portion 5562 and an inner surface 5573 of a first side wall 5572 of the plenum portion 5570, which defines the flow path FP form a substantially continuous and smooth surface. Said another way, the coupling between the supply line and the first end portion 5562 can be substantially free of bends, sudden changes in area, impediments, or other flow obstacles that can disrupt the aerosol flow or otherwise cause rainout and/or sputtering. An inner surface of the first end portion 5562 defines a transition portion 5565 where the internal diameter (or cross-sectional size) d₂ of the first end portion transitions to the internal diameter (or cross-sectional size) d₄ of the plenum portion 5570. The transition is substantially smooth and continuous, i.e. free of bends, impediments or flow obstacles. In some embodiments, the inner diameter, size or otherwise cross-section of the connection portion d₁ can be about 6 mm, and the inner diameter d₂ of the first end portion 5562 downstream of the connection portion 5563 can be about 4.6 mm.

As shown, the opening 5564 defined by the connection portion 5563 is flared so that the supply line can be easily aligned with and/or inserted into the connection portion 5563. In some embodiments, the connection portion 5563 can included threads, luer lock threads, grooves, notches, indents, detents, a snap-fit mechanism, or any other suitable mechanism for removably coupling the supply line. In some embodiments, the supply line can be fixedly coupled to the connection portion 5563, for example, via adhesives, fusion bonding, heat welding, or the likes.

The second end portion 5566 includes mounting features for receiving a mounting member, for example a solid tube or a dummy tube (not shown, e.g., a face piece tube that does not convey any portion of the aerosol flow). The second end portion 5566 is operative to mount the face piece in proximity to the nostrils of a patient cooperatively with the supply line and/or a face piece tube. The second end portion 5566 includes a mounting portion 5567 which defines an opening 5568 configured to be coupled to the mounting member. The second end portion 5566 is fluidically isolated from the plenum portion 5570 by an end side wall 5574 of the plenum portion. The mounting 5567 defines an internal diameter (or cross-sectional size) d₃ which can be substantially similar to the internal diameter (or cross-sectional size) d₁ of the connection portion 5563. The second end portion 5566 also defines an internal diameter (or cross-sectional size) d₄ which can be substantially similar to the internal diameter (or cross-sectional size) d₂ of the first end portion 5562, such that the second end portion 5566 is configured to matingly receive the mounting member (not shown in FIGS. 6-14). The opening 5568 defined by the mounting portion 5567 is flared so that the mounting member can be easily aligned and/or coupled to the mounting member via a simple friction fit mechanism. In some embodiments, the mounting portion 5567 can included threads, luer lock threads, grooves, notches, indents, detents, a snap-fit mechanism, or any other suitable mechanism for removably coupling the mounting member. In some embodiments, the mounting member can be fixedly coupled to the mounting portion 5567, for example, via adhesives, fusion bonding, heat welding, or the likes.

Each of the first end portion 5562 and the second portion 5566 define an angle γ (e.g., a “bend angle”) with respect to a longitudinal axis A_{L} of the flow path defined by the plenum portion 5570, which is shown as being substantially horizontal. For example the angle γ can be less than about 30 degrees, less than about 25 degrees, or less than about 20 degrees, inclusive of all ranges therebetween such that the face piece can ergonomically conform to the natural curvature of a face of a user.

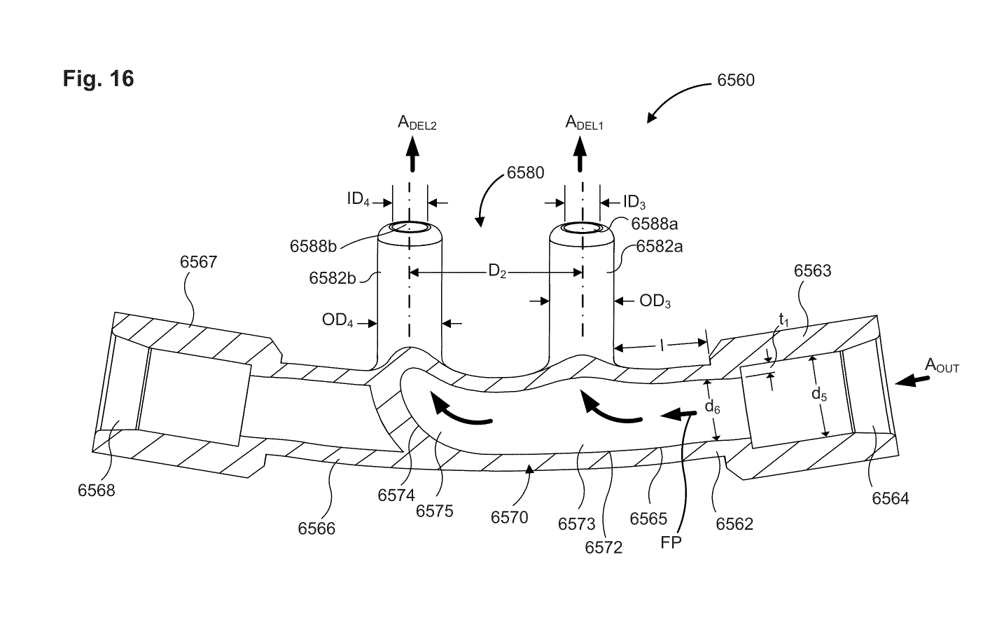

In some embodiments, the face piece 5560 can be dimensioned to be suitable for use by an adult patient. In such embodiments, the face piece 5560 can be dimensioned to have a length measured from an end of the connection portion 5563 to the mounting portion 5567 along the longitudinal axis A_{L} of about 76 mm. Moreover, the first delivery protrusion 5582a and the second delivery protrusion 5582b can have a center to center spacing D₁ of about 17 mm (e.g., substantially similar to the spacing between the nostrils of an adult user) as shown in FIG. 17.

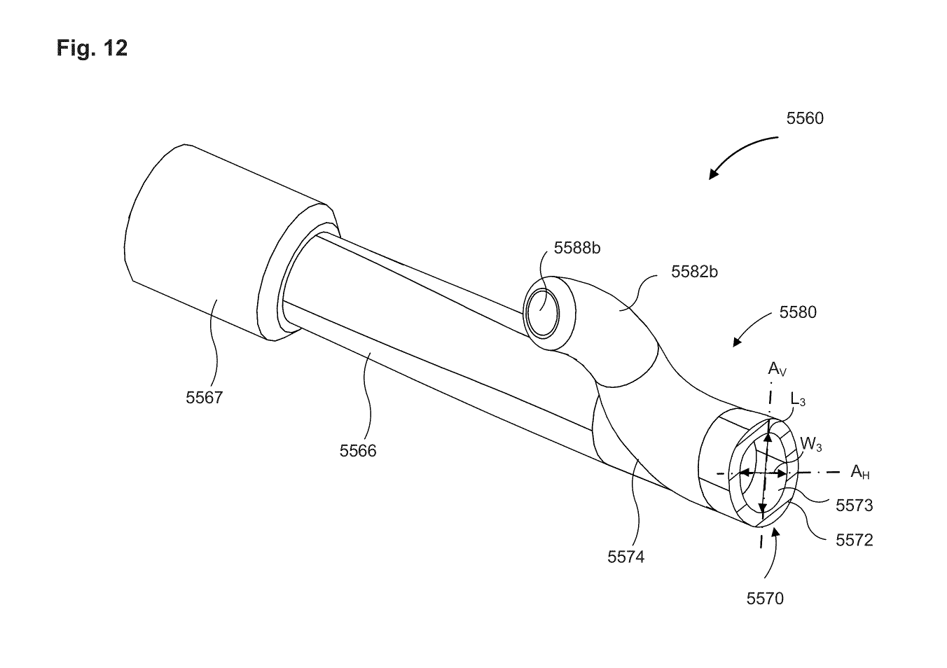

As described before, the first side wall 5572 of the plenum portion 5570 has the inner surface 5573 that defines the flow path FP. The plenum portion 5570 further includes an end side wall 5574 that has a curved surface 5575. The curved surface 5575 is configured to redirect a portion of the aerosol towards the second delivery protrusion 5582b, as shown by the arrow BB (FIG. 7). Furthermore, the end side wall 5572 defines an angle of curvature θ configured to limit the recirculation, and/or otherwise doubling back of the second portion of the aerosol flow. Recirculation can occur if the aerosol flow experiences a change in flow direction of greater than about 90 degrees such that at least a portion of the aerosol flow is recirculated back in to the flow path FP of the plenum portion 5570. Recirculation can cause impaction and vortex generation which can increase rainout and/or sputtering. Furthermore, recirculation can reduce the amount of the aerosol delivered to the second delivery protrusion, resulting in imbalanced aerosol flow. Thus, the end side wall 5574 can be configured to define an angle of curvature θ of less than about 90 degrees, for example less than about 85 degrees, less than about 80 degrees, less than about 75 degrees, or less than about 70 degrees. Moreover, the curved surface 5575 is configured to form a substantially continuous boundary between the flow path FP of the plenum portion 5570 and a flow path defined by an inner surface 5586b of the second delivery protrusion 5582b (FIG. 13). This can ensure that the transition of the aerosol flow is substantially smooth and continuous, which can substantially reduce rainout and/or sputtering.

The end side wall 5574 also serves to fluidically isolate the flow path FP of the plenum portion 5570 from a volume downstream of the second delivery protrusion 5582b. Similarly stated, the end side wall 5574 defines the face piece 5560 as a unilateral face piece, in that aerosol flow A_{OUT} enters the face piece from a single direction.

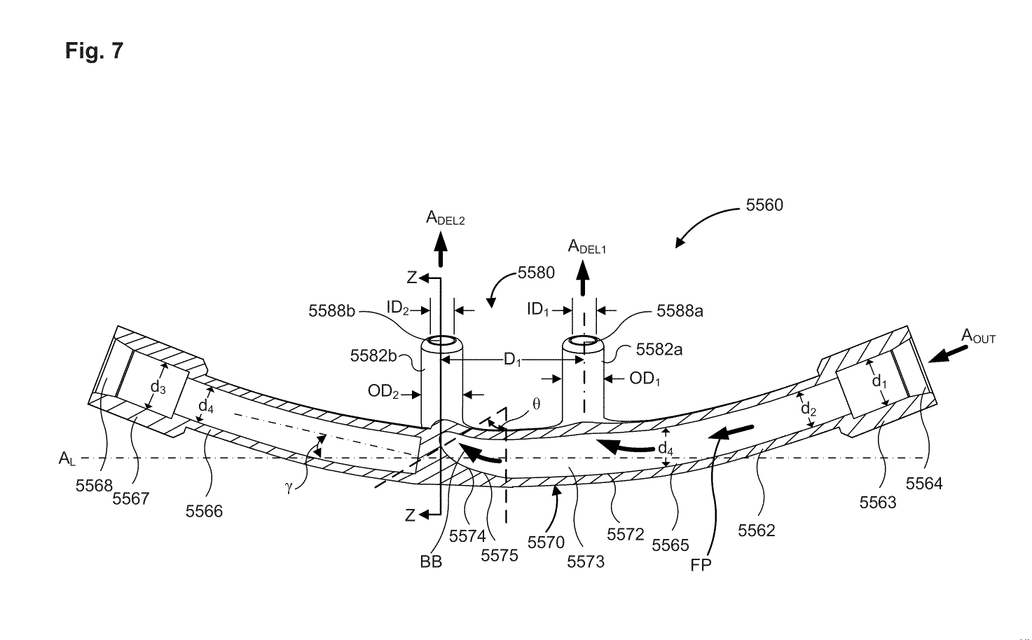

As shown in FIG. 8 the inner surface 5573 of the first side wall 5572 defines a first cross-sectional flow area CS₃ (shown in two dimensions for clarity) upstream of the first delivery protrusion 5582a. The end side wall 5574 defines a second cross-sectional flow area CS₄ (shown in two dimensions for clarity) between the first delivery protrusion 5582a and the second delivery protrusion 5582b, such that the second cross-sectional flow area CS₄ is less than the first cross-section flow area CS₃. Said another way, the end side wall 5574 defines a flow restriction within the flow path FP of the plenum portion 5570. The flow restriction can be sized and/or configured to balance the aerosol flow between the first delivery protrusion 5582a and the second delivery protrusion 5582b such that the flow rate of the first portion of the delivered aerosol A_{DEL1} and the flow rate of the second portion of the delivered aerosol A_{DEL2} are substantially equal. For example, in some embodiments, the end side wall 5574 can be configured such that a flow rate of the first portion of the aerosol flow A_{DEL1} is within about 10% of a flow rate of the second portion of the aerosol flow A_{DEL2}. Furthermore, the transition from the first cross-sectional flow area CS₃ to the second cross-sectional flow area CS₄ can be substantially smooth and continuous, such that the aerosol flow does not encounter any impediment, obstacles or otherwise sudden changes in area that can cause turbulence or impaction.

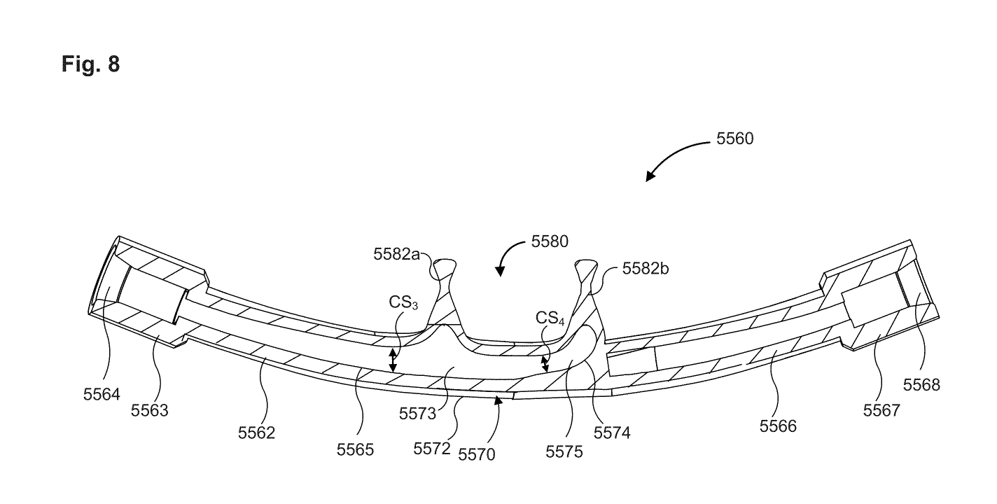

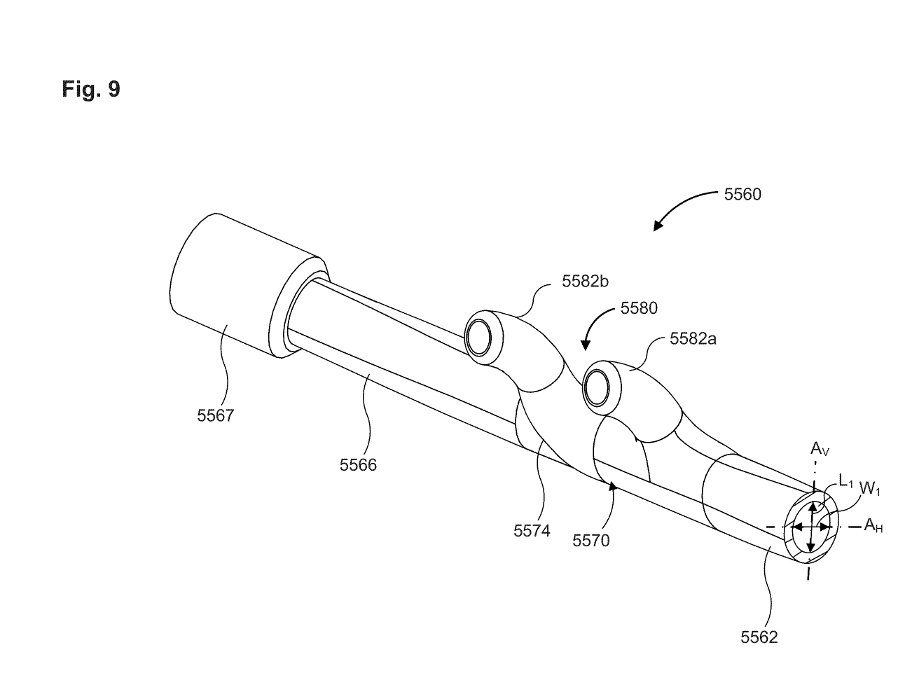

The cross-sectional flow area can be of any suitable shape, and/or can change shapes and/or size as a function of the position along the flow path. In some embodiments, the cross-sectional flow area defined by the face piece 5570 can be non-circular and/or oblong. As shown in FIGS. 9-14, the cross-sectional flow area defined by the face piece 5570 can have a length along a first axis A_{V} and a width along a second axis A_{H} that vary along the flow path. FIG. 9 shows a first side cross-section view of the first end portion 5562. The first end portion 5562 has a first length L₁ along the first axis A_{V} and a first width W₁ along the second axis A_{H}. As shown in FIG. 9, the first length L₁ and the first width W₁ are substantially equal (i.e., equal to the inner diameter, size or otherwise cross-section d₂ of the first end portion 5562) such that the first end portion has a first cross-sectional shape which is substantially circular. FIG. 10 shows a second side cross-section of the face piece 5560 taken along the plenum portion 5570 at a location downstream of the first end portion 5562 and upstream of the first delivery protrusion 5582a. The second side cross-section defines a second cross-sectional shape that has a second length L₂ along the first axis A_{V} and a second width W₂ along the second axis A_{H}. The second length L₂ is substantially greater than the second width W₂ and the second cross-sectional shape is substantially elliptical and/or oblong (i.e., non-circular). Said another way, the cross-sectional shape defined by the flow path of the face piece 5560 varies from a circular cross-sectional shape at a first location upstream from the first delivery protrusion 5582a to a second, non-circular cross-sectional shape (i.e., the elliptical or oblong shape) at a second location downstream from the first location (but still upstream from the first delivery protrusion 5582a). The transition is substantially smooth and continuous such that the aerosol flow does not encounter any impaction or experiences sudden changes in flow direction that can cause sedimentation and increase rainout and/or sputtering. In some embodiments, the second length L₂ can be substantially similar to the first length L₁. In some embodiments, the second length L₂ can be substantially larger than the first length L₁. In some embodiments, the length L₂ and/or the length L₁ can be about 5.60 mm, and the width W₂ and/or the width W₁ can be about 4.20 mm. In some embodiments, the aspect ratio of the second width W₂ to the second length L₂ can be about 0.95, 0.9, 0.85, 0.8, 0.75, or 0.7.

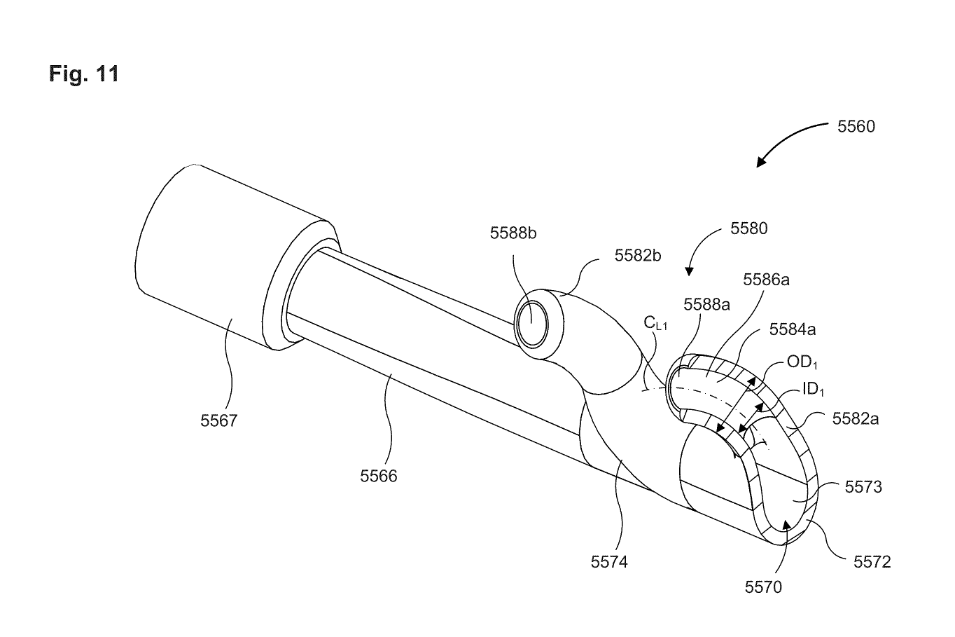

FIG. 12 shows a side-cross section of the plenum portion 5570 taken at a location in between the first delivery protrusion 5582a and the second delivery protrusion 5582b which defines a third cross-sectional shape. The third cross-sectional shape defines a third length L₃ measured along the first axis A_{V} and a third width W₃ measured along the second axis A_{H}. The third length L₃ is substantially greater than the third width W₃ such that the third cross-sectional shape is non-circular (e.g., is oblong and/or elliptical). The third length L₃ can be substantially similar in size to second length L₂ and the third width W₃ can be substantially similar in size to the second width W₂. The non-circular and/or elliptical shape defined by the plenum portion 5570 can reduce and/or eliminate the generation of vortices in the aerosol flow therethrough. This, in turn, can substantially reduce inertial and or impaction sedimentation of the aerosol droplets, thereby reducing rainout and/or sputtering. In some embodiments, the length L₂ and/or the length L₃ can be about 5.60 mm, and the width W₂ and/or the width W₃ can be about 4.20 mm. In some embodiments, the aspect ratio of the third width W₃ to the third length L₃ can be about 0.95, 0.9, 0.85, 0.8, 0.75, or 0.7.

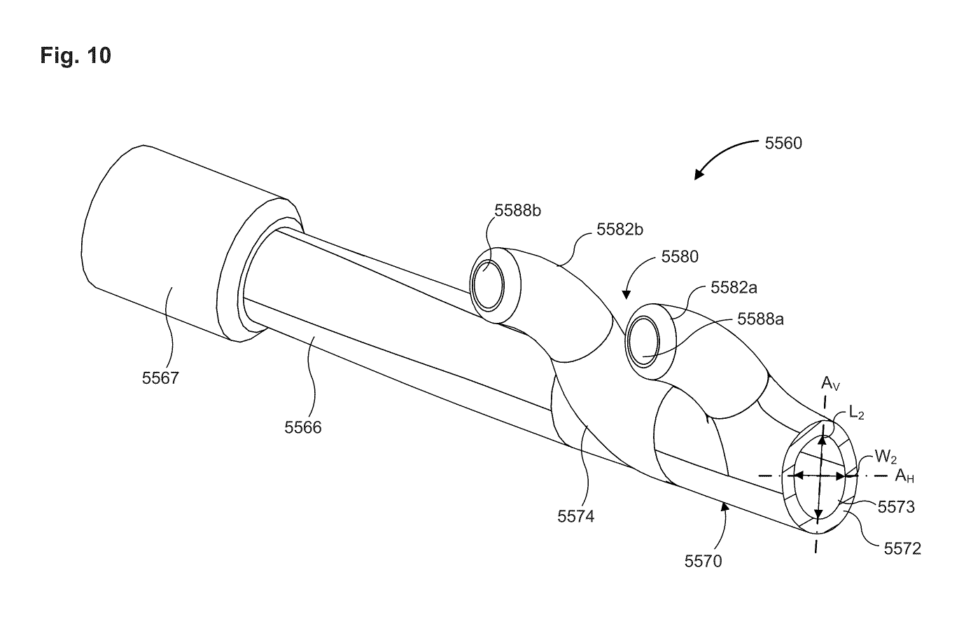

As described herein, the nasal interface portion 5580 includes a first delivery protrusion 5582a and a second delivery protrusion 5582b. The first delivery protrusion 5582a has an inner surface 5584a that defines a first nasal flow path 5586a configured to deliver the first portion of the delivered aerosol A_{DEL1} to the first nostril via a first outlet 5588a (FIG. 11). The first delivery protrusion 5582a has a circular cross-section and defines an inner diameter ID₁ and an outer diameter OD₁, which are substantially constant over the length of the first delivery protrusion 5582a. Similarly, the second delivery protrusion 5582b has an inner surface 5584b that defines a second nasal flow path 5586b configured to deliver the second portion of the delivered aerosol A_{DEL2} to the second nostril via a second outlet 5588b (FIG. 13). The second delivery protrusion 5582b also has a circular cross-section and defines an internal diameter ID₂ and an outer diameter OD₂, which are also substantially constant over the length of the second delivery protrusion 5582b. In some embodiments, the inner diameter ID₁ of the first delivery protrusion 5582a and the inner diameter ID₂ of the second delivery protrusion 5582b can be substantially equal, for example about 2.5 mm or about 3.0 mm.

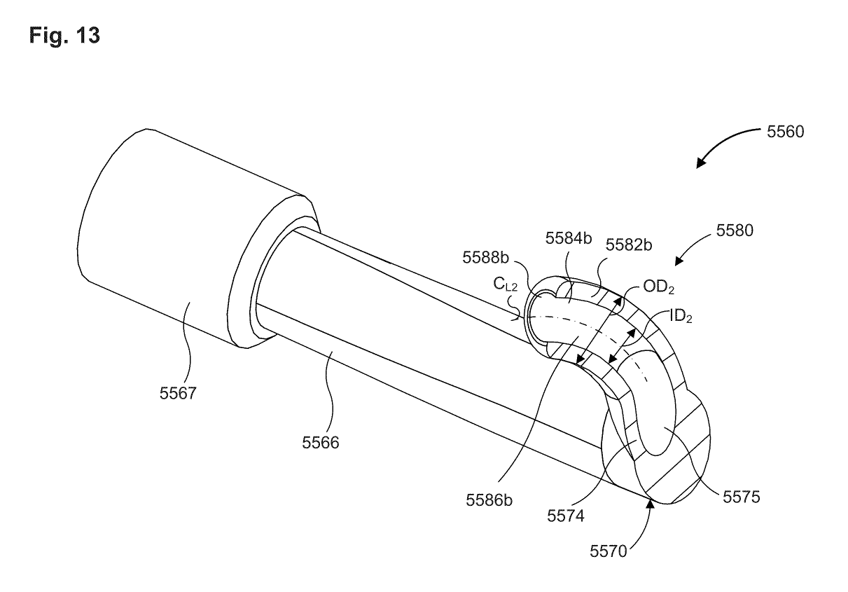

As described herein, and shown in the side cross-section view of FIG. 13, the curved surface 5575 defined by the end side wall 5574 of the plenum portion 5570 is substantially continuous with the inner surface 5584b of the second delivery protrusion 5582b. Thus, the curved surface 5575 forms a substantially smooth and/or continuous boundary between the flow path FP of the plenum portion 5570 and the second nasal flow path 5586b. Furthermore, the center line C_{L1} of the first flow path 5586a of the first delivery protrusion 5582a and the center line C_{L2} of the second flow path 5586b of the second delivery protrusion 5582b can be curved with respect to the first axis A_{V} of the cross-sectional flow area of the face piece 5560. As shown in the side cross-section view of FIG. 14 taken along the line ZZ shown in FIG. 7, the center line C_{L2} and the first axis A_{V}, and/or the center line C_{L1}, and the first axis A_{V} can define an angle β which can be less than about 30 degrees (e.g., less than about 25 degrees, less than about 20 degrees, or less than about 15 degrees). Because the center line C_{L1} is curved, the angle β can be defined between the first axis A_{V} and a line tangent to the center line C_{L1} at a distance of about halfway between the intersection of the first axis A_{V} and the center line C_{L1} and the exit of the nasal flow path. The curvature of the first delivery protrusion 5582a and the second delivery protrusion 5582b can, for example, reduce rainout and/or sputter, and/or be configured to conform to a curvature of the face of the patient to enhance patient comfort.

In some embodiments, the face piece 5560 or any other face piece described herein, can be configured to receive an aerosol flow, for example from the aerosol preparation assembly 100, which includes an aerosol of liquid particles having a VMD from about 0.5 μm to about 2.5 μm. The face piece 5560 can be configured, such that an amount of liquid particles deposited within the face piece 5560 is less than about ten percent of an amount of the liquid particles conveyed from the first delivery protrusion 5582a and the second delivery protrusion 5582b after thirty minutes of operation. For example, the amount of liquid particles deposited can be less than about 5%, less than about 2%, less than about 1%, less than about 0.5%, less than about 0.4%, less than about 0.3% less than 0.2%, or less than about 0.1%. In some embodiments, the aerosol of liquid particles can have a flow rate of less than about 200 μl/min and a gas flow rate in the range of about 1 L/min to about 3 L/min. In some embodiments, the face piece 5560 can be configured such that no sputter is emitted from the first delivery protrusion 5582a and the second delivery protrusion 5582b after thirty minutes.

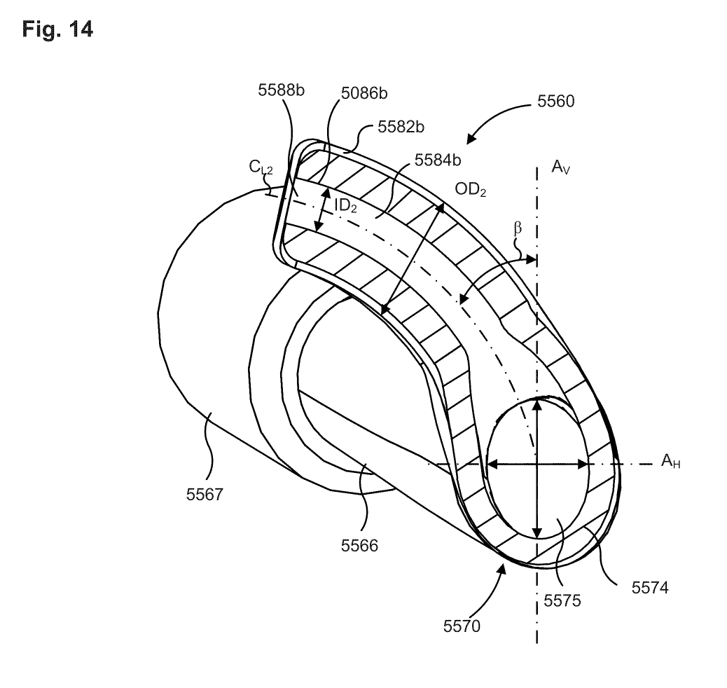

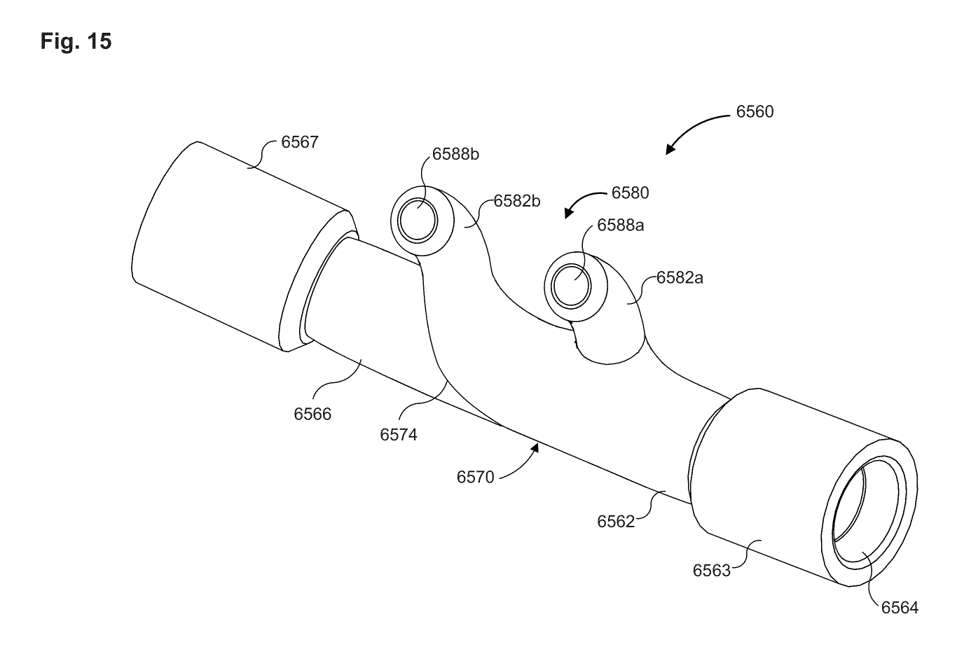

Although described above as being suitable for use in an adult patient, in other embodiments, any of the face pieces and/or cannula assemblies described herein can be configured for use on any patient having any anatomical considerations. In some embodiments, for example, a face piece can be configured for use by a pediatric patient, i.e., a patient between the ages of about 0 years (including pre-term babies) to about 5 years old. For example, FIG. 15 and FIG. 16 show a pediatric face piece 6560 which includes a plenum portion 6570 and a nasal interface portion 6580. The pediatric face piece 6560 can be included in any of the nasal cannula assemblies shown and described herein, for example the nasal cannula assembly 500, and can be used to accomplish any of the methods described herein. The nasal interface portion 6580 includes a first delivery protrusion 6582a and a second delivery protrusion 6582b. The first delivery protrusion 6582a is configured to deliver a first portion of the aerosol flow A_{DEL1} towards a first nostril and the second delivery protrusion 6582b is configured to convey a second portion of the aerosol flow A_{DEL2} towards a second nostril, as described herein. The pediatric face piece 6560 is substantially similar to the adult face piece 5560, but is sized and shaped to fit a pediatric patient.

The pediatric face piece 6560 includes a first end portion 6562 and a second end portion 6566. The first end portion 6562 includes connection features configured to be coupled to a supply line such that the connection portion receives an aerosol flow. The first end portion 6562 includes a connection portion 6563 that defines an opening 6564. The connection portion 6563 is configured to be removably coupled to a supply line, for example the supply line 2530 or any other supply line described herein, that delivers an outlet aerosol A_{OUT} to the face piece 6560 from an aerosol preparation assembly, for example the aerosol preparation assembly 100. An inner surface of the first end portion 6562 is substantially continuous with an inner surface 6573 of a first side wall 6572 of the plenum portion 6570, such that inner surface 6573 defines a flow path FP configured to receive the outlet aerosol A_{OUT} from the supply line. The connection portion 6563 has an inner diameter d₅ that is substantially equal to an outer diameter of the supply line. Moreover, the first end portion 6562 has an inner diameter d₆ downstream of the connection portion 6563 that is substantially equal to an inner diameter of the supply line. This ensures that an inner surface of the supply line and an inner surface of the first end portion form a substantially smooth and continuous surface free of bends, impediments, flow obstacles or the like, which can cause vortices or eddies that can increase rainout and/or sputtering. An inner surface of the first end portion 6562 defines a transition portion 6565 where the internal diameter d₆ of the first end portion transitions in to an internal diameter of the plenum portion 6570. The transition is smooth and continuous such that the first end portion 6562 and the flow path FP defined by the plenum portion 6570 are substantially continuous. In some embodiments, the inner diameter of the connection portion d₅ can be about 6 mm, and the inner diameter d₆ of the first end portion 6562 downstream of the connection portion 6563 can be about 4.6 mm. The opening 6564 defined by the connection portion 6563 is flared so that the supply line can be easily coupled with the connection portion 6563 via a simple friction fit mechanism. In some embodiments, the connection portion 6563 can included threads, luer lock threads, grooves, notches, indents, detents, a snap-fit mechanism, or any other suitable mechanism for removably coupling the supply line. In some embodiments, the supply line can be fixedly coupled to the connection portion 6563, for example, via adhesives, fusion bonding, heat welding, or the likes.

The second end portion 6566 includes mounting features for receiving a mounting member, for example a solid tube or a dummy tube, configured to mount the face piece in proximity to the nostrils of a patient cooperatively with the supply line and/or a face piece tube. The second end portion 6566 includes a mounting portion 6567 which defines an opening 6568 configured to be coupled to the mounting member. The second end portion 6566 is fluidically isolated from the plenum portion 6570 by a side wall 6574 of the plenum portion 6570. The second end portion 6566 can be substantially similar to the second end portion 5566 described with respect to the adult face piece 5560 and is therefore not described in further detail herein. The first end portion 6562, the second end portion 6566 and the plenum portion 6570 can be configured such that the pediatric face piece 6560 has a length of about 34 mm measured from an end of the first end portion 6562 to an end of the second end portion 6566.

As described before, the first side wall 6572 of the plenum portion 6570 has an inner surface 6573 that defines the flow path FP configured to receive the aerosol flow A_{OUT} from the supply line (not shown in FIGS. 15 and 16). A first portion of the outlet aerosol A_{OUT} is conveyed from the plenum portion 6570 to the first delivery protrusion 6582a. The plenum portion 6570 further includes an end side wall 6574 that has a curved surface 6575 configured to redirect a portion of the aerosol towards the second delivery protrusion 6582b. The plenum portion 6570 of the pediatric face piece 6560 can be substantially similar to the plenum portion 5570 of the adult face piece 5560 and is therefore, not described herein in further detail.

The first delivery protrusion 6582a has an inner surface 6584a that defines a first nasal flow path 6586a configured to deliver the first portion of the delivered aerosol A_{DEL1} to the first nostril via a first outlet 6588a. The first delivery protrusion 6582a has a circular cross-section and defines an inner diameter ID₃ and an outer diameter OD₃. Similarly, the second delivery protrusion 6582b has an inner surface 6584b that defines the second nasal flow path 6586b configured to deliver the second portion of the delivered aerosol A_{DEL2} to the second nostril via a second outlet 6588b (FIG. 16). The second delivery protrusion 6582b also has a circular cross-section and defines an internal diameter ID₄ and an outer diameter OD₄. In some embodiments, the inner diameter ID₃ of the first delivery protrusion 6582a and the inner diameter ID₄ of the second delivery protrusion 6582b can be substantially equal, for example about 2.5 mm or about 3 mm, which can be spaced apart by a distance D₂, for example of about 12 mm (e.g., substantially equal to the spacing between the first nostril and the second nostril of a pediatric patient). The first delivery protrusion 6582a and the second delivery protrusion 6582b can be substantially similar in structure and functioning to the first delivery protrusion 5582a and the second delivery protrusion 5582b, shown and described with respect to the adult face piece 5560, and are therefore, not described in further detail herein.

In some embodiments, the face piece 6560 or any other face piece described herein, can be configured to receive an aerosol flow, for example from the aerosol preparation assembly 100, which includes an aerosol of liquid particles having a VMD from about 0.5 μm to about 2.5 μm, for example about 1 μm, about 1.5 μm, or about 2 μm. The nasal cannula assembly, for example the nasal cannula assembly 500, can be configured to convey a first portion of the aerosol flow to a first nostril via the first delivery protrusion 6582a and a second portion of the aerosol flow to a second nostril via the second delivery protrusion 6582b, such that an amount of liquid particles deposited within the face piece 6560 is less than about ten percent of an amount of the liquid particles conveyed from the first delivery protrusion 6582a and the second delivery protrusion 6582b after thirty minutes of operation. For example, the amount of liquid deposited can be less than about 5%, less than about 2%, less than about 1%, less than about 0.5%, less than about 0.4%, less than about 0.3% less than 0.2%, or less than about 0.1% after thirty minutes of operation. In some embodiments, the aerosol of liquid particles can have a flow rate of less than about 200 μl/min and a gas flow rate in the range of about 1 L/min to about 3 L/min. In some embodiments, the face piece 6560 can be configured such that no sputter is emitted from the first delivery protrusion 6582a and the second delivery protrusion 6582b after thirty minutes of operation.

The adult face piece 5560 and the pediatric face piece 6560 described herein each include a single plenum portion (5570 and 6570, respectively) configured to direct flow of the aerosol to each of the first delivery protrusion 5582a and 6582a, respectively, and the second delivery protrusion 5582b and 6582b, respectively. In other embodiments, a plenum portion of a face piece can include bifurcation or structure therein such that the aerosol flow can be divided within the plenum portion into a first aerosol flow and second aerosol flow. For example, FIG. 17 shows a face piece 7560 including a plenum portion 7570 and a nasal interface portion 7580. The face piece 7560 can be included in any of the nasal cannula assemblies shown and described herein, for example the nasal cannula assembly 500, and can be used to accomplish any of the methods described herein. The nasal interface portion 7580 includes a first delivery protrusion 7582a and a second delivery protrusion 7582b. The first delivery protrusion 7582a is configured to deliver a first portion of the aerosol flow A_{DEL1} towards a first nostril and the second delivery protrusion 7582b is configured to convey a second portion of the aerosol flow A_{DEL2} towards a second nostril, as described herein. An end portion 7562 of the face piece 7560 includes connection features and is configured to be coupled to a supply line, for example the supply line 2530 to receive an outlet aerosol A_{OUT} from an aerosol preparation assembly, for example the aerosol preparation assembly 100. As shown, the face piece 7560 includes a single inlet to receive the outlet aerosol A_{OUT}, and is thus another example of a unilateral flow face piece.

The plenum portion 7570 includes a first side wall 7572 having an inner surface 7573 that defines a flow path FP configured to receive the aerosol flow A_{OUT} from the supply line. A first portion of the outlet aerosol A_{OUT} is conveyed from the plenum portion to the first delivery protrusion 7582a. A second portion of the outlet aerosol A_{OUT} is conveyed from the plenum portion to the second delivery protrusion 7582b. In particular, the plenum portion 7570 includes a bifurcation side wall 7576 which divides the flow path FP of the plenum portion 7570 in to a first flow path and a second flow path. The plenum portion 7570 includes a first end side wall 7574a having a first curved surface 7575a configured to redirect a first portion of the aerosol flow towards the first delivery protrusion 7582a as shown by the arrow CC. The first curved surface 7575a is configured to form a continuous boundary between the flow path FP of the plenum portion 7570 and a first flow path defined by an inner surface 7586a of the first delivery protrusion 7582a. The plenum portion 7570 also includes a second end side wall 7574b having a second curved surface 7575b configured to redirect a second portion of the aerosol flow towards the second delivery protrusion 7582b, as shown by the arrow DD. The second curved surface 7575b is configured to form a continuous boundary between the flow path FP of the plenum portion 7570 and a second flow path defined by an inner surface 7586b of the second delivery protrusion 7582b. Thus, in use, the aerosol flow first divides into the first portion and the second portion before undergoing the change in direction from the plenum portion 7570 towards the first delivery protrusion 7582a and the second delivery protrusion 7582b. Thus, the transition of the aerosol flow form the flow path FP of the plenum portion 5570 to the first nasal flow path and the second nasal flow path is substantially smooth and continuous, for example, free of sharp bends, impediments, obstacles, or the like, thereby reducing rainout and/or sputtering.

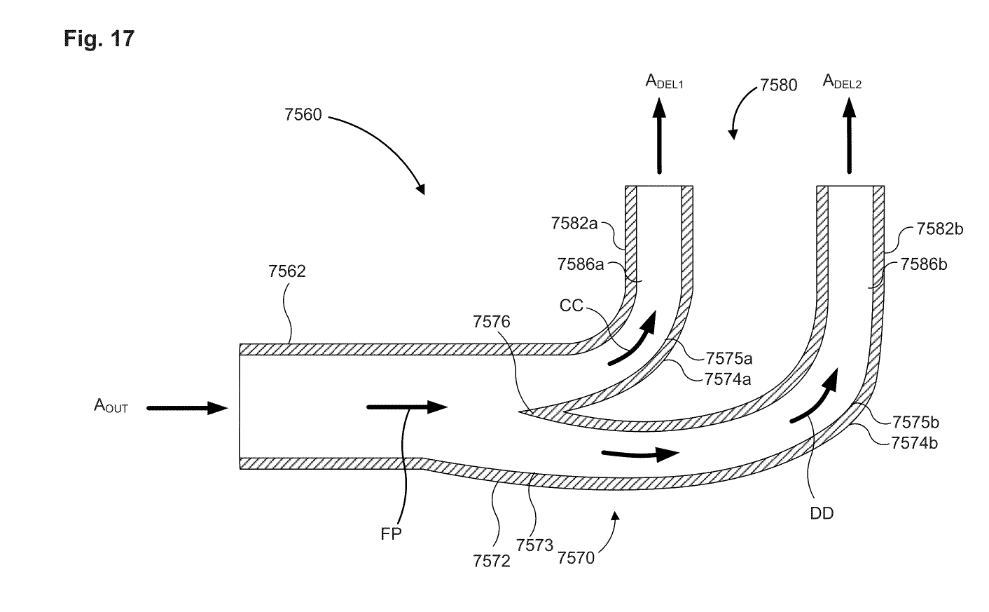

Although shown as including a bifurcation structure before any substantial change in direction towards the delivery protrusions, in other embodiments, a plenum portion of a face piece can include a late bifurcation such that the aerosol flow is divided in to a first aerosol flow and a second aerosol flow within a nasal interface portion. For example, referring now to FIG. 18, a face piece 8560 includes a plenum portion 8570 and a nasal interface portion 8580. The face piece 8560 can be included in any of the nasal cannula assemblies shown and described herein, for example the nasal cannula assembly 500, and can be used to accomplish any of the methods described herein. The nasal interface portion 8580 includes a first delivery protrusion 8582a and a second delivery protrusion 8582b. The first delivery protrusion 8582a is configured to deliver a first portion of the aerosol flow A_{DEL1} towards a first nostril and the second delivery protrusion 8582b is configured to convey a second portion of the aerosol flow A_{DEL2} towards a second nostril. An end portion 8562 of the face piece 8560 includes connection features and is configured to be coupled to a supply line, for example the supply line 530, to receive an outlet aerosol A_{OUT} from an aerosol preparation assembly, for example the aerosol preparation assembly 100.

The plenum portion 8570 includes a first side wall 8572 defining an inner surface 8573 and an end side wall 8574 which defines a curved surface 8575. The plenum portion 8570 includes a bifurcation side wall 8576 disposed in between the first delivery protrusion 8582a and the second delivery protrusion 8582b. The bifurcation side wall 8576 is disposed such that a linear distance H₁ measured from an edge of a first outlet 8588a of the first delivery protrusion 8582a (or measured from a second outlet 8688b of the second delivery protrusion 8582b) to an outer surface of the bifurcation side wall 8576 is less than a distance H₂ measured from the edge of the first outlet 8588a to an outer surface of the first side wall 8572 of the plenum portion 8570. Said another way, the aerosol flow path FP defined by the plenum portion 8570 divides into a first nasal flow path 8586a defined by an inner surface 8584a of the first delivery protrusion 8582a, and a second nasal flow path 8586b defined by the inner surface 8584b of the second delivery protrusion 8582b late in the aerosol flow. Thus, in such embodiments, the flow path of the aerosol flow changes direction towards the first delivery protrusion 8582a and the second delivery protrusion 8582b before dividing into a first portion and a second portion. Furthermore, the curved surface 8575 defined by the end side wall 8574 is substantially smooth and continuous with a second inner surface 8578 defined by a second side wall 8577 of the plenum portion 8570. The second inner surface 8578 can be substantially continuous with the inner surface 8584b of the second delivery protrusion. Thus, the second portion of the aerosol flow can be redirected towards the second delivery protrusion 8582b by the curved surface 8575 such that the redirected portion of the aerosol flow travels substantially within the plenum portion 8570 before entering the second nasal flow path 8586b of the second delivery protrusion 8582b. This can, for example reduce impaction and/or sedimentation to reduce rainout and/or sputtering.

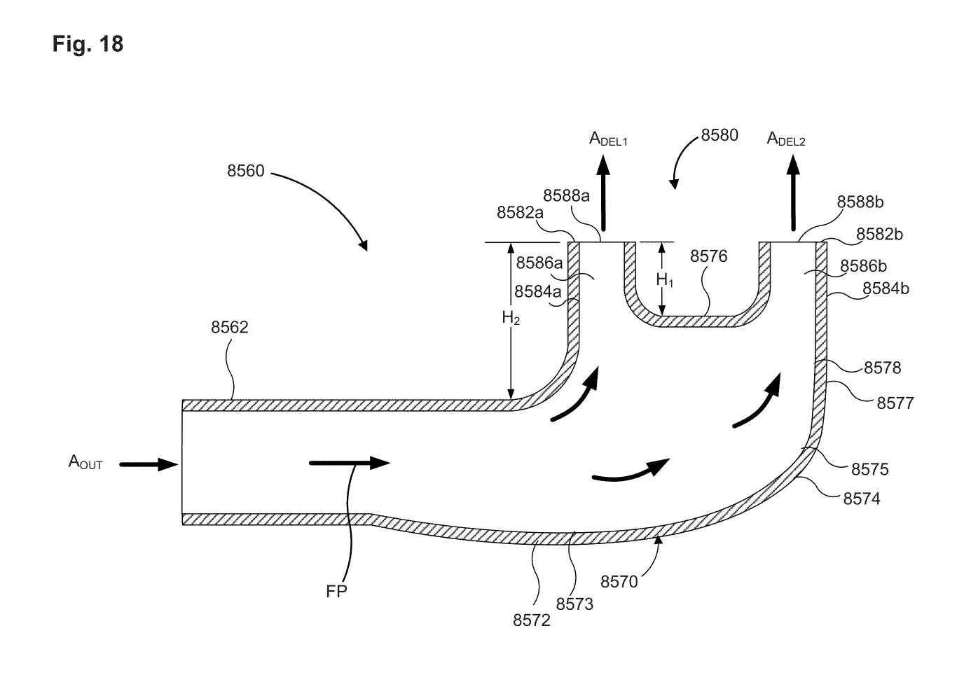

In some embodiments, a face piece can be configured such that a longitudinal axis defined by a plenum portion of the face piece is substantially parallel to nasal flow paths of a first and a second delivery protrusion defined by the face piece. In this manner, a supply line can be substantially parallel to the delivery protrusions, thereby limiting the number of bends, which can reduce rainout and/or sputtering. For example, FIG. 19 shows a face piece 9560 including a plenum portion 9570 and a nasal interface portion 9580. The face piece 9560 can be included in any of the nasal cannula assemblies shown and described herein, for example the nasal cannula assembly 500, and can be used to accomplish any of the methods described herein. The nasal interface portion 9580 includes a first delivery protrusion 9582a and a second delivery protrusion 9582b. The first delivery protrusion 9582a is configured to deliver a first portion of the aerosol flow A_{DEL1} towards a first nostril and the second delivery protrusion 9582b is configured to convey a second portion of the aerosol flow A_{DEL2} towards a second nostril. An end portion 9562 of the face piece 9560 includes connection features and is configured to be coupled to a supply line, for example the supply line 530 configured to receive an outlet aerosol A_{OUT} from an aerosol preparation assembly, for example the aerosol preparation assembly 100.

The plenum portion 9570 includes a first side wall 9574a that defines a first curved surface 9575a configured to redirect a portion of the aerosol flow towards the first nostril. The plenum portion 9570 also includes a second side wall 9574b that defines a second curved surface 9575b configured to redirect a portion of the aerosol flow towards the second nostril. The plenum portion 9570 is disposed such that a longitudinal axis A_{L1} of the plenum portion 9570 is substantially parallel to a center line C_{L1} of a first nasal flow path 9586a defined by an inner surface 9584a of the first delivery protrusion 9582a. The longitudinal axis A_{L1} is also parallel to a center line C_{L2} of a second nasal flow path 9586b defined by an inner surface 9584b of the second delivery protrusion 9582b. As shown in FIG. 19, the first side wall 9574a and the second side wall 9574b are configured to redirect and/or recirculate the first portion and the second portion of the aerosol flow, respectively in to the plenum portion 9570 and ultimately towards the first delivery protrusion 9582a and the second delivery protrusion 9582b, respectively. For example, the curved surface 9575a of the first side wall 9574a and/or the curved surface 9575b of the second side wall 9574b, can define an angle of curvature θ₁ of greater than about 90 degrees, for example about 120, about 150, about 180 degrees, about 210 degrees, or even higher inclusive of all ranges therebetween. The nasal interface portion 9580 can be substantially similar to the nasal interface portion 5580 shown and described with respect to the face piece 5560, or any other nasal interface portion included in any face piece described herein.

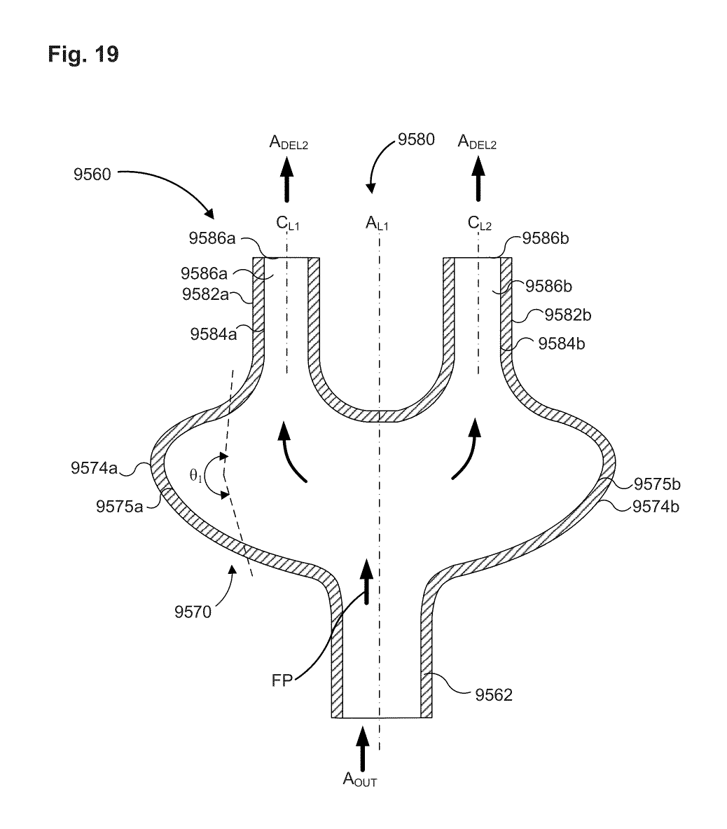

In some embodiments, a longitudinal axis defined by the flow path of a plenum portion included in a face piece can be substantially parallel to the nasal flow paths of the delivery protrusions and the plenum portion can further be configured to limit recirculation. For example, referring now to FIG. 20, a face piece 10560 includes a plenum portion 10570 and a nasal interface portion 10580. The face piece 10560 can be included in any of the nasal cannula assemblies shown and described herein, for example the nasal cannula assembly 500, and can be used to accomplish any of the methods described herein. The plenum portion 10570 defines a longitudinal axis A_{L2} substantially parallel to a center line C_{L3} of a first nasal protrusion 10582a and a center line C_{L1} of a second nasal protrusion 10582a. The plenum portion 10570 includes a first side wall 10574a defining a first curved surface 10575a, and a second side wall 10574b defining a second curved surface 10575b. The first side wall 10574a and the second side wall 10574b are configured to reduce and/or eliminate recirculation of the first portion and the second portion of the aerosol flow at a location downstream of the first delivery protrusion 10582a and the second delivery protrusion 10582b, respectively. For example, the curved surface 10575a of the first side wall 10574a and/or the curved surface 10575b of the second side wall 10574b, can define an angle of curvature θ₂ of less than about 90 degrees, for example about 85 degrees, about 80 degrees, about 75 degrees, about 70 degrees, or even lower, inclusive of all ranges therebetween. The nasal interface portion 10580 can be substantially similar to the nasal interface portion 5580 shown and described with respect to the face piece 5560, or any other nasal interface portion included in any face piece described herein.

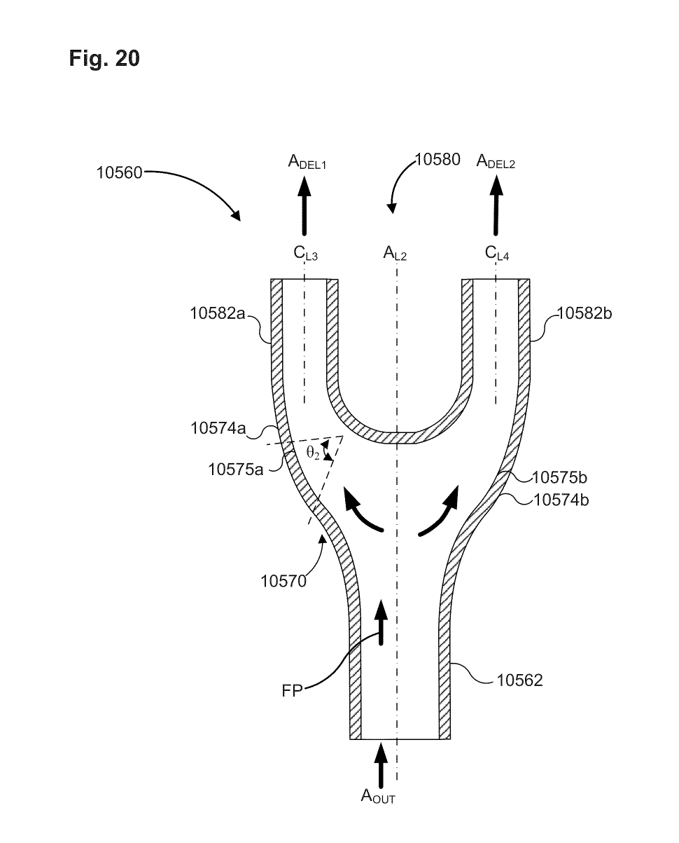

In some embodiments, a longitudinal axis defined by the flow path of a plenum portion included in a face piece can be substantially parallel to the nasal flow paths of the delivery protrusions and the plenum portion can include a bifurcation side wall. For example, referring now to FIG. 21, a face piece 11560 includes a plenum portion 11570 and a nasal interface portion 11580. The face piece 11560 can be included in any of the nasal cannula assemblies shown and described herein, for example the nasal cannula assembly 500, and can be used to accomplish any of the methods described herein. The plenum portion 11570 defines a longitudinal axis A_{L3} substantially parallel to a center line C_{L5} of a first nasal protrusion 11582a and a center line C_{L6} of a second nasal protrusion 11582a. The plenum portion 11570 includes a first side wall 11574a defining a first curved surface 11575a, and a second side wall 11574b defining a second curved surface 11575b. The plenum portion 11570 includes a bifurcation side wall 11576 configured to divide the flow path FP of the plenum portion 11570 into a first flow path shown by the line EE and a second flow path shown by the FF such that the aerosol flow is divided into a first aerosol flow and a second aerosol flow substantially within the plenum portion 11570. Similarly stated, the bifurcation side wall 11576 includes a protrusion configured to redirect the incoming aerosol flow A_{OUT} while minimizing the recirculation and/or production of eddies in the same. The nasal interface portion 11580 can be substantially similar to the nasal interface portion 5580 shown and described with respect to the face piece 5560, or any other nasal interface portion included in any face piece described herein.

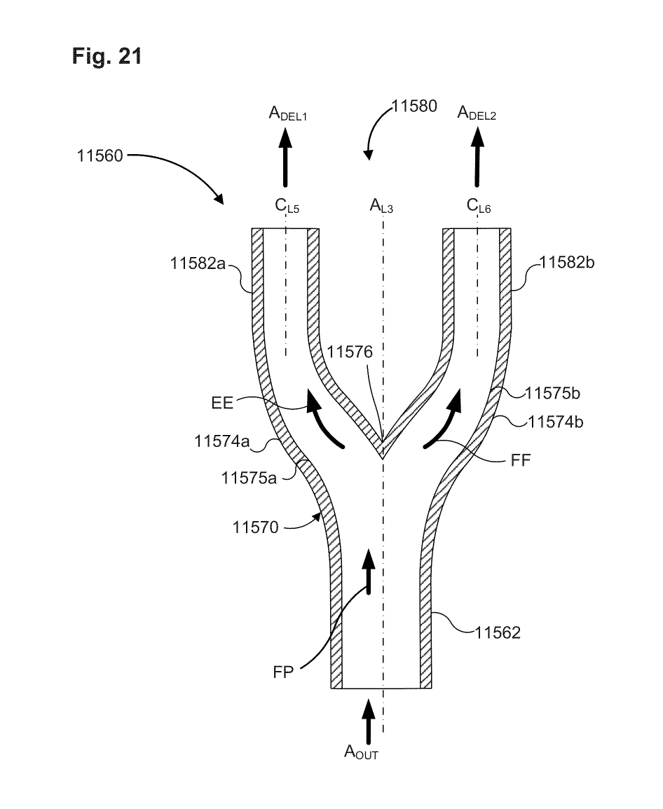

In some embodiments a nasal interface portion, for example a first delivery protrusion and a second delivery protrusion included in the nasal interface portion, of a face piece can be configured to modify the flow characteristics of the aerosol flow (e.g., to reduce rainout and/or sputtering and/or to enhance patient comfort). In some embodiments, delivery protrusions included in a face piece can be configured to suppress, reduce and/or eliminate swirling in the flow. By reducing the rotational motion of the aerosol flow, impaction (and the associated rainout and sputtering) can be reduced. Referring now to FIG. 22A-B, a face piece 12560 includes a plenum portion 12570 and a nasal interface portion 12580. The face piece 12560 can be included in any of the nasal cannula assemblies shown and described herein, for example the nasal cannula assembly 500, and can be used to accomplish any of the methods described herein. The face piece 12560 includes a connection portion 12562 configured to be coupleable to a supply line, for example the supply line 530 or any other supply line described herein, to receive an outlet aerosol A_{OUT} from an aerosol preparation assembly, for example aerosol preparation assembly 100. The plenum portion 12570 includes a first side wall 12572 having an inner surface 12573 that defines a flow path FP of the plenum portion 12570. The plenum portion 12570 also includes an end side wall 12574 having a curved surface 12575. The plenum portion 12570 can be substantially similar to the plenum portion 1570, 3570, 4570, or any other plenum portions described herein with respect to any embodiments of the face piece described herein.

The nasal interface portion 12580 includes a first delivery protrusion 12582a and a second delivery protrusion 12582b. The first delivery protrusion 12582a is configured to deliver a first portion of the aerosol flow A_{DEL1} towards a first nostril and the second delivery protrusion 12582b is configured to convey a second portion of the aerosol flow A_{DEL2} towards a second nostril. Each of the first delivery protrusion 12582a and the second delivery protrusion 12582b (collectively referred to as “delivery protrusions 12582”) has a cross-sectional length L along a first axis A₁ of a cross-sectional shape defined by the delivery protrusions, and a cross-sectional width W along a second axis A₂ of the cross-sectional shape. The length L and the width W can be substantially different, such that the delivery protrusions 12582a, 12582b have a non-circular cross-section. For example, the width W can be longer than the length L such that the cross-sectional shape defined by the delivery protrusions 12582 is oblong (e.g., elliptical or oval). Such a shape can suppress swirling (e.g., vortices or cyclones) in the aerosol flow entering and/or flowing within the delivery protrusions 12582. Swirling can centrifuge the aerosol droplets on the walls of the delivery protrusions 12582 which can increase rainout/and or swirling. Thus, the swirl suppressing delivery protrusions 12582 can reduce rainout and/or sputtering.

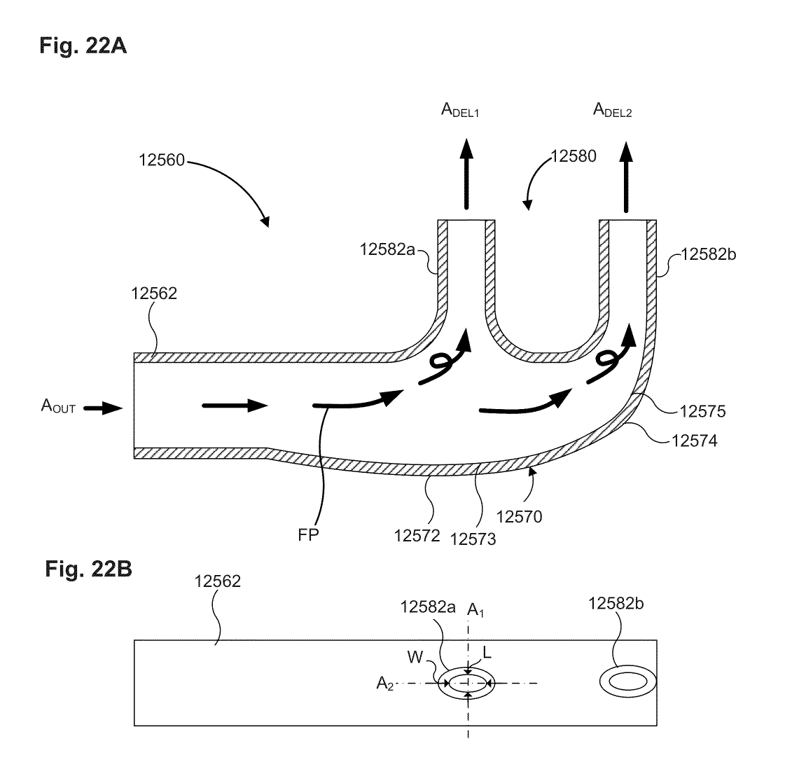

In some embodiments, delivery protrusions included in a face piece can be shaped and sized to provide a tight fit with the nostrils of a user. This arrangement can minimize and/or reduce leakage of medicaments and improve deposition efficiency. For example as shown in FIG. 23, a face piece 13560 includes a plenum portion 13570 and a nasal interface portion 13580. The face piece 13560 can be included in any of the nasal cannula assemblies shown and described herein, for example the nasal cannula assembly 500, and can be used to accomplish any of the methods described herein. The face piece 13560 includes a connection portion 13562 configured to be coupleable to a supply line, for example the supply line 530 or any other supply line described herein, to receive an outlet aerosol A_{OUT} from an aerosol preparation assembly, for example aerosol preparation assembly 100. The plenum portion 13570 includes a first side wall 13572 having an inner surface 13573 that defines a flow path of the plenum portion 13570. The plenum portion 13570 also includes an end side wall 13574 having a curved surface 13575. As shown in FIG. 23, the curved surface 13575 of the end side wall 13574 defines an angle of curvature φ greater than about 90 degrees, for example, 100 degrees, 120 degrees, 150 degrees, 180 degrees, or 210 degrees, or even higher inclusive of all ranges therebetween, such that the end side wall 13574 recirculates flow back into the plenum portion 13570. This arrangement produces a reservoir or “bulge” at the second end portion of the face piece 13560 that can function to collect any rainout produced during use, thus reducing the likelihood that the collected rainout will be conveyed towards the exit of the delivery protrusions 12582a, 12582b (resulting in undesirable sputter). In some embodiments, however, the curved surface 13575 of the end side wall 13574 can define a radius of curvature φ less than about 90 degrees to limit recirculation, as described before herein.

The nasal interface portion 13580 includes a first delivery protrusion 13582a and a second delivery protrusion 13582b such that the first delivery protrusion 13582a is configured to deliver a first portion of the aerosol flow A_{DEL1} towards a first nostril and the second delivery protrusion 13582b is configured to convey a second portion of the aerosol flow A_{DEL2} towards a second nostril. Each of the first delivery protrusion 13582a and the second delivery protrusion 13582b (collectively referred to as “delivery protrusions 13582”) can have an outer diameter, size or otherwise cross-section OD substantially similar to an internal diameter of the nostrils of a patient. The delivery protrusions 13582 can thereby fit snugly within the nostrils of the patient such that substantially all of the aerosol flow to the nostrils of the patient is via the face piece 13570.

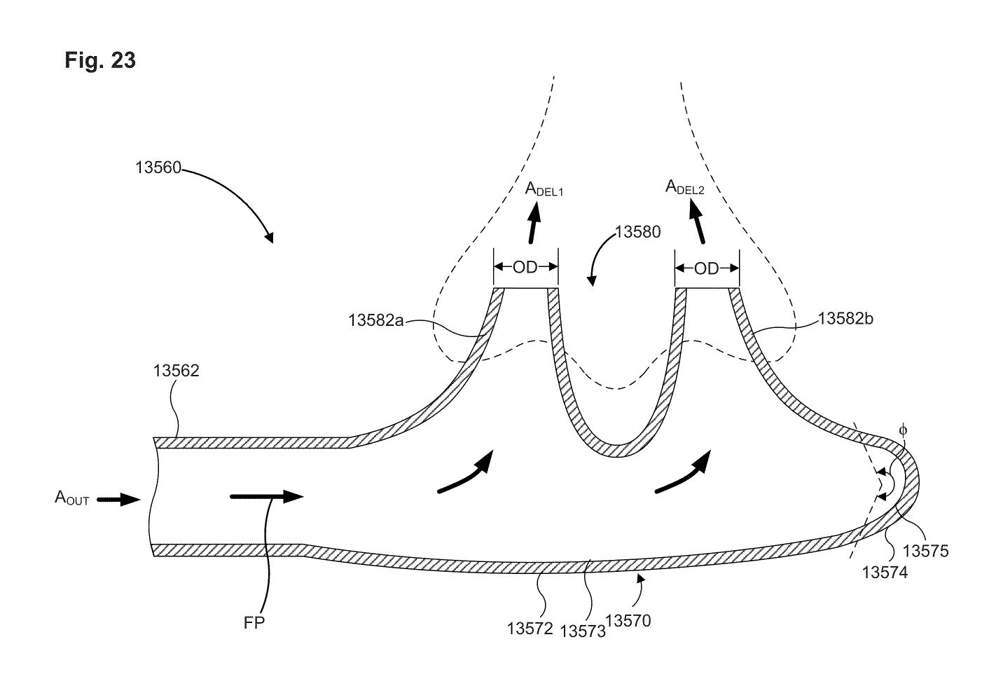

In some embodiments, delivery protrusions included in a face piece can be flared (e.g., tapered in an outwardly extending manner). For example, as shown in FIG. 24, a face piece 14560 includes a plenum portion 14570 and a nasal interface portion 14580. The face piece 14560 can be included in any of the nasal cannula assemblies shown and described herein, for example the nasal cannula assembly 500, and can be used to accomplish any of the methods described herein. The face piece 14560 includes a connection portion 14562 configured to be coupleable to a supply line (not shown in FIG. 24), for example the supply line 530 or any other supply line described herein, to receive an outlet aerosol A_{OUT} from an aerosol preparation assembly, for example aerosol preparation assembly 100. The face piece 14560 includes a plenum portion 14570 and a nasal interface portion 14580. The plenum portion 14570 can be substantially similar to the plenum portion 1570, 3570, 4570, or any other plenum portions described herein with respect to any embodiments of the face piece described herein. The nasal interface portion 14580 includes a first delivery protrusion 14582a and a second delivery protrusion 14582b (collectively referred to as “delivery protrusions 14582”) such that the first delivery protrusion 14582a is configured to deliver a first portion of the aerosol flow A_{DEL1} towards a first nostril and the second delivery protrusion 14582b is configured to convey a second portion of the aerosol flow A_{DEL2} towards a second nostril. The delivery protrusions 14582 define a first inner diameter d₁ at the base of the delivery protrusions and a second inner diameter at an outlet of the delivery protrusions 14582 such that d₂ is greater than d₁ (producing the flared delivery protrusions). The flared delivery protrusions 14582 can reduce the velocity of the aerosol flow of the delivered aerosol A_{DEL1} and A_{DEL2} into the nostrils (e.g., due to the increased exit area), for example to reduce rainout and/or sputtering and/or for patient comfort.

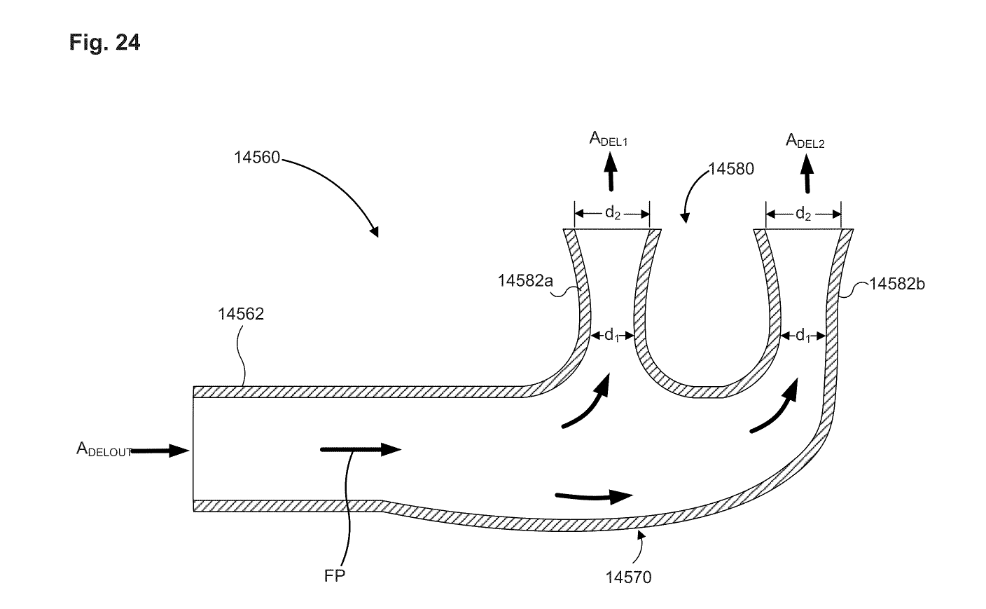

In some embodiments, the delivery protrusions of a face piece can be tapered to form a nozzle at the exit thereof. For example as shown in FIG. 25, a face piece 15560 includes a plenum portion 15570 and a nasal interface portion 15580. The face piece 15560 can be included in any of the nasal cannula assemblies shown and described herein, for example the nasal cannula assembly 500, and can be used to accomplish any of the methods described herein. The face piece 15560 includes a connection portion 15562 that can be substantially similar to the connection portion 5563 described with reference to the face piece 5560 and is therefore not described in further detail herein. The plenum portion 15570 can be substantially similar to the plenum portion 1570, 3570, 4570, or any other plenum portions described herein with respect to any embodiments of the face piece described herein. The face piece 15560 includes a nasal interface portion 15580 that includes a first delivery protrusion 15582a and a second delivery protrusion 15582b (collectively referred to as “delivery protrusions 15582”) such that the first delivery protrusion 15582a is configured to deliver a first portion of the aerosol flow A_{DEL1} towards a first nostril and the second delivery protrusion 15582b is configured to convey a second portion of the aerosol flow A_{DEL2} towards a second nostril. The delivery protrusions 15582 define a first inner diameter d₃ at the base of the delivery protrusions and a second inner diameter d₄ at an outlet of the delivery protrusions 15582 such that d₃ is greater than d₄ and the delivery protrusions are tapered such that the flow area reduces in the direction of the flow. Said another way, the delivery protrusions 15582 resemble nozzles. The tapered delivery protrusions 15582 can increase the air flow of the delivered aerosol A_{DEL1} and A_{DEL2} in to the nostrils, for example to reduce rainout and/or sputtering or to produce a desired exit velocity into the nose.

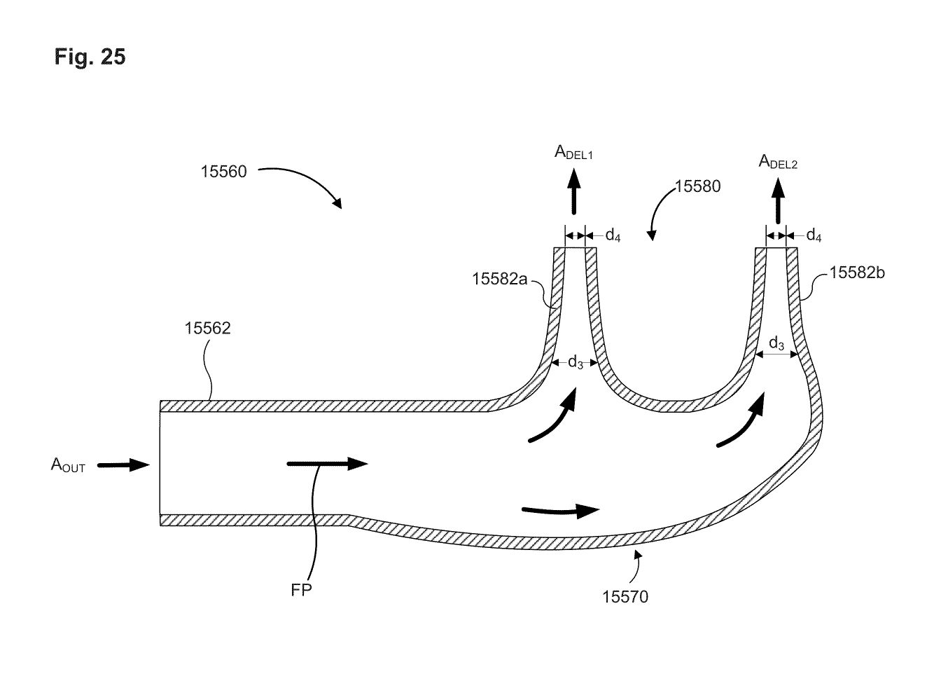

Although the face pieces shown above (e.g. the face piece 5560) are configured to deliver the aerosol flow to each nostril (i.e., they include two delivery protrusions), in other embodiments, a face piece can be configured to communicate fluid through only one delivery protrusion. Referring now to FIG. 26, a face piece 16560 includes a plenum portion 16570 and a nasal interface portion 16580. The face piece 16560 can be included in any of the nasal cannula assemblies shown and described herein, for example the nasal cannula assembly 500, and can be used to accomplish any of the methods described herein. The face piece 16560 includes a connection portion 16562 that can be substantially similar to the connection portion 5563 described with reference to the face piece 5560 and is therefore not described in further detail herein. The plenum portion 16570 can be substantially similar to the plenum portion 1570, 3570, 4570, or any other plenum portions described herein with respect to any embodiments of the face piece described herein. The nasal interface portion 16580 includes a first delivery protrusion 16582a and a second delivery protrusion 16582b. The first protrusion 16582a can be blocked (e.g., with a valve or filled with sealant) or alternatively can be a solid protrusion such that all of an outlet aerosol A_{OUT} communicated in to a flow path FP defined by the plenum portion 16570 is delivered to the second delivery protrusion 16582b. In some embodiments, the nasal interface portion 16580 can include only one delivery protrusion, for example the second delivery protrusion 16582b.

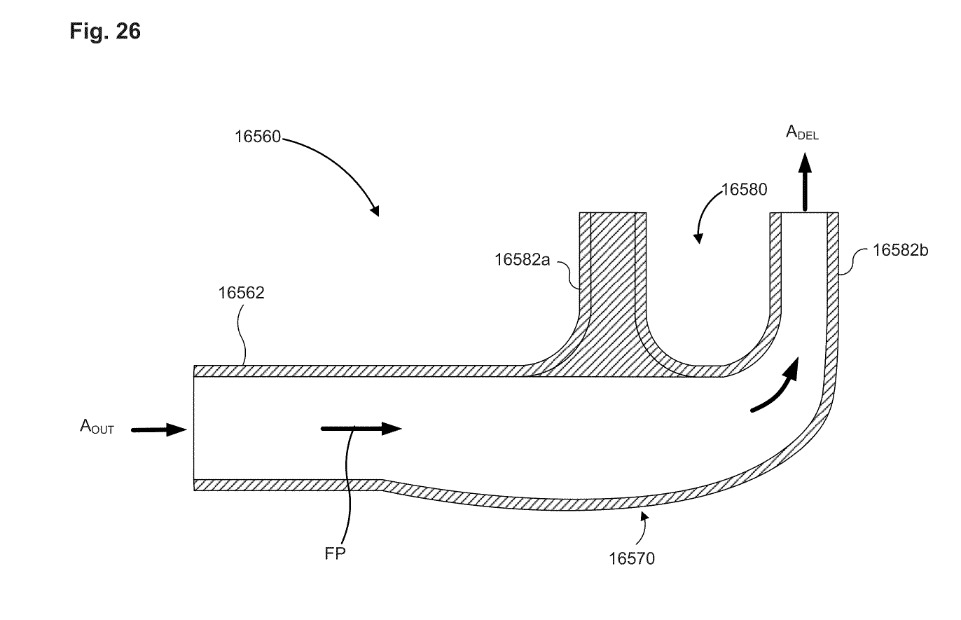

In some embodiments, a face piece can be devoid of delivery protrusions. For example, as shown in FIG. 27, a face piece 17560 includes a plenum portion 17570 and a nasal interface portion 17580. The face piece 17560 can be included in any of the nasal cannula assemblies shown and described herein, for example the nasal cannula assembly 500, and can be used to accomplish any of the methods described herein. The face piece 17560 includes a connection portion 17562 that can be substantially similar to the connection portion 5563 described with reference to the face piece 5560 and is therefore not described in further detail herein. The plenum portion 17570 can be substantially similar to the plenum portion 1570, 3570, 4570, or any other plenum portions described herein with respect to any embodiments of the face piece described herein. The nasal interface portion 17580 includes a first delivery outlet 17588a and a second delivery outlet 17588b (collectively referred to as “the delivery outlets”) configured to deliver a first portion of the aerosol flow to a first nostril and a second portion of the aerosol flow to a second nostril of a user, respectively. The delivery outlets can, for example help to reduce a velocity of the aerosol exiting the nasal interface portion 17580 such the aerosol can be breathed in naturally by the user, and is not forced into the nostrils of the user. The slower flow rate can further reduce impaction which can reduce rainout and/or sputtering. In some embodiments, the nasal interface portion 17580 can include more than two openings. For example, in some embodiments, the nasal interface portion 17580 can include a membrane that includes a multiplicity of pores.

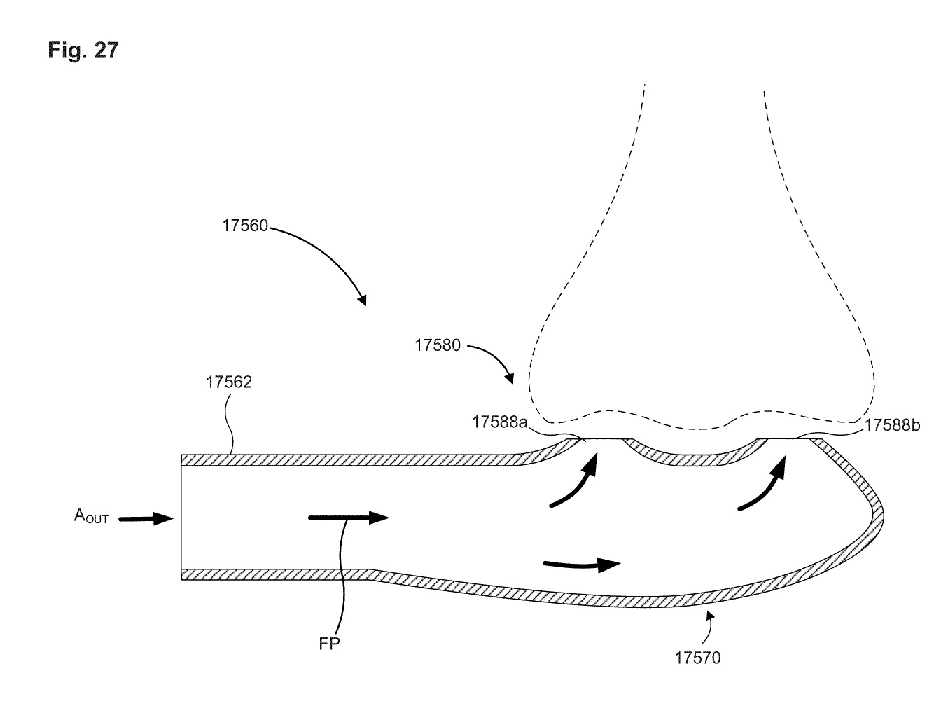

In some embodiments, a nasal interface portion can include a single outlet and/or can include a portion that is disposed about and/or outside of the nostrils. For example, as shown in FIG. 28, a face piece 18560 includes a plenum portion 18570 and a nasal interface portion 18580. The face piece 18560 can be included in any of the nasal cannula assemblies shown and described herein, for example the nasal cannula assembly 500, and can be used to accomplish any of the methods described herein. The face piece 18560 includes a connection portion 18562 that can be substantially similar to the connection portion 5563 described with reference to the face piece 5560 and is therefore not described in further detail herein. The plenum portion 18570 can be substantially similar to the plenum portion 1570, 3570, 4570, or any other plenum portions described herein with respect to any embodiments of the face piece described herein. The nasal interface portion 18580 includes a delivery outlet 18588 which can be sized and shaped to surround a nose of a user. In some embodiments, the delivery outlet 18588 can fit snugly around the nose such that substantially all of the aerosol flow is delivered to the nostrils of the user. The delivery outlet can be configured to reduce the velocity and/or flow rate of the aerosol such that the aerosol can be naturally breathed in by the user.

In some embodiments, a face piece, for example the face piece 1560, 3560, 4560, 5560, or any other face piece shown and described herein can include features, structure and/or mechanisms to minimize, reduce, and/or otherwise eliminate rainout, or to collect and/or transport aerosol droplets that can cause rainout or sputtering. For example, in some embodiments, the face piece can be formed from a material that is hydrophobic, for example Teflon, such that the aqueous aerosol droplets do not condense on an inner surface of the face piece. In some embodiments, the internal surface of the face piece can be configured to included micro- or nano-features, for example posts, pillars, voids, cavities, dimples, or any other features configured to render the internal surface hydrophobic. Such features can be introduced via physical means, for example in a molding or casting process, sand blasting, or deposition of micro or nanoparticles, or via chemical means for example, solvent or acid etching. In some embodiments, the surface chemistry of an inner surface of the face piece can be modified to make the inner surface hydrophobic, for example via an oxygen plasma treatment, ultra violet light treatment or coating with a hydrophobic self-assembled monolayer. In some embodiments, the inner surface of the face piece can be coated with a material that is hydrophobic such as, for example silica nano coating, precipitated calcium carbonate, zinc oxide polystyrene nano-composite, manganese oxide polystyrene nano-composite, oils, lipids, fats, NeverWet™, P2i™, Aculon™, Lotus Leaf coatings, or any other hydrophobic coatings or combination thereof.

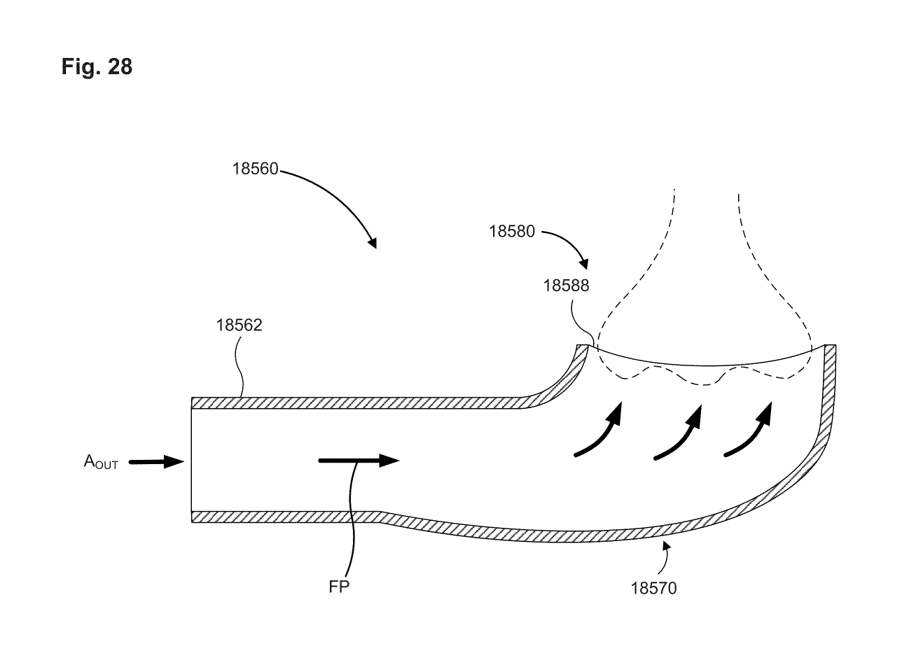

In some embodiments, a face piece can be configured to define an aerosol flow that is surrounded by a secondary gas flow, for example, air or oxygen (O₂) flow. In this manner, the portion of the flow containing the aerosolized particles can be substantially spaced apart from the walls of the face piece during use, thus minimizing impaction and rainout. Referring now to FIG. 29, a face piece 19560 includes an end portion 19562, a plenum portion 19570 and a nasal interface portion 19580. The face piece 19560 can be included in any of the nasal cannula assemblies shown and described herein, for example the nasal cannula assembly 500, and can be used to accomplish any of the methods described herein. The plenum portion 19570 includes a first side wall 19572 that has an inner surface 19573 which defines a flow path FP configured to receive an outlet aerosol A_{OUT} from a supply line, for example the supply line 530, or any other supply line described herein. The plenum portion 19570 includes an end side wall 19574 that defines a curved surface 19575. The nasal interface portion 19580 includes a first delivery protrusion 19582a configured to deliver a first portion of a delivered aerosol A_{DEL1} and a second delivery protrusion 19582b configured to deliver a second portion of a delivered aerosol A_{DEL2} to a second nostril. The plenum portion 19570 and the nasal interface portion 19580 can include features similar to those shown in the plenum portion 5570 and the nasal interface portion 5580 described with respect to the face piece 5560, or any other plenum portions and nasal interface portions described with respect to any of embodiments of the face pieces described herein. The end portion 19562 includes a connection portion 19563 that defines a first opening 19564 configured to be coupleable to the supply line and to receive the aerosol flow. The connection portion 19563 includes a set of fluidic inlets 19569, for examples holes, slots, voids, apertures, or otherwise openings on a side wall of the connection portion 19563. For example, the connection portion 19563 can include 2, 3, 4, or even more fluidic inlets 19569, such that at least two of the fluidic inlets are disposed substantially opposite each other along the periphery of the side wall of the connection portion. A sheathing tube 19590 is coupled to the connection portion 19563, for example via a friction fit, adhesive bond or the like, such that the sheathing tube 19590 surrounds the connection portion 19563. The sheathing tube 19590 can be a hollow tube which is double walled (e.g., includes concentric side walls). The double walls can thereby define a flow path for communicating a sheathing gas flow Aₛ, for example air, or oxygen flow surrounding the aerosol flow. The sheathing tube 19590 also includes one or more fluidic outlets 19592, such that the fluid outlets 19592 of the sheathing tube 19590 are substantially aligned with fluidic inlets 19569. Thus the sheathing tube 19590 can be in fluidic communication with the flow path FP defined by the plenum portion 19570. The sheathing tube 19590 is configured to communicate the sheathing gas flow A_{S} into the connection portion, such that the sheathing gas flow A_{S} surrounds the aerosol flow (i.e., “sheaths” the aerosol flow). The sheathing gas flow Aₛ thereby prevents and/or limits the aerosol flow from contacting an inner surface of the plenum portion 19570 (e.g., the inner surface 19573 or curved surface 19575), and/or an inner surface of the first delivery protrusion 19582a, and/or the second delivery protrusion 19582b. This can reduce and/or prevent impaction of the aerosol on the inner surface 19573 and/or the curved surface 19575 thus reducing rainout and/or sputtering. In some embodiments, the supply line can include a double side wall which defines a flow path for the sheathing gas flow, such that a separate sheathing tube is not required

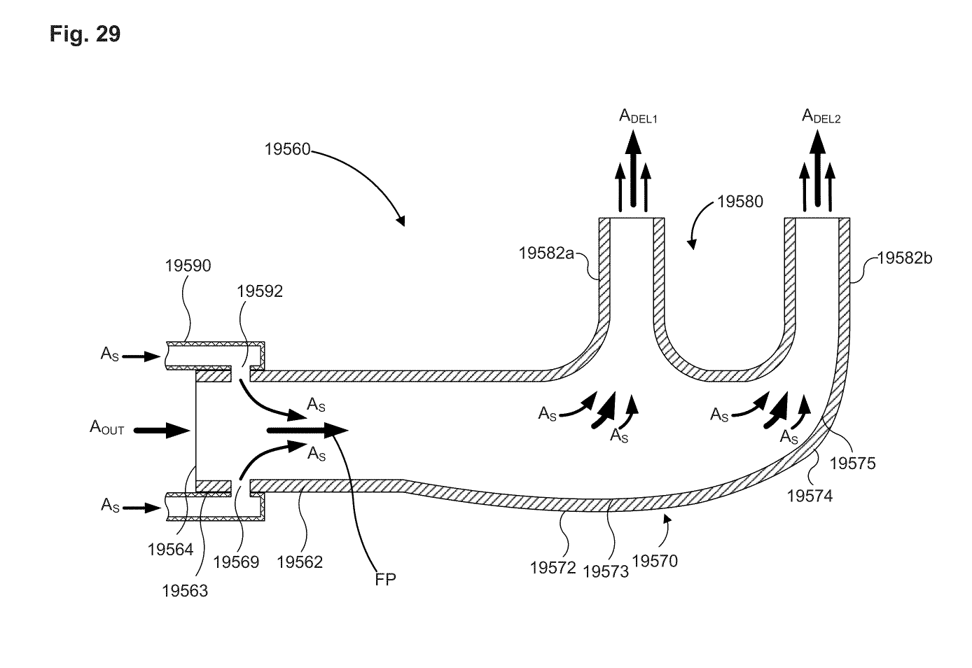

In some embodiments, the sheathed aerosol flow can be produced without including additional features in the connection portion. For example, as shown in FIG. 30, a face piece 20560 includes an end portion 20562, a plenum portion 20570 and a nasal interface portion 20580. The face piece 20560 can be included in any of the nasal cannula assemblies shown and described herein, for example the nasal cannula assembly 500, and can be used to accomplish any of the methods described herein. The end portion 20562, can be substantially similar to the end portion 5562 described with respect to the face piece 5560, or any other embodiments of the face pieces described herein, and is therefore not described in further detail herein. The plenum portion 19570 and the nasal interface portion 19580 can be substantially similar to the plenum portion 5570 and the nasal interface portion 5580 described with respect to the face piece 5560, or any other plenum portions and nasal interface portions described with respect to any embodiments of the face pieces described herein. The end portion 20562 includes a connection portion 20563 which defines an opening 20564 configured to be receive an outlet aerosol A_{OUT} from a supply line 20530. The supply line 20530 has an outer diameter OD_{SL} that is smaller than an inner diameter ID_{CP} of the connection portion 20563. A sheathing tube 20590 is also coupled to the connection portion 20563. An inner diameter of the sheathing tube ID_{ST} is substantially larger than the outer diameter OD_{SL} of the supply line, and is, for example substantially equal to the inner diameter ID_{CP} of the connection portion 20563, such that the sheathing tube 20590 surrounds the supply line 20530. Said another way, the supply line 20530 and the sheathing tube 20590 form concentric tubes, such that the sheathing tube communicates a sheathing gas flow A_{S} surrounding the aerosol flow when the aerosol flow is fluidically communicated into the flow path FP defined by the plenum portion 20570. In some embodiments, the supply line 20530 can be configured to include concentric flow paths such that a separate sheathing tube is not required.

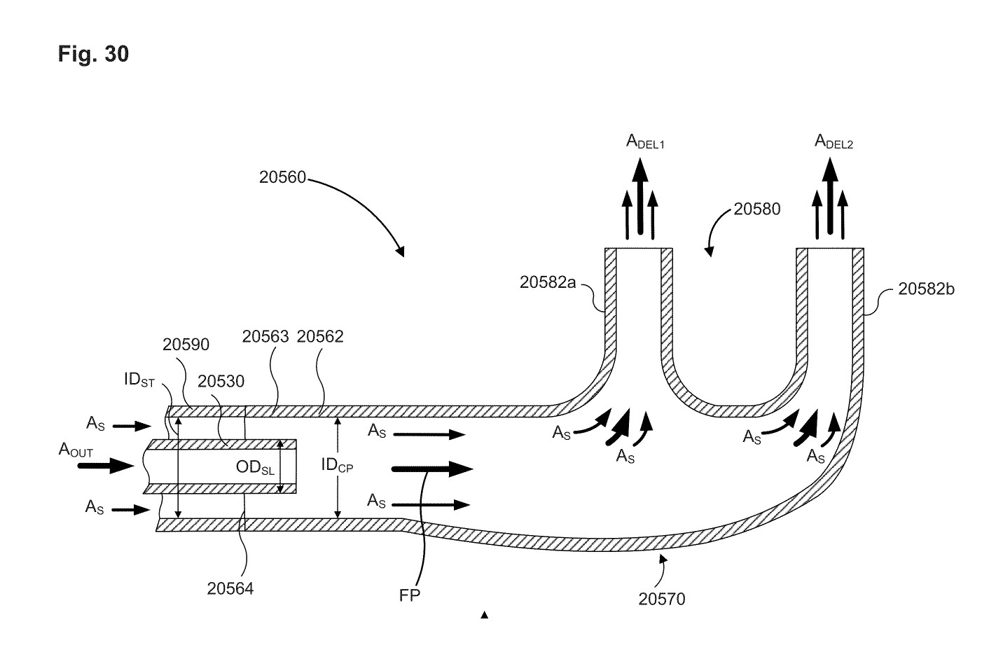

In some embodiments, any of the face pieces described herein can include a mechanism to absorb and/or transport aerosol rainout droplets from an internal surface of the face piece. For example, as shown in FIG. 31, a face piece 21560 includes an end portion 21562, a plenum portion 21570 and a nasal interface portion 21580. The face piece 21560 can be included in any of the nasal cannula assemblies shown and described herein, for example the nasal cannula assembly 500, and can be used to accomplish any of the methods described herein. The end portion 21562, can be substantially similar to the end portion 5562 described with respect to the face piece 5560, and is therefore not described in further detail herein. The plenum portion 21570 can be substantially similar to the plenum portion 5570 described with respect to the face piece 5560, or any other plenum portions described with respect to any embodiments of the face pieces described herein. The nasal interface portion 21580 includes a first delivery protrusion 21582a that has a first inner surface 21584a defining a first nasal flow path 21586a for delivering a first portion of the delivered aerosol A_{DEL1} to a first nostril via a first outlet 21588a. The nasal interface portion 21580 further includes a second delivery protrusion 21582b that has second inner surface 21584b defining a second nasal flow path 21586b for delivering a second portion of the delivered aerosol A_{DEL2} to a second nostril via a second outlet 21588b. The first inner surface 21584a of the first delivery protrusion 21582a and the second inner surface 21584b of the second delivery protrusion 21582b include grooves 21590 defined on the first inner surface 21584a and the second inner surface 21584b, and the inner surface 21573 of the first side wall 21572 of the plenum portion 21570. The grooves 21590 are configured to collect and/or transport aerosol droplets from the first inner surface 21584a or the second inner surface 21584b to the plenum portion 21570 via capillary action. This can prevent aerosol droplets from being delivered to the nostrils of a patient, thereby substantially reducing sputtering. While the first inner surface 21584a and the second inner surface 21584b are shown to have only one groove 21590 each, in some embodiments the first inner surface 21584a, the second inner surface 21584b or any other inner surface of the face piece 21560 can include a plurality of fine grooves. In some embodiments, the grooves 21590 can be textured, for example, the grooves 21590 can include pits and/or dimples.

In some embodiments, the inner surface 21584a of the first delivery protrusion 21582a and/or the inner surface 21584b of the second delivery protrusion 21582b of the face piece 21560 or any of the face pieces described herein, can include features configured to reduce rainout and/or sputtering. For example, the first inner surface 21584a of the first delivery protrusion 21582a and the second inner surface 21584b of the second delivery protrusion 21582b can have protrusions, for example, pillars, cilia like structures, posts, domes or any other suitable structure disposed thereon. The protrusions can restrict the flow of condensed or large aerosol droplets through the first nasal flow path 21586a and/or the second nasal flow path 21586b, thereby limiting sputtering. In such embodiments, aerosol droplets can become entrained into the protrusions and fall back into the plenum portion, for example by the movement or vibrations of the first delivery protrusion 21582a and the second delivery protrusion 21582b. In some embodiments, notches, for example grooves, detents, indents, or pores can be formed on the first inner surface 21584a of the first delivery protrusion 21582a and the second inner surface 21584b of the second delivery protrusion 21582b, disposed near a base of the delivery protrusion. The notches can be configured to retain rainout droplets preventing them from entering the first nasal flow path 21586a and the second nasal flow path 21586b, to limit rainout.

In some embodiments, one or more ledges or shoulders (not shown) can be disposed at the first outlet 21588a and the second outlet 21588b of the first delivery protrusion 21582a and the second delivery protrusion 21582b, respectively. For example, the first outlet 21588a and the second outlet 21588b can be folded in to form the ledge. The ledges can be configured to prevent rainout droplets from being delivered out of the first outlet 21588a of the first delivery protrusion 21582a and the second outlet 21588b of the second delivery protrusion 21582. Rainout droplets can collect beneath the ledges until they reach a critical mass and fall back into the plenum portion 21570. In some embodiments, a membrane that includes a multiplicity of pores can be disposed on the first outlet 21588a and the second outlet 21588b of the first delivery protrusion 21582a and the second delivery protrusion 21582b, respectively. The pores of the membrane can be configured to impede the rainout droplets from passing through but allows the aerosol flow to pass unimpeded through the membrane.

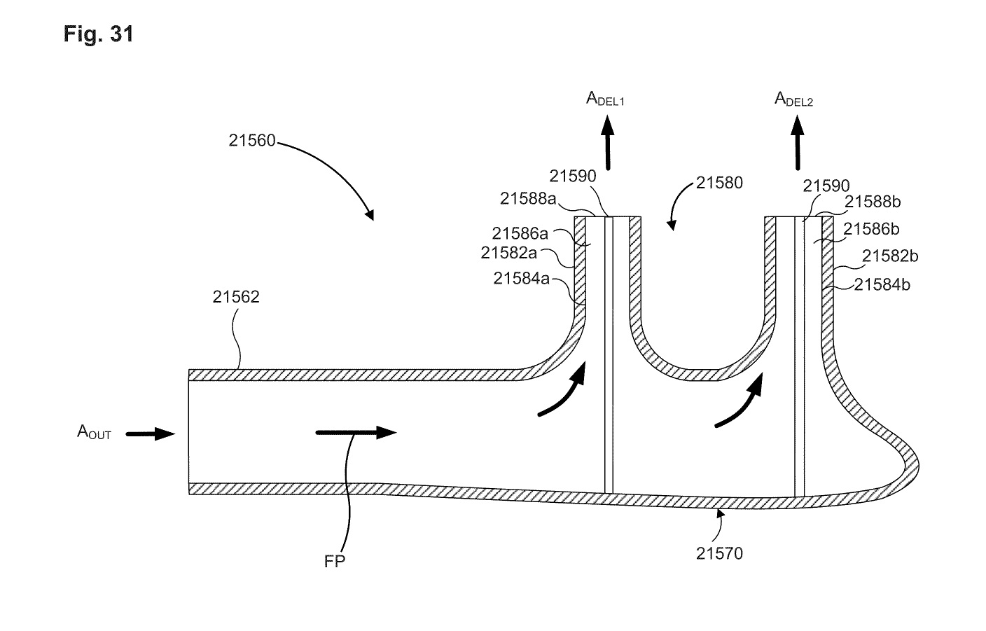

In some embodiments, a face piece can include a reservoir configured to capture aerosol droplets to prevent rainout from being delivered into the nose (i.e., to prevent sputter). Referring now to FIG. 32, a face piece 22560 includes an end portion 22562, a plenum portion 22570 and a nasal interface portion 22580. The face piece 22560 can be included in any of the nasal cannula assemblies shown and described herein, for example the nasal cannula assembly 500, and can be used to accomplish any of the methods described herein. The end portion 22562, can be substantially similar to the end portion 5563 described with respect to the face piece 5560, or any other end portions shown and described with respect to any embodiments of the face piece described herein, and is therefore not described in further detail herein. The nasal interface portion 22580 includes a first delivery protrusion 22582a and a second delivery protrusion 22582b configured to deliver a first portion of the aerosol flow A_{DEL1} and a second portion of the aerosol flow A_{DEL2} to a second nostril. The nasal interface portion 22580 can be substantially similar to the nasal interface portion 5580 described with respect to the face piece 5560, or any other nasal interface portion shown and described with respect to any embodiments of the face piece described herein and is therefore not described herein in further detail. The plenum portion 22570 includes a first side wall 22572 that has a first surface 22573 defining a flow path FP that is configured to receive an aerosol flow from a supply line. The plenum portion 22570 further includes an end side wall 22574 that can have a curved surface 22575. A reservoir 22590 is disposed on a bottom portion of the face piece 22560 such that the reservoir 22590 is in fluidic communication with the flow path of the plenum portion 22570 via fluidic channels 22592. In use, rainout droplets deposited on the first surface 22573 or the curved surface 22575 can be transported through the fluidic channels 22592 (e.g., via capillary action) to the reservoir 22590 where they can be stored and prevented from being delivered to the nostrils of a patient. Similarly stated, this arrangement limits and/or prevents any rainout from “sputtering” into the nostrils via the delivery protrusions. In some embodiments, the fluidic channels 22592 can include a valve or any other mechanism to prevent the captured rainout from being delivered back into the plenum portion 22570, for example if the face piece 22560 is turned upside down.

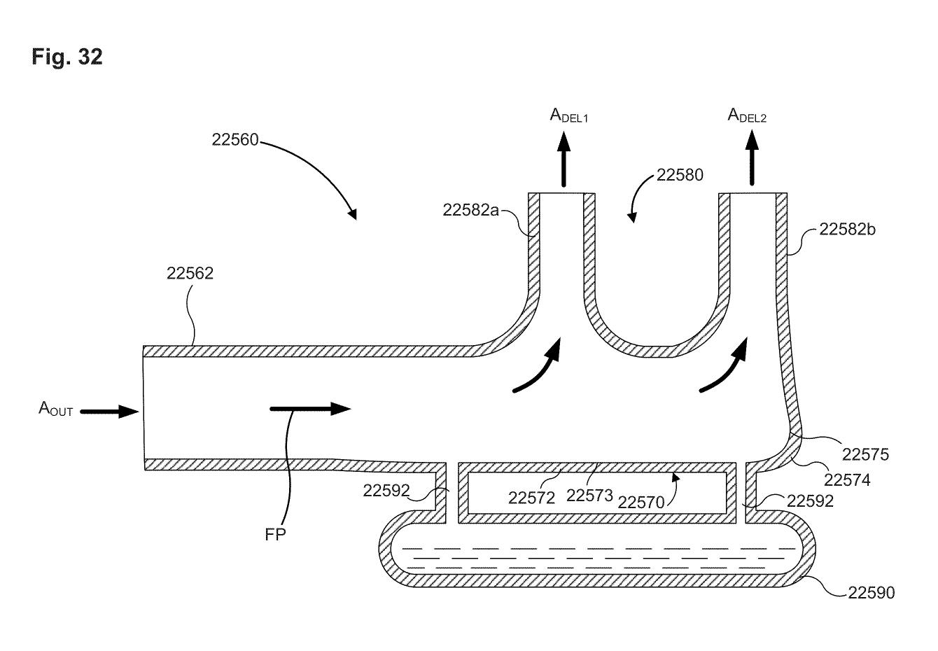

In some embodiments, a face piece can include features, for example wicks, for absorbing and/or transporting rainout droplets, thereby preventing and/or reducing sputter. Referring now to FIG. 33, a face piece 23560 includes an end portion 23562, a plenum portion 23570 and a nasal interface portion 23580. The face piece 22560 can be included in any of the nasal cannula assemblies shown and described herein, for example the nasal cannula assembly 500, and can be used to accomplish any of the methods described herein. The end portion 23562, can be substantially similar to the end portion 5563 described with respect to the face piece 5560, or any other end portions shown and described with respect to any embodiments of the face piece described herein, and is therefore not described in further detail herein. The plenum portion 23570 includes a first side wall 23572 that has an inner surface configured to define a flow path for receiving an outlet aerosol A_{OUT} form a supply line. The plenum portion 23570 also includes an end side wall 23574 that can have a curved surface 23575. The nasal interface portion 22580 includes a first delivery protrusion 22582a having an inner surface 23584a that defines a first nasal flow path 23586a configured to deliver a first portion of the aerosol flow A_{DEL1} to a first nostril via a first outlet 23588a. The nasal interface portion 23580 also includes a second delivery protrusion 23582b having an inner surface 23584b that defines a second nasal flow path 23586b configured to deliver a second portion of the aerosol flow A_{DEL2} to a second nostril via a second outlet 23588b. The plenum portion 23570 and the nasal interface portion 23580 can be substantially similar to the plenum portion 5570 and the nasal interface portion 5580 respectively, described with respect to the face piece 5560, or any other plenum portion or nasal interface portion shown and described with respect to any embodiments of the face piece described herein, and are therefore, not described herein in further detail. A membrane 23590 can be disposed on an inner surface of the face piece 23570, for example an inner surface of a side wall of the end portion 23562, the first surface 23573, the curved surface 23575, and/or the inner surfaces 23584a and 23584b of the first delivery protrusion 23582a and the second delivery protrusion 23582b, respectively. The membrane 23590 can be formed from a liquid absorbent material, for example a desiccant (e.g., silica gel, sodium polyacrylate, clay (bentonite) or calcium chloride), paper, non-woven fiber, cotton wool, surgical cloth, or any other suitable absorbent material, such that the membrane 23590 functions as a wicking layer. The membrane 23590 can thereby, be configured to absorb any rained-out aerosol droplets such that the droplets can transport to a deeper layer. The droplets can thus be prevented from getting entrained in the aerosol flow and being delivered to the nostrils, thereby reducing sputtering. In some embodiments, a liquid absorbent material can be disposed in the plenum portion 23570 or any other portion in an internal volume defined by the face piece 23560 that is operable to absorb the rainout droplets. In some embodiments, the absorbent material can be configured to absorb up to 0.5 ml, 1 ml, 2 ml, 5 ml, or up to 10 ml of the rainout liquid for up to 8 hours of operation of the face piece.

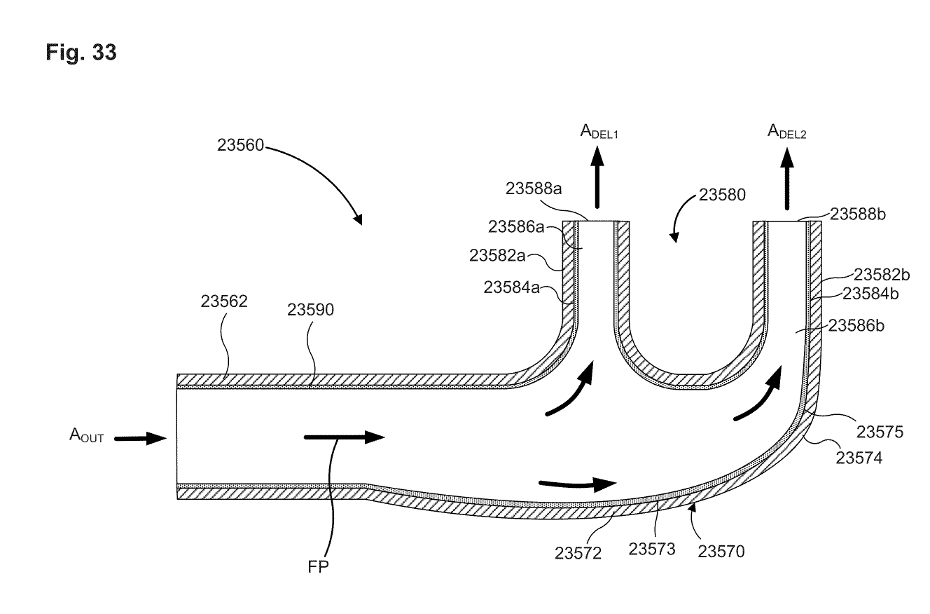

Although the face piece 5560 is shown above as being monolithically constructed and/or constructed from a single material, in other embodiments, any of the face pieces described herein can be constructed from multiple components that are later joined together. For example, in some embodiments, delivery protrusions (or portions thereof) included in a nasal interface portion of a face piece can be formed from an absorbent material. Referring now to FIG. 34, a face piece 24560 includes an end portion 24562, a plenum portion 24570 and a nasal interface portion 24580. The face piece 24560 can be included in any of the nasal cannula assemblies shown and described herein, for example the nasal cannula assembly 500, and can be used to accomplish any of the methods described herein. The end portion 24562 and the plenum portion 24570 can be substantially similar to the end portion 5562 and the plenum portion 5570, respectively, described with respect to the face piece 5560, or any other end portion or plenum portion shown and described with respect to any embodiments of the face piece described herein, and are therefore not described in further detail herein. The nasal interface portion 24580 includes a first delivery protrusion 24582a having an inner surface 24584a that defines a first nasal flow path 24586a configured to deliver a first portion of the aerosol flow A_{DEL1} to a first nostril via a first outlet 24588a. The nasal interface portion 24580 also includes a second delivery protrusion 24582b having an inner surface 24584b that defines a second nasal flow path 24586b configured to deliver a second portion of the aerosol flow A_{DEL2} to a second nostril via a second outlet 24588b. Each of the first delivery protrusion 24582a and the second delivery protrusion 24582b can be formed from an absorbent material, for example paper, cardboard, or any other suitable absorbent material, such that rainout droplets can be absorbed by the first delivery protrusion 24582a and the second delivery protrusion 24582b and prevented from being delivered to the nostrils of a patient. In some embodiments, an absorbent material, for example a wicking material, can be disposed on a portion of the inner surfaces 24584a and 24584b of the first delivery protrusion 24582a and the second delivery protrusion 24582b. In such embodiments, a portion of the absorbent material can also be disposed in the plenum portion 24570.

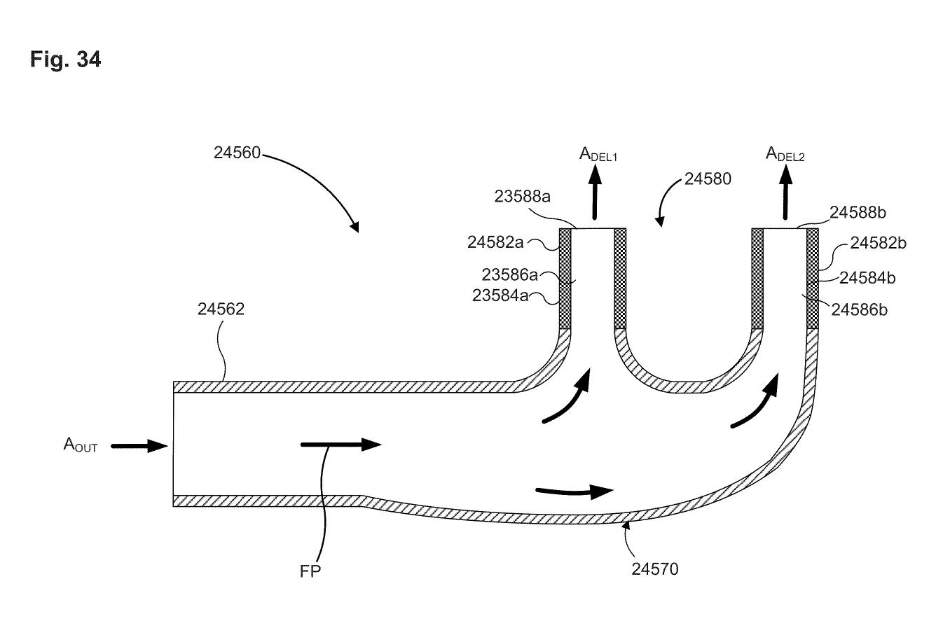

In some embodiments, an absorbent material, for example a wick can be disposed in a second end portion of a face piece to absorb rainout droplets. Referring now to FIG. 35, a face piece 25560 includes a first end portion 25562, a plenum portion 25570, a nasal interface portion 25580, and a second end portion 25566. The face piece 25560 can be included in any of the nasal cannula assemblies shown and described herein, for example the nasal cannula assembly 500, and can be used to accomplish any of the methods described herein. The nasal interface portion 25580 includes a first delivery protrusion 25582a and a second delivery protrusion 25582b. The first end portion 25562 and the nasal interface portion 25580 can be substantially similar to the first end portion 5562 and the 5563 described with respect to the face piece 5560, or any other first end portion or nasal interface portion shown and described with respect to any embodiments of the face piece described herein, and are therefore, not described herein in further detail. The plenum portion 25570 includes a first side wall 25572 that has an inner surface 25573. The inner surface 25573 defines a flow path FP configured to receive an outlet aerosol A_{OUT} from a supply line, for example the supply line 530 or any other supply line described herein, coupled to the first end portion 25562. The second end portion 25566 is disposed opposite the first end portion 25562 and defines an inner volume. An absorbent material 25590, for example a wick is disposed in the inner volume defined by the second end portion 25566, such that the absorbent material 25590 forms a plug that blocks an aerosol flow from entering into the second end portion 25566 from the plenum portion 25570. A side wall 25592 of the absorbent material 25590 blocks the aerosol flow from entering the second end portion 25566 and can be configured to have a curved surface that directs a portion of the aerosol flow towards the second delivery protrusion 25582b, or define a flow restriction in the flow path. The absorbent material 25590 is configured to absorb rainout droplets and wick it away from the plenum portion 25570 such that rainout and/or sputtering is minimized and/or reduced. The absorbent material 25590 can include any suitable absorbent material, for example paper, cardboard, cotton wool, non-woven fiber, any other fabric, silica gel, any suitable desiccant or absorbent material. In some embodiments, a paper wick can be disposed contiguous with an inner surface and along the entire circumference of the second end portion 25566 (i.e., a full wick). In some embodiments, a paper wick can be disposed along only a portion of the circumference of the second end portion 25566 (i.e., a partial wick), for example ½ of the circumference or even ¼ of the circumference. In such embodiments, the wick can be in fluidic communication with a rainout sink, for example a paper plug, which can be disposed on the second end portion 25566. In some embodiments, a strip of a wicking material can be disposed on an inner surface of the delivery protrusions 25582a and/or 25582b, and/or an inner surface 25573 of the plenum portion. In such embodiments, the strip of wicking material can be a straight, curved, bent, or spiral strip.

In some embodiments, any of the absorbent materials or wicking materials described herein can be configured to absorb up to 0.5 ml, 1 ml, 2 ml, 5 ml, or up to 10 ml of the rainout liquid for up to 8 hours of operation of the face piece.

In some embodiments, a surfactant can be included in the aerosol formulation such that the surfactant prevents the assimilation of the droplets of the aerosol flow into larger droplets (e.g., droplets having a size and/or a distribution of greater than about 5 μm), that can cause rainout and/or sputtering. Suitable surfactants can include, for example, surfactants added for treatment for neonatal patients. In some embodiments, an inner surface of a face piece, for example, the face piece 5560 or any other face piece described herein can be coated with a soluble coating, for example, a surfactant. In this manner, the face piece and/or any other portion of the cannula assembly can include a portion of the composition delivered. The coating can dissolve in the aerosol flow to alter the surface energy of the droplets of the aerosol flow (e.g., reduce a contact angle of the aerosol droplets), thereby reducing rainout and/or sputtering.

In some embodiments, a face piece can include “active” mechanisms for controlling and/or reducing rainout and/or sputter. For example, in some embodiments, a face piece can include temperature control features, for example micro heaters embedded in a side wall of a face piece (e.g., the face piece 5560 or any other face piece described herein) configured to heat the face piece and evaporate rainout droplets. In some embodiments, a rainout clearing mechanism can be included in the face piece to clear rainout. Such a mechanism can include, for example, an actuator, an arm, a piston, a flap, a protrusion, a gate between the delivery protrusions, a floating auto shutoff valve or any other feature, disposed in a first end portion or a second end portion of face piece, that can be included in a plenum portion of the face piece to physically clear the rainout. In some embodiments, a face piece can include an automatic drain which can be opened by a time to eject collected rainout after a predetermined period of time.

In some embodiments, the aerosol flow into any of the face pieces described herein can be characterized by a pulsatile flow. Said another way, the aerosol can be communicated into a face piece, for example, the face piece 5560 or any other face piece described herein, in a series of pulses. This arrangement may produce lower particle size selections and/or more efficient particle selection compared to non-pulsative entrainment fluids of the same average flow rate. The source of such pulsatile flow may be, but is not limited to, a diaphragm pump, a peristaltic pump, a rotary vane pump or compressed gas with an actuated valve producing the oscillatory pattern connected in series and upstream of the entrainment chamber. In some embodiments, the pulsatile flow can be configured to deliver a series of forward pulsed followed by a back pulse, for example, to periodically draw out aerosol droplets deposited in the plenum portion or the nasal interface portion of the face piece.

Any supply line can be used in conjunction with any of the face pieces or nasal cannula assembly described herein. In some embodiments, the supply line, for example the supply line 530 can be coupled to a connection portion of a first end portion of a face piece, for example, the connection portion 5563 of the first end portion 5562 of the face piece 5560. In some embodiments, the supply line can be coupled to a bifurcation piece, for example the bifurcation piece 534 described with respect to FIG. 1, and two face piece tubes (e.g., the face piece tube 535a and the face piece tube 535b) can be used to communicate the aerosol flow to a face piece. In such arrangements, the face piece can be a bilateral configuration (i.e., configured to receive the aerosol flow from two different inlets) or can include independent plenums and nasal protrusions (one for each nostril, each being coupled to one outlet of the bifurcation piece).

The supply line for any of the nasal cannula assemblies shown herein can have any suitable length and diameter. For example, a supply line can have a length in the range from about 4 foot to about 7 foot. For example, the supply line can have a length of about 4 foot, 4.5 foot, 5 foot, 5.5 foot, 6 foot, 6.5 foot, 7 foot, or even higher, inclusive of all ranges therebetween. In some embodiments, the supply line can have an inner diameter of about 4.6 mm and an outer diameter of about 6 mm. The inner diameter of the supply line can be configured to be substantially similar to an inner diameter of a first end portion of a face piece (e.g., the first end portion 5562 of the face piece 5560). This can ensure that the aerosol flow can be delivered from the supply line to a plenum portion of a face piece (e.g., the plenum portion 5570 of the face piece 5560) without encountering any sharp bends, steps, impediments, hurdles, or other flow obstacles that can increase generate vortices or eddies in the aerosol flow. Said another way, the supply lines and the face pieces disclosed herein can, in some embodiments, be cooperatively configured such that the aerosol flow can flow smoothly into the plenum portion of the face piece thereby reducing rainout. In some embodiments, the outer diameter of any of the supply lines described herein can be in close tolerance with the inner diameter of a connection portion (e.g., the connection portion 5563) such that the supply line can be friction fit into the connection portion. In some embodiments, a distal end of the supply line can include threads, or a luer lock assembly, for coupling to the connection portion. In some embodiments, the supply line can be clamped onto the face piece, or affixed thereto with an adhesive. In some embodiments, an inner diameter of a supply line can be selected to reduce gravitational sedimentation of the aerosol particles, which can accumulate as rainout in the face piece. For example, sedimentation may be reduced by decreasing the diameter of the supply line included in a cannula assembly, for example the cannula assembly 500, to produce an increased velocity with which the aerosol travels through the cannula assembly. Moreover, methods according to an embodiment can include increasing the airflow from 1 L/min, to 2 L/min, to 3 L/min, to 4 L/min, and/or higher.

In some embodiments, any of the supply lines described herein can have a smooth bore. In other embodiments, any of the supply lines described herein can include a finned bore which can serve to minimize kinking in the supply line. A finned bore can, however cause a substantial increase in the internal surface area of the supply line which can lead to increased rainout within the supply line. In such embodiments, the flow rate and/or internal diameter of the supply line can be adjusted accordingly to minimize, reduce, and/or otherwise eliminate the rainout.

Any of the supply lines described herein can be made from any suitable material to reduce rainout. Suitable materials can include, for example, plastics, silicone, Teflon, rubber, polymer, or any other flexible material. In some embodiments, an inner surface of the supply line can be coated with an hydrophobic material such as, for example silica nano coating, precipitated calcium carbonate, zinc oxide polystyrene nano-composite, manganese oxide polystyrene nano-composite, oils, lipids, fats, NeverWet™, P2i™, Aculon™, Lotus Leaf coatings, or any other hydrophobic coatings or combination thereof.

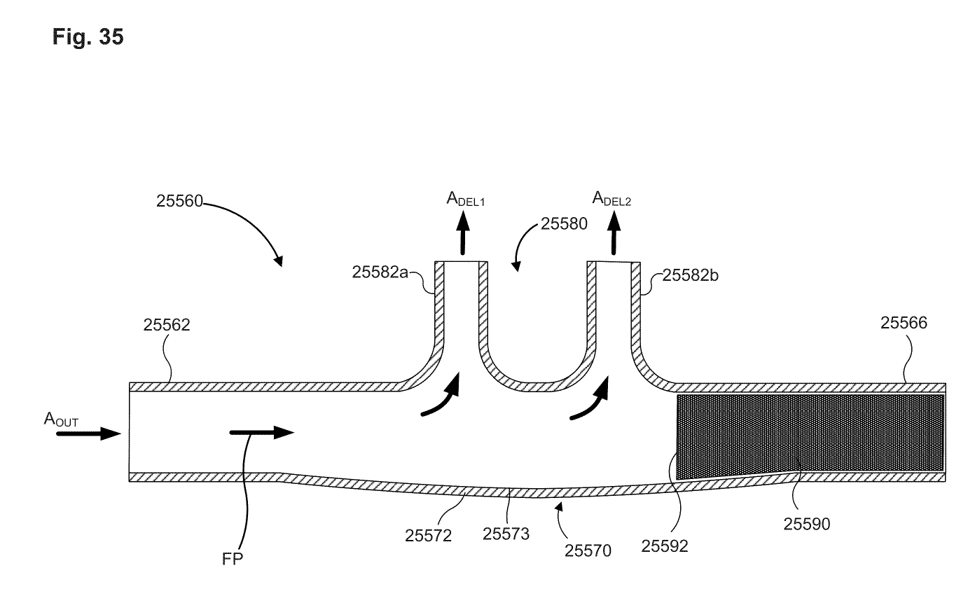

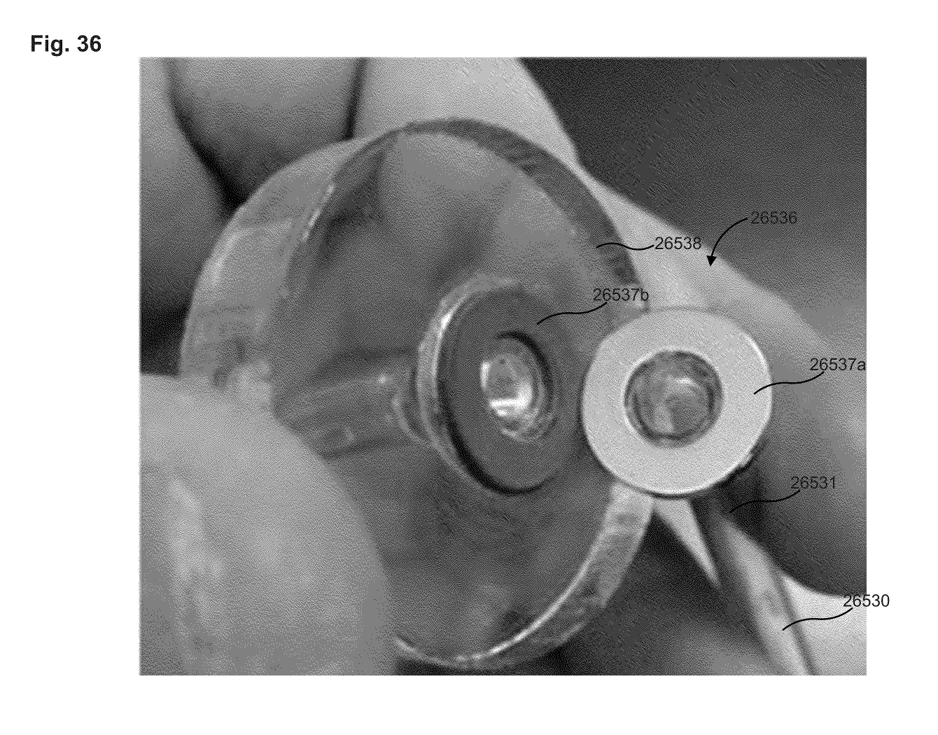

Although the coupling mechanism described herein (e.g., to couple a supply line to a face piece and/or an aerosol preparation assembly) have been described primarily as being press fit arrangements, in other embodiments, any suitable mechanism for coupling a supply can be used. In some embodiments, a proximal end of a supply line can be coupled to an aerosol preparation assembly or a face piece via a magnetic coupling mechanism. Referring now to FIGS. 36 and 37A-B, a coupling mechanism 26536 can include a first magnet 26537a coupled to a proximal end 26531 of a supply line 26530. The coupling assembly 26536 further includes a second magnet 26537b disposed on a grip 26538, which, in this instance, is coupled to an aerosol preparation assembly tube 26102. The second magnet 26537b can have an opposite polarity to the first magnet 26537a, such that the two magnets aid, guide, lock, align, and/or otherwise enhance the coupling of the aerosol preparation assembly tube 26102 to the supply line 26530. The aerosol preparation assembly tube 26102 can be coupled to an aerosol preparation assembly, for example the aerosol preparation assembly 100, or to a face piece (e.g., face piece 5560). The first magnet 26537a and the second magnet 26537b define a lumen which is fluid communication with the lumen of the supply line tube 26530 and the aerosol preparation assembly tube 26102, respectively. In a first configuration shown in FIG. 37A, the first magnet 26537a and the second magnet 26537b can be uncoupled. A user can bring the first magnet 26537a and the second magnet 26537b close together, such that the magnets attract each other and are reversibly coupled together in a second configuration shown in FIG. 37B. In this manner, the supply line 26530 can be reversibly coupled to the aerosol preparation assembly. A seal, for example a rubber gasket can be disposed on at least one of the first magnet 26537a or the second magnet 26537b to prevent the aerosol flow from leaking from the coupling mechanism 26536. The coupling mechanism 26530 can be configured, such that an inner wall of the supply line 26530 and an inner wall of the aerosol preparation assembly tube 26102 define a substantially continuous and smooth surface. The smooth surface ensures that the flow path of the aerosol flow is free of sudden expansions, impediments, or obstacles thereby preventing sudden change in velocity and minimizing rainout.

Although shown and described without temperature control, in some embodiments, any of the nasal cannulas described herein, for example, the nasal cannula assembly 500, can include a temperature control mechanism. For example, the nasal cannula assembly can include a heater to reduce condensation and/or to provide an aerosol flow to a user at a predetermined temperature. In such embodiments, a temperature sensors (e.g., a thermocouple, or a thermistor), and a feed back control loop can be included in the nasal cannula assembly to ensure that the aerosol is maintained at a desired temperature.

Any of the face pieces described herein can be made from a soft and flexible material, for example, silicone, silicone rubber, polydimethylsiloxane, neoprene, latex, rubber, polystyrene, polybutenes, plastics, Teflon, polymers, fiber or metal reinforced polymers or any other suitable material.

In some embodiments, the manufacturing process can also affect the rainout and/or sputter performance of a face piece, for example, the face piece 5560, 6560, or any other face piece described herein. For example, as shown in the experimental results below, a first face piece can be formed using a first manufacturing process (e.g., stereolithography) and can produce a first rainout, for example, of less than about 1% (e.g., about 0.5%, or about 0.1%) of the amount of liquid conveyed therethrough. A second face piece which can be substantially similar to the first face piece in material(s), shape, size, and/or geometry can be formed using a second manufacturing process (e.g., molding). The second face piece can, for example, produce a second rainout different than the first rainout. For example, the second rainout can be less than about 10%, for example, less than about 9%, about 8%, about 7%, about 6%, about 5%, about 4%, about 3%, or less than about 2% Without being bound by any particular theory, this difference can be a result of the different surface energies or any other surface property (e.g., hydrophobicity/hydrophilicity) of the first face piece relative to the second face piece imparted as a result of the different manufacturing process. In such embodiments, modifications to the manufacturing process can be made or post processing can be performed on the manufactured face piece to reduce, minimize, or otherwise eliminate rainout.

For example, in some embodiments, a molding process used for manufacturing a face piece can exclude the use of release agents such as, for example, oils, waxes, diluted silicone, silanes, fluorocarbons, or any other mold release agent that can be used as a release agent for molding a face piece formed from any of the materials described herein. In some embodiments, a molding process for forming a face piece can include operations to modify the surface energy of the molded face piece such that the molded face piece has a reduced rainout and/or sputter performance in use. In some embodiments, the rainout performance of a face piece (e.g., a molded face piece) can be reduced by post-processing the face piece via any suitable process that can modify the surface energy of the face piece and reduce rainout. For example, the manufactured face piece can be treated with a UV (ultra violet) plasma, oxygen plasma, electric arc, or any other suitable post treatment process. In other embodiments, any of the face pieces described herein can include a surface coating of the types described herein.

In some embodiments, a nasal cannula assembly can be coupled to a mounting assembly configured to mount the cannula assembly on a face, head, or ear of the user, such that a face piece included in the nasal cannula assembly is disposed in proximity of the nostrils of the user. The mounting assembly can be ergonomically designed such that the user can wear the mounting assembly for extended periods of time without any discomfort or impediment to normal movements of the user.

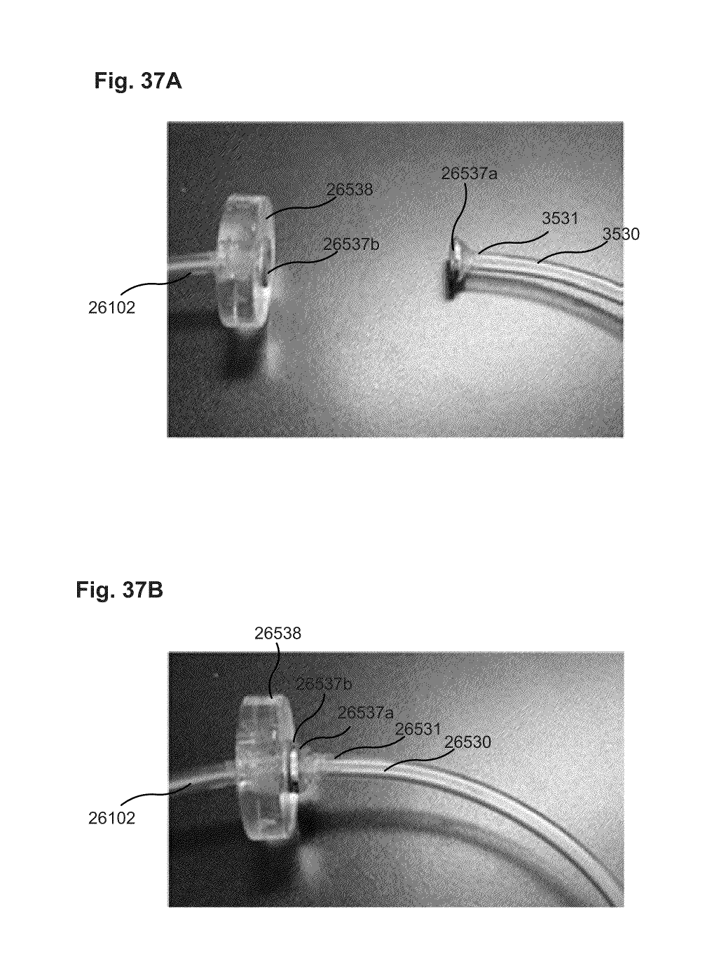

In some embodiments, one or more face piece tubes included in a nasal cannula assembly can be used to mount the nasal cannula assembly. For example, as shown in FIG. 38, a mounting assembly 1700 can include a first face piece tube 1735a and a second face piece tube 1735b (collectively referred to as “the face piece tubes 1735”) that are coupled to a face piece 5560 as described herein. The mounting assembly 1700 can be used with any of the nasal cannula assemblies and/or face pieces described herein, and the face piece 5560 is depicted only as a non-limiting example. A distal end of the first face piece tube 1735a can be coupled to a connection portion 5563 of the face piece 5560, and configured to deliver an aerosol flow to the face piece 5560. The second face piece tube 1735b can be a “dummy” tube, for example a solid tube that does not include a lumen. A distal end of the second face piece tube 1735b can be coupled to a mounting portion 5567 of the face piece 5560. The first face piece tube 1735a and the second face piece tube 1735b can be soft and flexible, such that the first face piece tube 1735a can be looped around a first ear of the user and the second face piece tube 1735b can be looped around a second ear of the user. At least a portion of each face piece tube 1735a, 1735b is coupled to a retainer 1740. For example, the face piece tubes 1735a, 1735b can be slidably disposed in a set of grooves defined by the retainer 1740, such that the user can slide the retainer over the face piece tubes 1735 towards or away from a chin of the user. This can allow the user to tighten and thereby secure the face piece tubes 1735 under the chin, for example, to allow positioning of the face piece 5560 in proximity of the nostrils of the user.

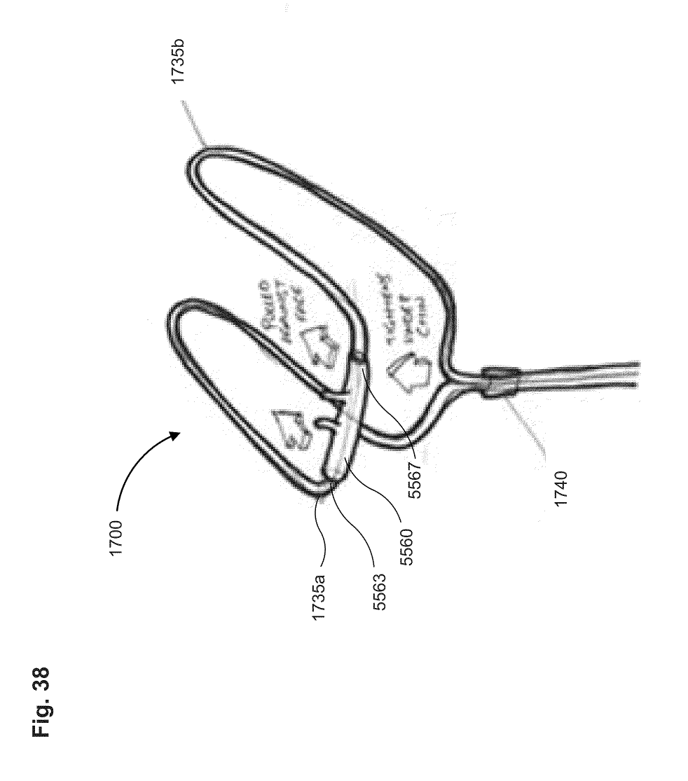

In some embodiments, a mounting assembly for mounting a nasal cannula assembly on the face of a user can include a cheek brace. Referring now to FIG. 39, a mounting assembly 2700 can include a cheek brace 2740. The mounting assembly 2700 can be used with any of the nasal cannula assemblies and/or face pieces described herein. As shown in, the face piece 5560, as described before herein, can be disposed on a front portion 2742 of the cheek brace 2740. The front portion 2742 is configured to be disposed below the nostrils of the user. The cheek brace 2740 defines a curved surface configured to conform to the contours of the face of the user, such that a side portion 2744 of the cheek brace 2740 can be disposed on the cheek of the user. A releasable adhesive can at least partially coat an inner surface 2745 of the cheek brace 2740, such that the cheek brace 2740 can be releasably disposed on the face of the user. Example adhesives include, but are not limited to, acrylate based medical adhesives of the type commonly used to affix medical devices such as bandages to skin. In some embodiments, the cheek brace 2740 can be made from hard but flexible material, for example, plastics. In some embodiments, the cheek brace 2740 can be padded, for example, with foam. In some embodiments, an outer surface of the cheek brace 2740 can be covered with a soft and air breathable material, for example, a fabric (e.g., silk, wool, cotton, polyester, nylon, or any suitable fabric).

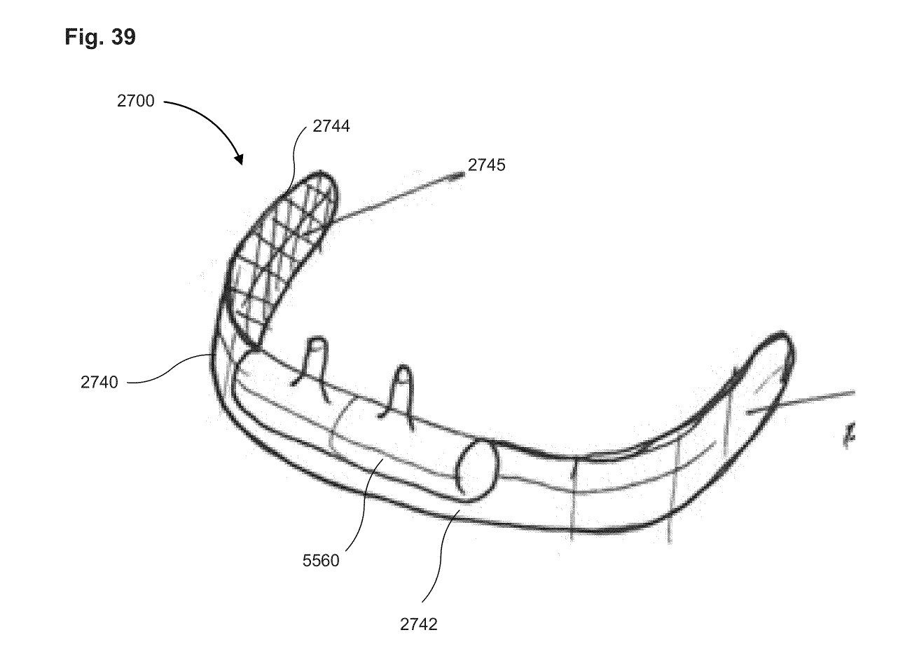

In some embodiments, a mounting assembly for mounting a nasal cannula assembly on the face of a user can include a head gear that is distinct from the supply tube and/or face piece tube. Referring now to FIG. 40, a mounting assembly 3700 can include a head gear 3740 configured to be worn on the head of a user. The mounting assembly 3700 can be used with any of the nasal cannula assemblies described herein, for example the nasal cannula assembly 500. The head gear 3740 includes a first arm 3742a and a second arm 3742b, which are configured to brace the head of a user. A first eye brace 3744a and a second eye brace 3744b (collectively referred to as the eye braces 3744) are coupled to the first arm 3742a and the second arm 3742b respectively. The first arm 3742a and the second arm 3742b are joined together at a base 3746. The head gear 3740 can be monolithically formed from a flexible material, for example a plastic.

Because the eye braces 3744 are not coupled together, a user can flex each of the first arm 3742a and the second arm 3742b outwards such that the head gear 3740 can be easily disposed on the head of the user. The eye braces 3744 are configured to cover the eyes of the user when the nasal cannula is positioned to deliver the aerosol flow to the nostrils of the patient. Said another way, the head gear 3740 can be used as an eye mask by the user. For example, because the nasal cannula assemblies can provide for a long treatment duration, the user can wear the mounting assembly 3700 while sleeping. An inner surface of the first arm 3742a and the second arm 3742b can include a coupling mechanism configured to reversibly secure a supply line 5530 and/or one or more face piece tubes. Examples of suitable coupling mechanisms include, grooves, notches, indents, hooks, elastic bands, clips, or any other suitable coupling mechanism.

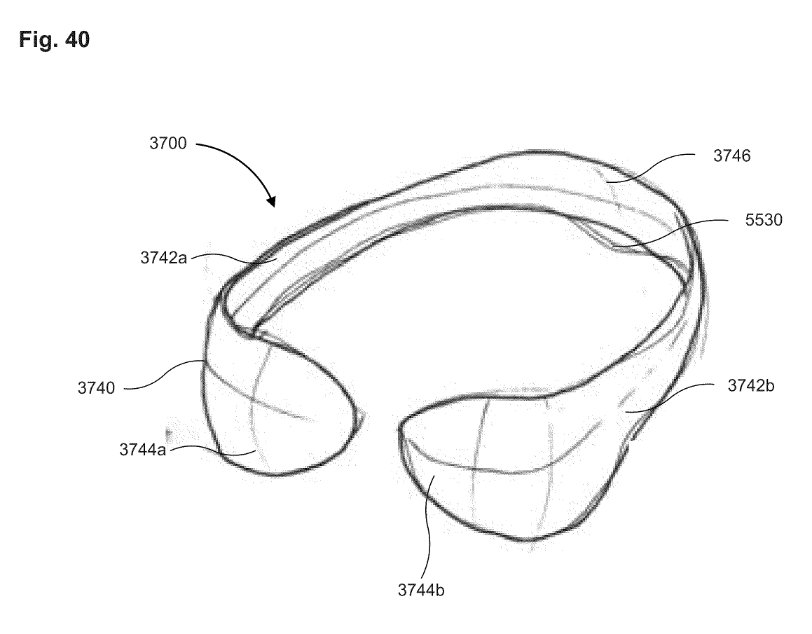

In some embodiments, a head gear can have a single eye brace. For example, referring now to FIG. 41, a mounting assembly 4700 includes a head gear 4740 which has the face piece 5560, as described before herein, mounted thereto. The mounting assembly 4700 can be used with any of the nasal cannula assemblies described herein, for example the nasal cannula assembly 500. The head gear 4740 includes a head brace portion 4742 and an eye brace portion 4744. The head gear 4740 is configured such that when a user disposes the head gear 4740 on the head of the user, the head brace portion 4742 surrounds the head of the user, the eye brace portion 4744 covers the eyes of the user, and the face piece 5560 is disposed in proximity of the nares of the user. An inner surface of the head brace portion 4742 can include grooves, notches, indents, hooks, elastic bands, clips, or any other suitable coupling mechanism, to secure a supply line 5530, a first face piece tube 5535a and/or a second face piece tube 5535b. The first face piece tube 5535a and the second face piece tube 5535b are coupled to the face piece 5560, such that securing the first face piece tube 5535a and the second face piece tube 5535b to the head gear 4740 mounts the face piece 5560 on the head gear.

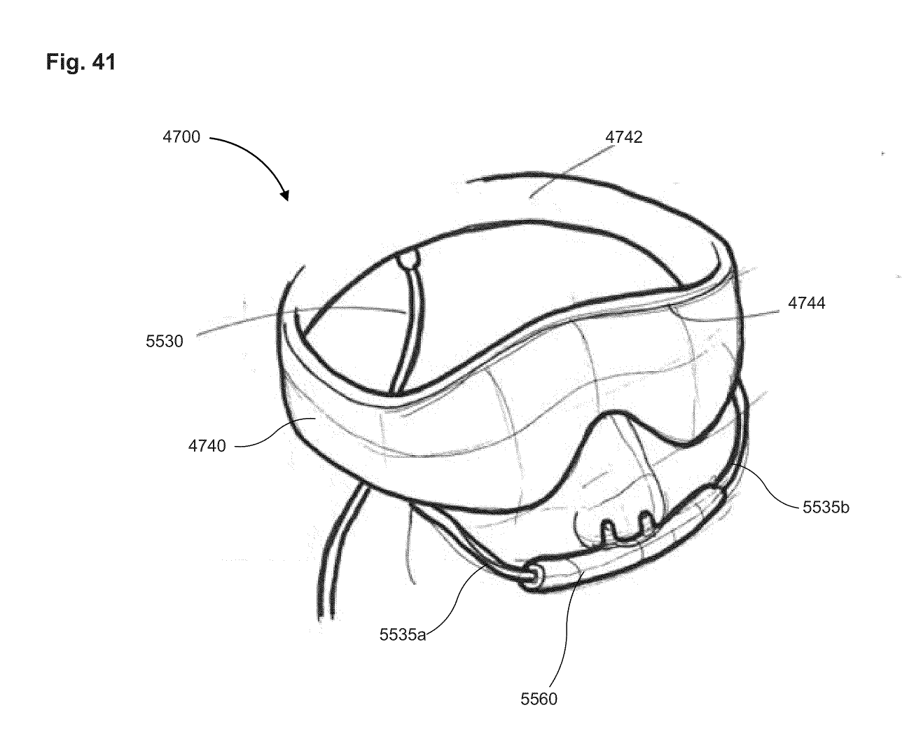

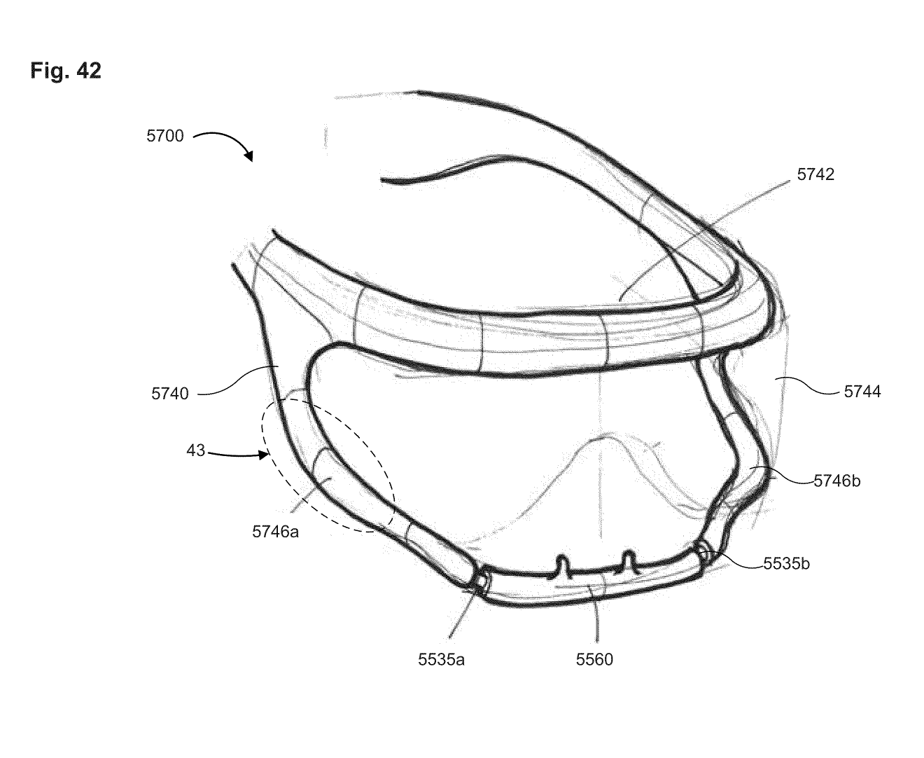

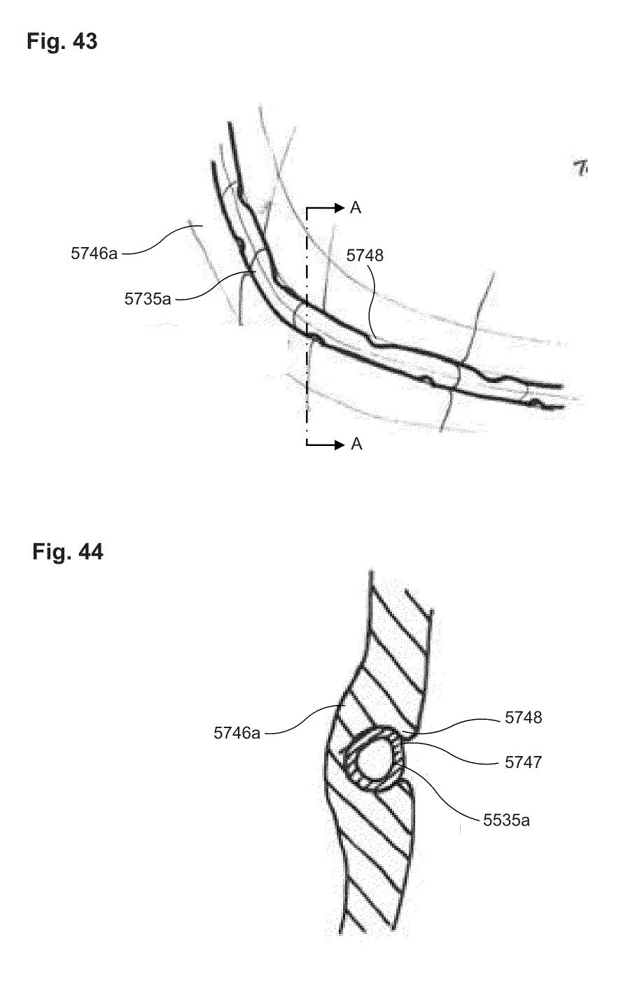

In some embodiments, a head gear can include a portion for mounting a face piece tube. Referring now to FIG. 42-44, a mounting assembly 5700 includes a head gear 5740 which has the face piece 5560, as described before herein, mounted thereto. The mounting assembly 5700 can be used with any of the nasal cannula assemblies described herein, and the face piece 5560 is presented as a non-limiting example. The head gear 5740 includes a head brace portion 5742 configured to be disposed on the head of a user. The head gear 5740 further includes a first face piece tube mount 5746a and a second face piece tube mount 5746b, which are configured to mount a first face piece tube 5535a and the second face piece tube 5535b. FIG. 43 shows an enlarged view of an inner surface of the portion of the first face piece tube mount 5746a shown by the region identified as region 43 in FIG. 42. FIG. 44 shows a cross-section view of FIG. 43 along the line AA. The inner surface of the first face piece tube mount 5735a (and the second face piece tube mount 5735b) includes a groove 5747 and a multiplicity of ledges 5748 on a rim of the groove 5747. The groove 5747 can be sized and shaped such that the first face piece tube 5735a can be disposed into the groove 5747. The ledges 5748 can be flexible such that the first face piece tube 5735a can be push-fit into the groove and removably coupled to the first face piece tube mount 5746a. Furthermore, each of the first face tube mount 5746a and the second face piece tube mount 5746b can be curved to conform to the contours of the users face.

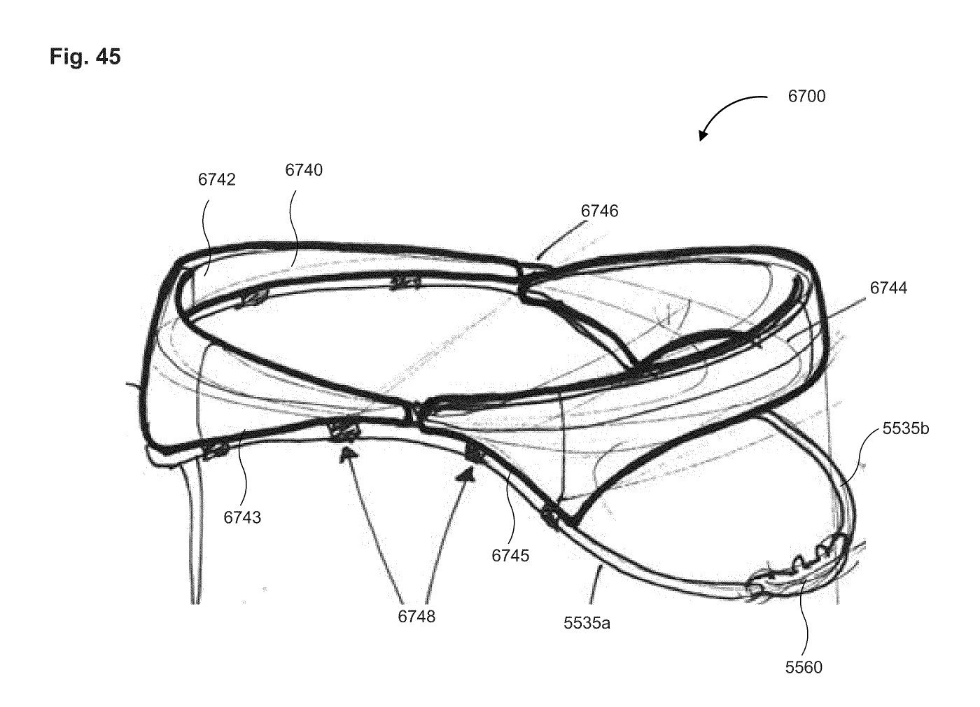

In some embodiments, a head gear can include clamps or bands to secure a face piece tube. Referring now to FIG. 45, a mounting assembly 6700 includes a head gear 6740 which has the face piece 5560, as described before herein, mounted thereto. The mounting assembly 6700 can be used with any of the nasal cannula assemblies described herein, for example the nasal cannula assembly 500. The head gear 6740 includes a first portion 6742 and a second portion 6744. The first portion 6742 is configured to brace a back of the head of a user, and the second portion 6744 is configured to brace a forehead of the user. The first portion 6742 and the second portion 6744 are coupled together at an elastic joint 6746. The elastic joint can be, for example, an elastic band, a rubber band, or any other elastic joint. In some embodiments, the first portion 6742 and the second portion 6744 can be pivotably coupled to each other. The first portion 6742 and the second portion 6744 can be formed from or covered with a soft padded material so that the head gear 6740 can be worn comfortably by the user. A set of securing bands 6748 are disposed on a first side 6743 of the first portion and a second side 6745 of the second portion 6744. The securing bands 6748 are configured to secure a first face piece tube 5535a and a second face piece tube 5535b which are coupled to the face piece 5560. In this manner, coupling the first face piece tube 5535a and the second face piece tube 5535b mounts the face piece 5560 on the head gear 6740, such that when the user wears the head gear 6740, the face piece 5560 is disposed in proximity to nares of the user.

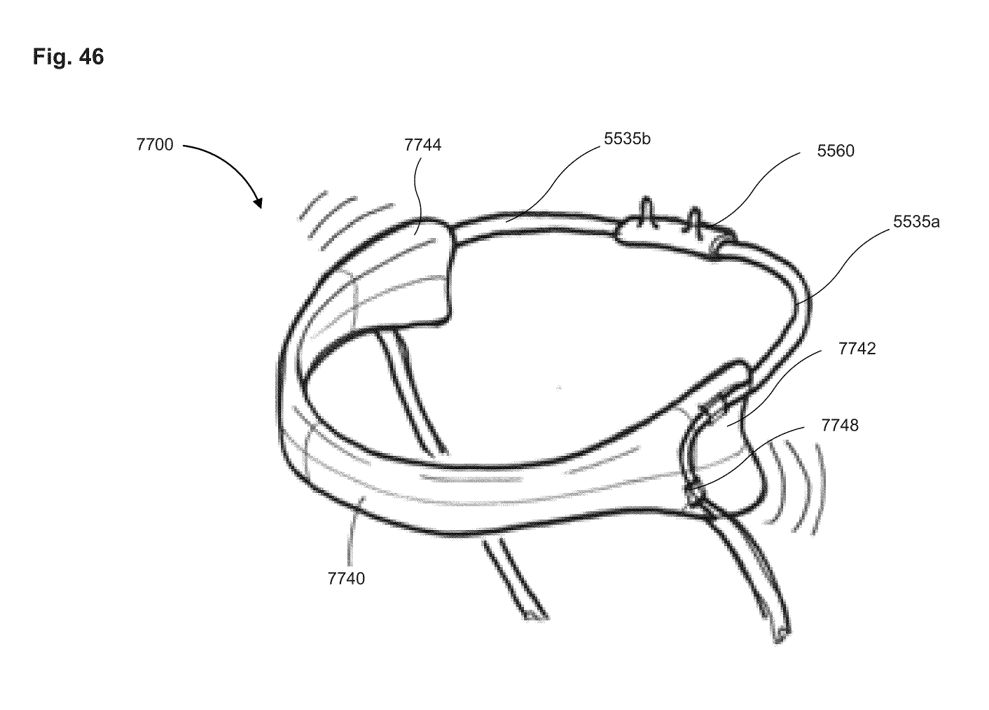

In some embodiments, a head gear included in a mounting assembly can be configured to only brace a back of a head of a user. Referring now to FIG. 46, a mounting assembly 7700 includes a head gear 7740 which has the face piece 5560, as described before herein, mounted thereto. The mounting assembly 7700 can be used with any of the nasal cannula assemblies described herein, for example the nasal cannula assembly 500. The head gear 7740 can be formed from a rigid but elastic material, for example a plastic. The head gear 7740 can be covered with a soft padded material, for example foam so that the head gear 7740 can be worn comfortably by a user. The head gear 7740 is configured to be mounted on a back of a head of a user, such that a first portion 7742 and a second portion 7744 of the head gear 7740 braces the sides of the head of the user. The user can exert an outwardly force on each of the first portion 7742 and the second portion 7744 such that the head gear 7740 flexes and can easily be worn or taken off by the user. A set of securing bands 7748 are disposed on each of the first portion 7742 and the second portion 7744. The securing bands 7748 can include elastics hoops, clamps, or any other securing mechanism. The securing bands 7748 are configured to secure a first face piece tube 5535a and a second face piece tube 5535b which are coupled to the face piece 5560, such that the first face piece tube 5535a and the second face piece tube 5535b loop around the ears of the user.

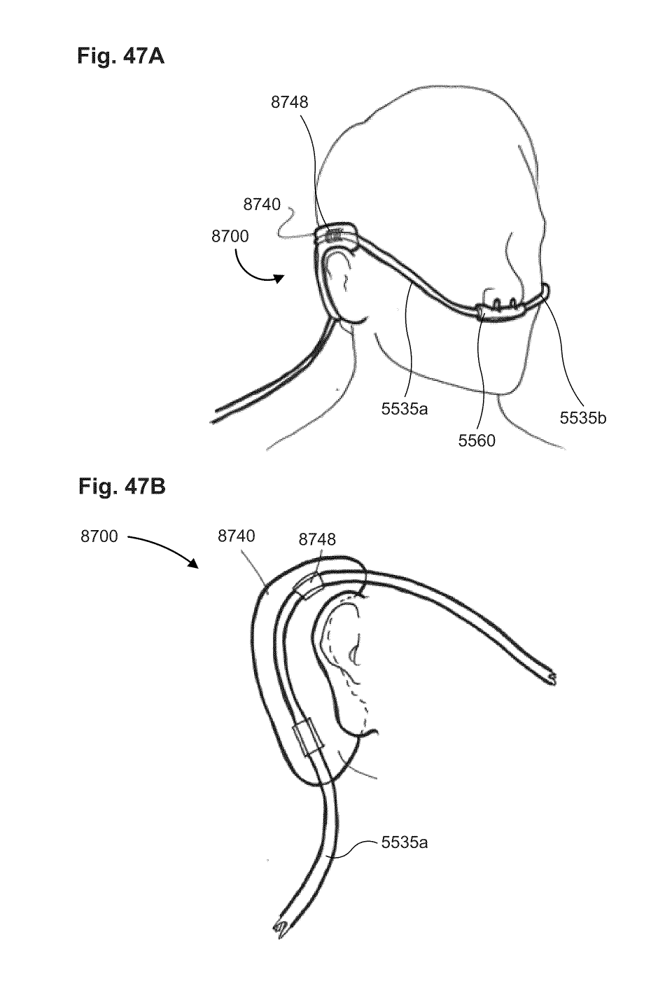

In some embodiments, a mounting assembly can include ear mounts configured to be work behind the ears of a user. For example, as shown in FIGS. 47A and 47B, a mounting assembly 8700 for mounting the face piece 5560, as described before herein, includes a set of ear mounts 8740. The mounting assembly 8700 can be used with any of the nasal cannula assemblies described herein, for example the nasal cannula assembly 500. The ear mounts 8740 are configured to be worn behind the ear of the user. In some embodiments, the ear mounts 8740 can be configured to adhere behind the ear of loop (e.g., with an adhesive) around a portion of the ear such that ear mounts 8740 can be disposed behind the ears of the user. In other embodiments, the ear mounts can have hooks, loops, or any other features that can loop around the ear, thereby allowing the ear mounts to be removably disposed on the ears of the user. The ear mounts 8740 can be formed from a rigid material, for example, a plastic. In some embodiments, the ear mounts 8740 can be formed from or covered with a soft padded material. A set of securing bands 8748 are disposed on a side wall of the ear mounts 8740 which are configured to secure a first face piece tube 5535a and a second face piece tube 5535b. The securing bands 8748 can be for example, elastic loops, or clamps. The face piece tubes are coupled to the face piece 5560. The ear mounts 8740 can therefore be worn behind the ears the user such that the first piece tube 5535a and the second face piece tube 5535b loop around the ears of the user and the face piece 5560 is disposed in proximity of nares of the user. In some embodiments, the ear mounts can include hearing aids configured to be worn on an ear of the patient that have been modified to include securing bands for mounting the face piece tubes.

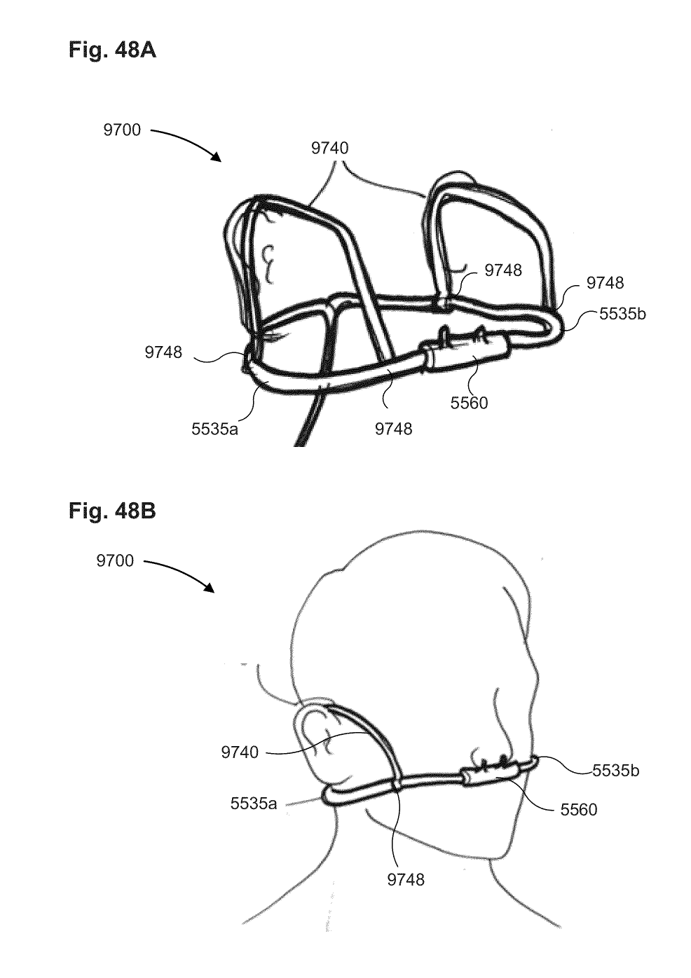

In some embodiments, a mounting assembly can include support straps that loop around the ears of a user. Referring now to FIGS. 48A-B, a mounting assembly 9700 includes a set of support straps 9740 coupled to a first face piece tube 5535a and a second face piece tube 5535b. The mounting assembly 9700 can be used with any of the nasal cannula assemblies described herein, for example the nasal cannula assembly 500. The support straps 9740 can be formed from a fabric, a plastic, or metal. In some embodiments, the support straps 9740 can be a hollow or solid tube. In some embodiments, the support straps can be rigid. In other embodiments, the support straps 9740 can be flexible. Each of the set of support straps 9740 includes a securing mechanism 9748 at each end of the support strap 9740. The securing mechanism 9748 can include, for example, an elastic loop, elastic band, clamp, hook, or any other suitable securing mechanism. The securing mechanism 9748 is configured to be coupled the set of support straps 9740 to the first face piece tube 5535a and the second face piece tube 5535b, such that the support straps 9740 can loop around the ears of the user. In this manner, a face piece 5560, as described before herein, which is coupled to the first face piece tube 5535a and the second face piece tube 5535b can be mounted in close proximity to the nares of the user.

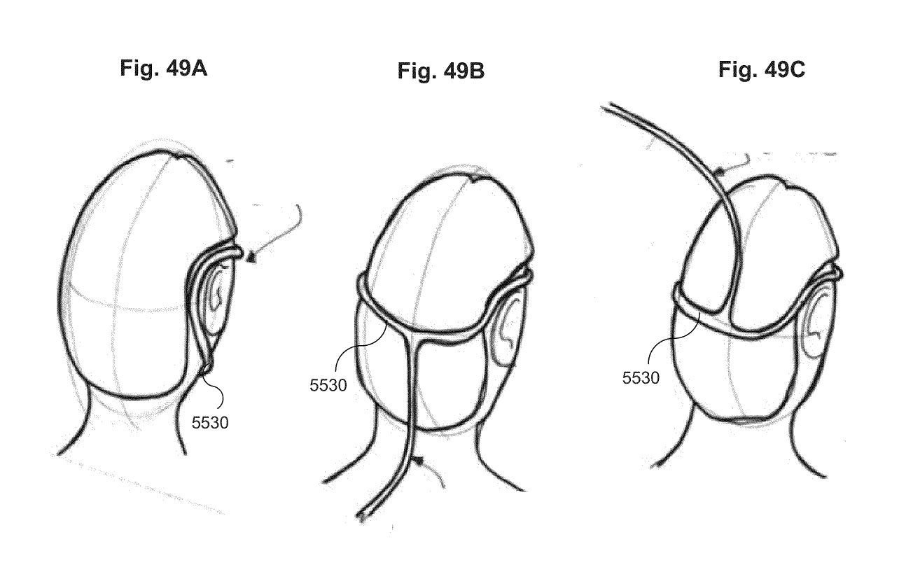

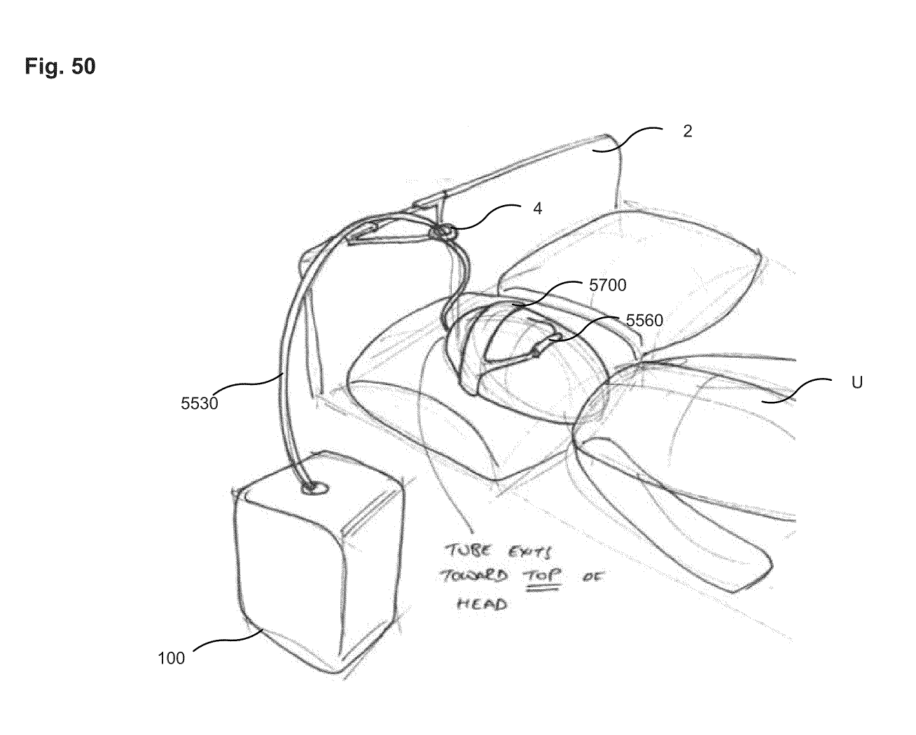

The supply line and/or a face piece tube can be disposed in any suitable manner around the face and/or head of a user such that a face piece, for example the face piece 5560 is securely mounted on the face of the user and the supply line and/or face piece tube does not obstruct the normal activity of the user. For example, as shown in FIG. 49A-C, in a first configuration, a supply line 5530, which can be substantially similar to the supply line 530 or any other supply line described herein, can be looped around the ears of the user and under the chin of the user (FIG. 49A). In a second configuration, the supply line 5530 can be looped around the ears of the user and run down the back of the user (FIG. 49B). In a third configuration, the supply line can be configured to be looped around the ears of the user and is then pulled over the head of the user. The third configuration can be particularly suitable when the user is lying flat on bed. For example, referring now to FIG. 50, a user U can have the mounting assembly 5700, or any other mounting assembly described herein, mounted on the head of the user U. The face piece 5560 (or any other face piece described herein) mounted on mounting assembly 5700 is disposed in proximity of the nares of the user U. The supply line 5530 is in fluidic communication with the face piece 5530 and at least a portion of the supply line 5530 is coupled to the mounting assembly 5700. The supply line 5530 is configured to receive an aerosol flow from the aerosol preparation assembly 100 and deliver the aerosol flow to the face piece 5560. The user U can lie flat on the user's U back on a bed 2. A hook 4 (e.g., a loop or a ring) is disposed on the bed 2. The supply line 5530 can be disposed in the third configuration, (i.e., pulled over the head of the user U) such that at least a portion of the supply line 5530 passes through the hook 4. In this way, the supply line 5530 can be supported by the hook 4, such that any movement of the user U on the bed 2 does not effect the configuration of the supply line 5530 which remains disposed over the head of the user U.

The following examples show face pieces that can be included in a cannula assembly and configured to deliver an aerosol flow to a user such that rainout and/or sputter are reduced. These examples are for illustrative purposes only and are not meant to limit in any way the scope of the present disclosure.

### EXAMPLES

### Effect of Bilateral vs. Unilateral Flow on Rainout

Commercial nasal cannula assembly components were used to produce three customized cannula assemblies, the custom #1 cannula, the custom #2 cannula, and the custom #3 cannula. The custom #1 cannula was formed by coupling a 1600HF™ face piece available from Salter labs with two supply lines that were included in a Salter labs 1650™ cannula assembly. The 1600HF™ face piece is a bilateral flow face piece, that is it includes an inlet to receive opposing aerosol flow each side of the face piece. The 1600HF™ included a pair of curved delivery protrusions which had an internal diameter of about 3.0 mm. The 1650 supply lines were about 7 foot long and have a smooth bore.

The custom #2 cannula was formed by cutting out the curved delivery protrusions from the 1600HF™ face piece. Each of the delivery protrusion was fused with a 1650 supply line to realize the custom #2 cannula assembly. In this manner, the custom #2 cannula assembly is configured to deliver a bilateral flow through each delivery protrusion, separately via each of the supply lines.

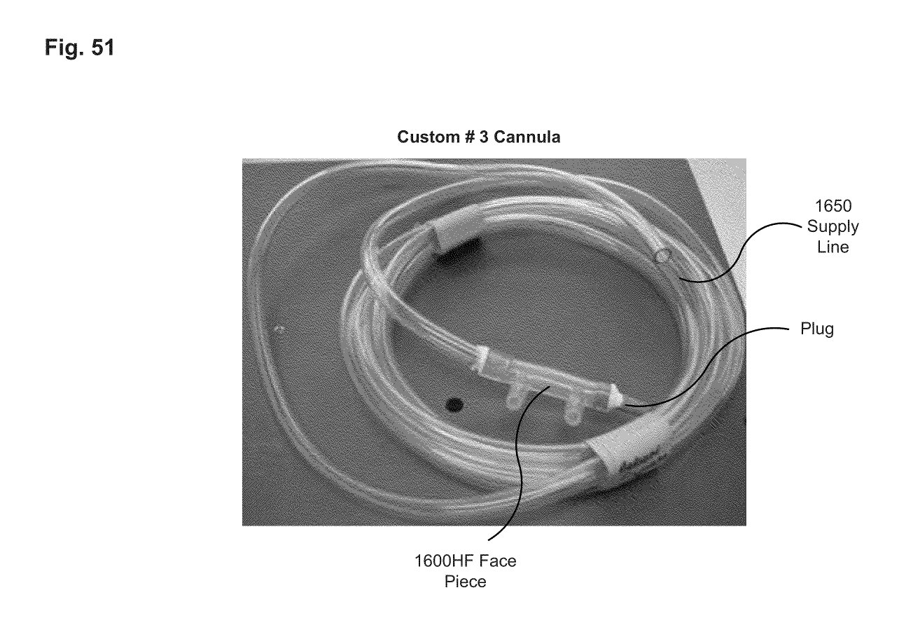

The custom #3 cannula was formed by blocking one inlet of a 1600HF™ face piece with a plug and coupling a 1650 supply line to the other open inlet of the face piece. In this manner, the custom #3 cannula was configured have a unilateral flow, i.e., a flow through only one inlet which is divided into a first portion delivered by the first delivery protrusion and a second portion delivered by the second delivery protrusion. FIG. 51 shows the custom #3 cannula. Such a unilateral configuration is similar to the configuration of many of the face pieces shown and described herein, such as the face pieces 5560 shown and described above.

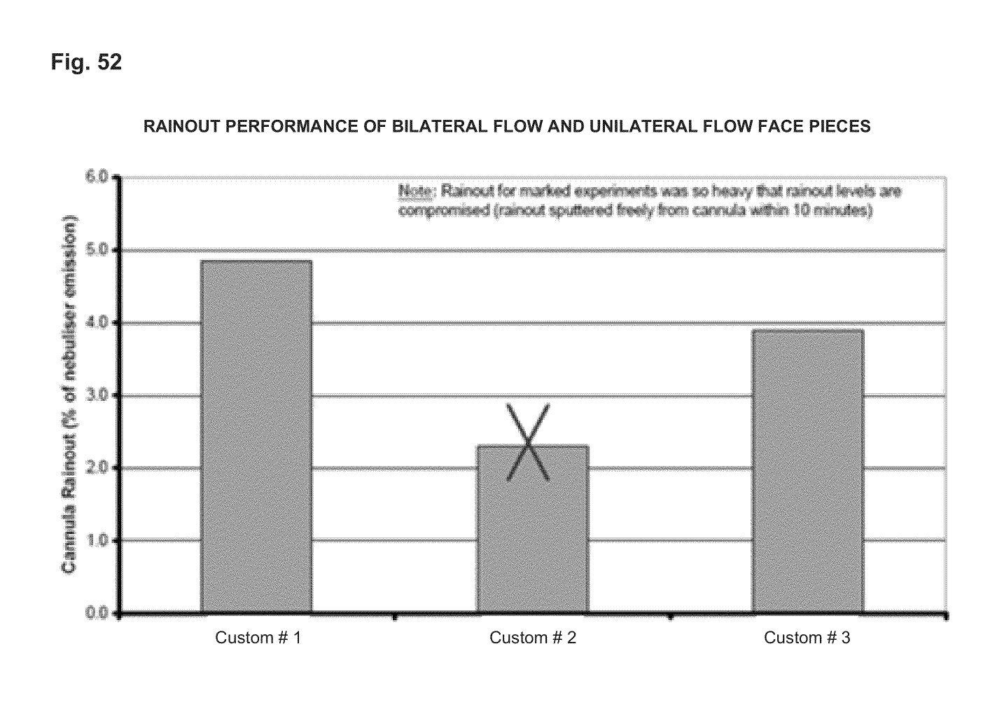

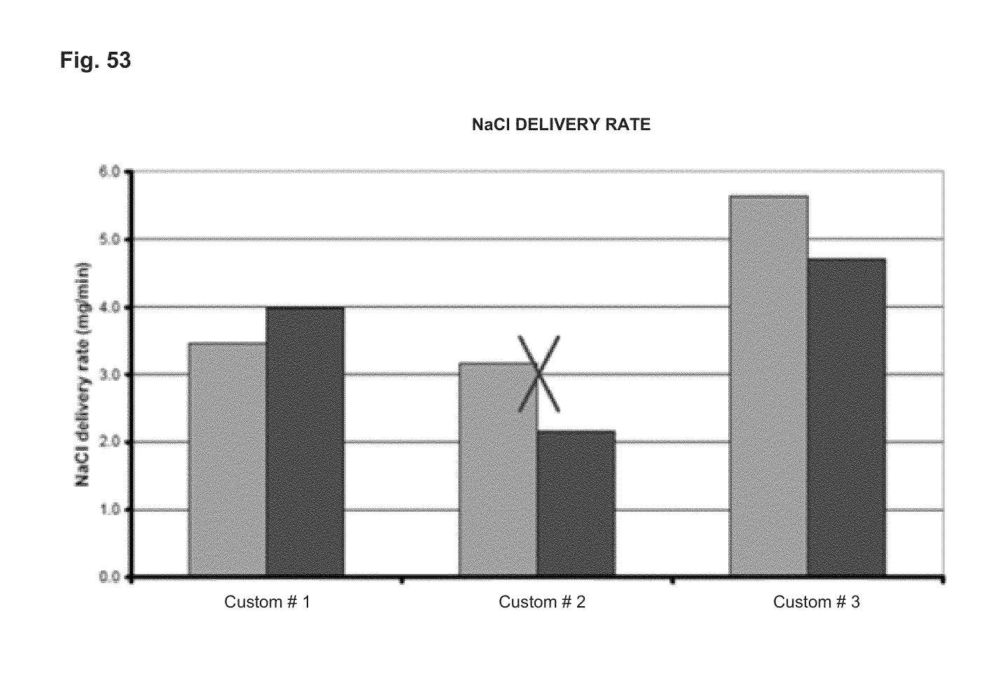

Each of the custom #1, the custom #2, and the custom #3 cannula were tested for rainout performance. An Aeroneb Pro™ Nebuliser (#104002-007) was used to deliver a 2 LPM aerosol flow to each of the cannulas. The aerosol included a 7% hypertonic saline (HS) solution. The aerosol flow was maintained for 30 minutes through each of the cannulas and the cannula rainout as a percentage of nebulizer emissions was measured. Two runs were performed on each cannula assembly and the rainout results were averaged. FIG. 52 shows the average rainout data from each of the cannulas while FIG. 53 shows the mass of NaCl delivered by each cannula for each run. As shown in FIG. 52 and FIG. 53, the custom #1 cannula demonstrated an average rainout of about 4.8% and an average NaCl mass delivered of about 3.7 mg/ml. The custom #2 cannula became occluded very quickly and provided poor performance. Thus, the rainout data indicated in FIG. 52 and the NaCl delivery data in FIG. 53 for the custom #2 cannula was compromised, and is not a reliable indicator of the actual rainout generated by this bilateral flow cannula. In contrast, the unilateral flow custom #3 cannula had an average rainout of about 3.8%, which is lower than that for the bilateral custom #1 cannula. The average delivered mass of NaCl for the unilateral flow custom #3 cannula was about 5.0 mg/ml, which is higher than that for the custom #1 cannula. This data shows that unilateral aerosol flow can reduce rainout while maintaining or even improving the delivery of the aerosolized medicament.

### Rainout Performance of Face Pieces Having Circular and Non-Circular Cross-Sections

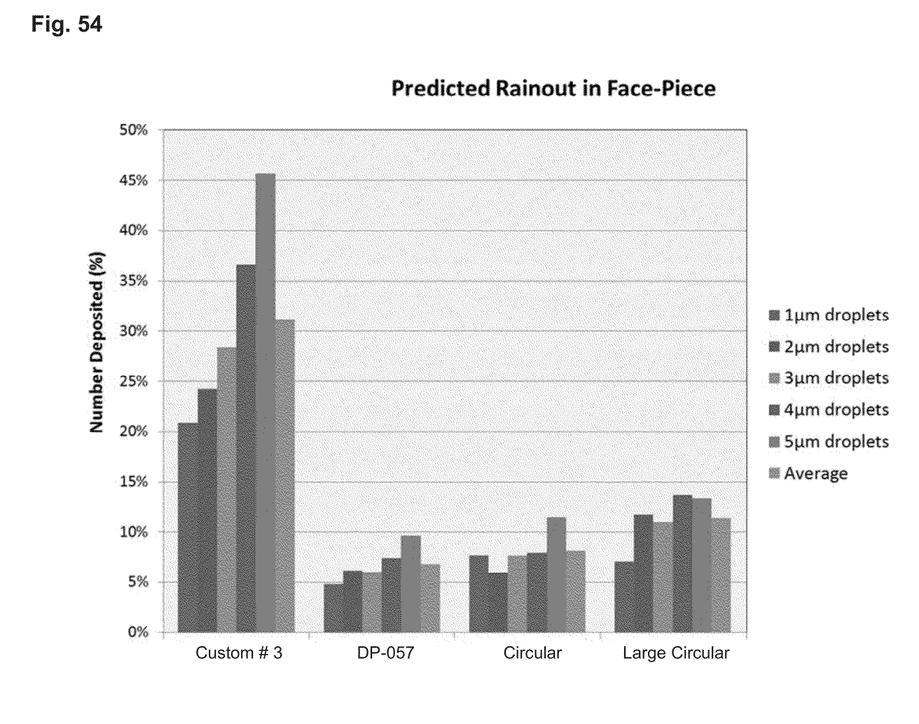

FIG. 54 shows the simulated rainout performance of face pieces having various cross-sectional shapes (e.g., to compare circular and non-circular cross-sections). The rainout simulations were performed using a computational fluid dynamics model in FLUENT™ fluid modeling software. Droplets of various size and mass were modeled and were released at the inlet of the simulated face pieces. The trajectories of the droplets were calculated and it was determined whether the droplets were delivered by the face piece through the nasal prongs, or were retained in the face piece (i.e., rainout).

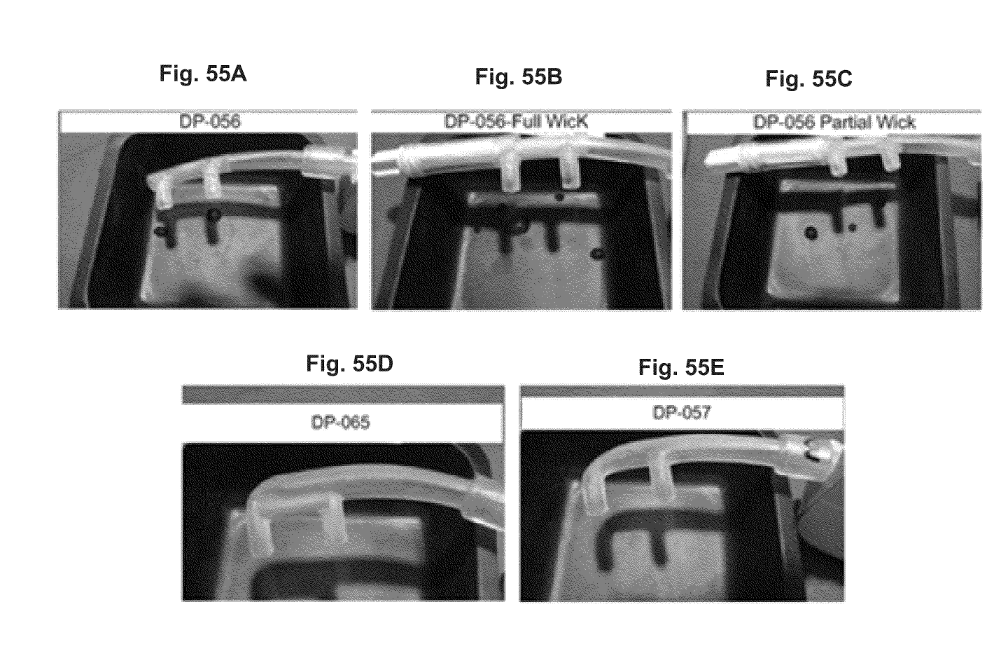

The DP-057 face piece, which is shown below in FIG. 55E, is substantially similar to the face piece 5560 described with respect to FIGS. 6-14. In particular, the DP-057 face piece includes a plenum portion having an elliptical and/or oblong cross-section, and includes an end side wall that defined a curved surface as shown in FIGS. 6-14. The DP-057 also included a flow restriction as described with respect to the face piece 5560. The DP-057 face piece differs in that it does not include the second end portion shown in FIGS. 6-14, which is used solely for mounting, and does not impact the flow performance. The DP-057 face piece was formed using stereolithography.

The circular face piece modeled FIG. 54 was similar to the DP-057 face piece, except that the cross-section of the plenum portion was substantially circular and had a diameter of about 5.6 mm. The large circular face piece was substantially similar to the circular face piece, except that the diameter of the plenum portion of the large circular face piece was about 8.3 mm, about two times greater than that for the circular face piece.

As shown in FIG. 54, the DP-057, the predicted rain out performance for the circular and the large circular face pieces demonstrated a much lower rainout percentage for a variety of aerosol droplet sizes, as compared to the bilateral Custom #3 face piece. On average about 7% of the 1-5 μm droplets were deposited in the DP-057 face piece (having a non-circular cross-section) as compared to about 8% of the 1-5 μm droplets deposited in the circular face piece and about 10% μm droplets deposited in the large circular face piece. These were substantially lower than the rainout performance of the Custom #3 face piece which had about 31% of the droplets deposited. Furthermore, the DP-057 face piece which has the elliptical cross-section demonstrated less rainout than the circular face piece and the large circular face piece. This shows that the change in cross-sectional shape from a circular shape of the first end portion to a non-circular shape (e.g., elliptical) of the plenum portion can reduce rainout.

### Rainout Performance of Various Unilateral Flow Face Pieces

FIG. 55A-E shows various unilateral flow face pieces that were tested for their rainout performance. FIG. 55A shows the DP-056 face piece which is based on the custom #3 cannula face piece described before. The DP-056 face piece was stereolithographically formed and included an inlet portion in fluidic communication with a plenum portion. The plenum portion had a circular cross-section and an end side wall with a flat (i.e., non-curved) surface orthogonal to the flow path of the plenum portion.

FIG. 55B shows the DP-056 Full Wick face piece which was based on the DP-056 face piece. The flat end side wall of a DP-056 face piece was cut open and a wick holding tube was coupled to the cut open end. A full paper wick (i.e., a paper wick disposed on the entire circumference of the inner surface of the wick holding tube) was disposed in the wick holding tube and the wick holding tube was blocked with a plug such that DP-056 Full Wick defined a unilateral flow path for an aerosol flow.

The DP-056 Partial Wick face piece shown in FIG. 55C was the same as the DP-056 Full Wick face piece other than the paper wick was disposed only on half the circumference of the wick holding tube.

The DP-065 face piece shown in FIG. 55D was substantially similar to the face piece 8560 described with respect to FIG. 18 and includes a late bifurcation. The DP-065 face piece was formed using stereolithography. The DP-065 face piece included a plenum portion that had an elliptical cross-section and included an end side wall that defined a curved surface.

The DP-057 face piece shown in FIG. 55E was substantially similar to the face piece 5560 described with respect to FIGS. 6-14, but did not include the second end portion. The DP-057 face piece includes a plenum portion that had an elliptical cross-section and included an end side wall that defined a curved surface. The DP-057 also included a flow restriction as described with respect to the face piece 5560. The DP-057 face piece was formed using stereolithography.

Each of the unilateral flow face pieces included a pair of delivery protrusions that had a curved center line and an inner diameter of about 3.0 mm. Each of the unilateral flow face pieces was coupled to a 1650 supply line. An Aeroneb Pro™ Nebuliser (#104002-007) was used to deliver a 600 mg/min (18 ml/30 min) of aerosol flow which included a 7% hypertonic saline (HS) solution for a period of 30 minutes. Three runs were performed on each face piece. The rainout and sputter, NaCl delivered through the delivery protrusions and the size of the droplet was measured for each run and averaged.

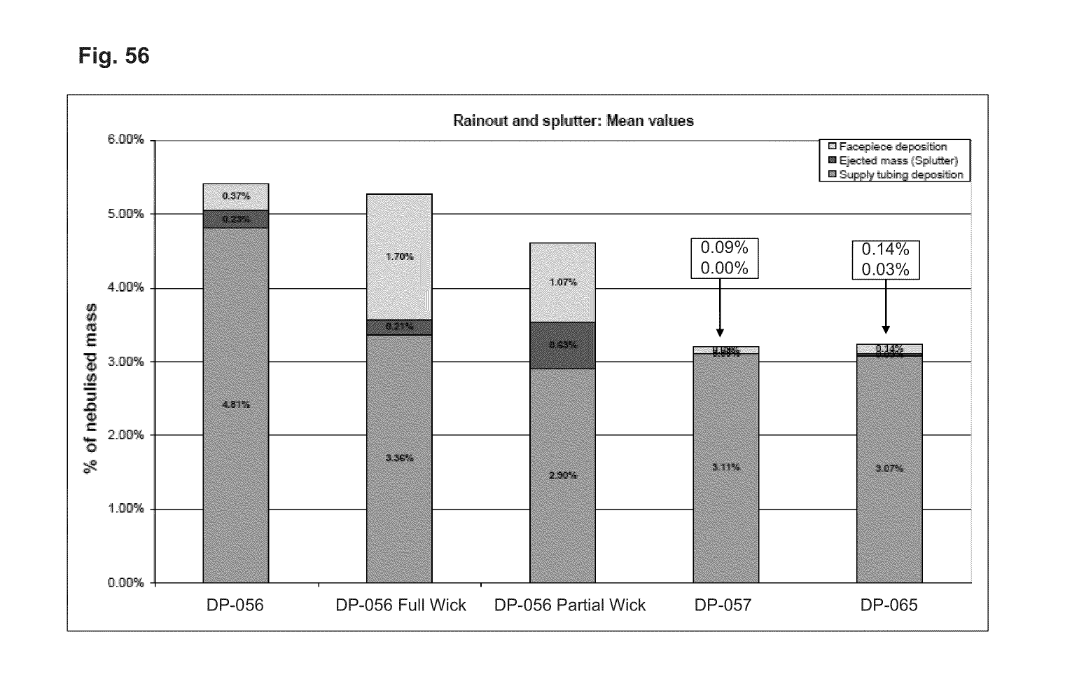

As shown FIG. 56, rainout (identified as “face piece deposition” in the plot) was observed in the DP-056 face piece, DP-056 Full Wick face piece, and DP-056 Partial Wick face piece, while negligible rainout was observed for the DP-057 and the DP-065 face pieces after 30 minutes of operation. As shown in the bar graph of FIG. 56, the DP-056 face piece, DP-056 Full Wick face piece, and DP-056 Partial Wick face piece had substantial amount of rainout and sputter (sputter is identified as the “ejected mass” in the plot). In comparison, DP-065 face piece had negligible amounts of sputter and rainout, while the DP-057 face piece showed negligible amounts of rainout and almost no sputter. As shown in FIG. 56, each of the cannula assemblies were susceptible to some amount of rainout collected in the supply tubing.

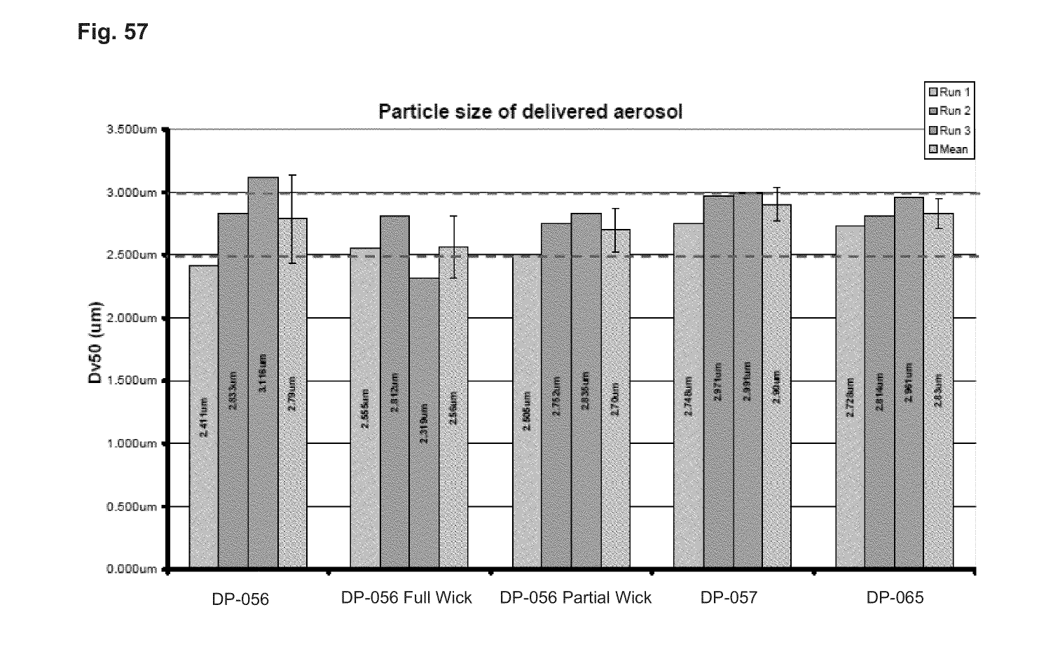

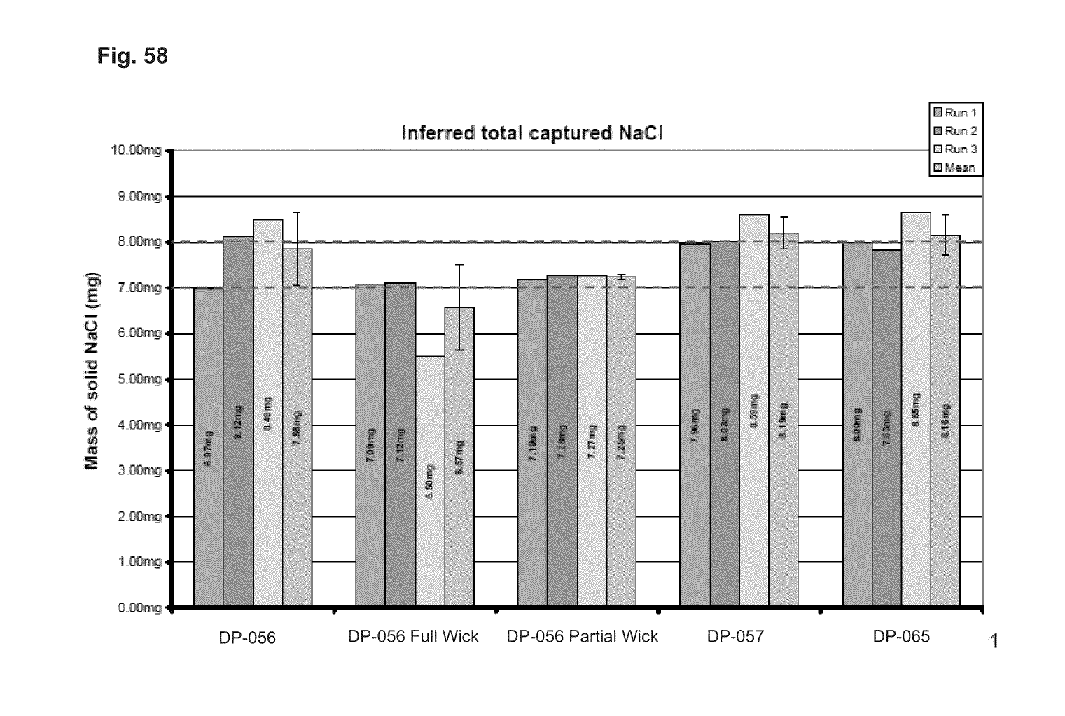

FIG. 57 shows the particle size of the delivered aerosol and FIG. 58 shows the mass of NaCl delivered by each unilateral flow cannula after 30 minutes of operation. All face pieces demonstrated similar performance of particle size of aerosol delivered (Dv50) in the range of about 2.5 mm to about 3.0 mm, and a total delivered NaCl in the range of about 7.0 mg to about 8.0 mg, although the DP-056 Full Wick and the DP-056 Partial Wick face pieces performed on the lower end of the range. These data show that the unilateral flow face pieces DP-057 and DP-065 that include a curved end wall, an elliptical cross-section and a flow restriction can deliver the desired amount of an aerosol flow having a particle size in the desired range (e.g., about 2.5 mm to about 3.0 mm) while significantly reducing the rainout and sputter from the face piece.

### Balanced Flow in the Delivery Protrusions by Flow Restriction

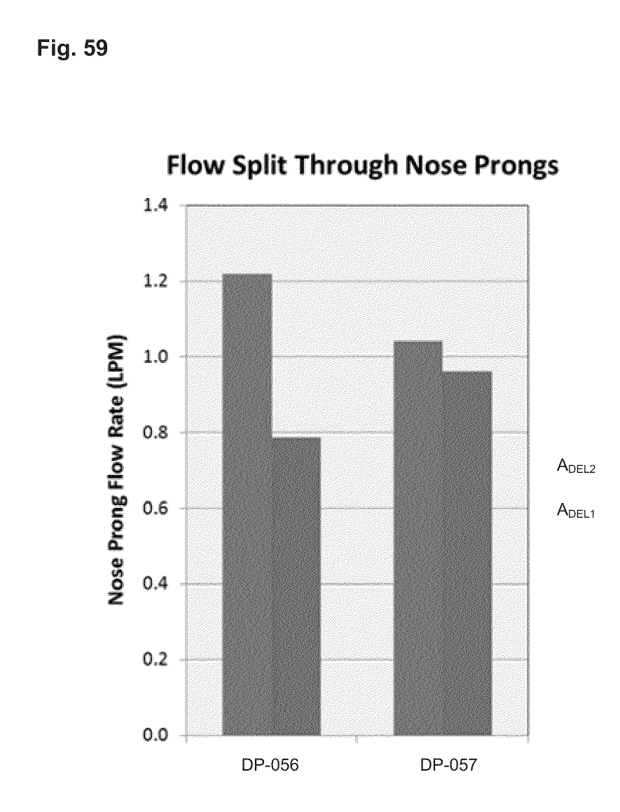

Computational fluid dynamic (CFD) simulations were performed using FLUENT™ fluid modeling software, on computer generated models of the DP-056 face piece and the DP-057 face piece to determine the impact of the flow-restriction on a first portion of the aerosol delivered to the first delivery protrusion A_{DEL1} and a second portion of the aerosol delivered to the second delivery protrusion A_{DEL2}. Each model was configured to simulate an aerosol at a flow rate of 2 LPM. As shown in FIG. 59, the DP-056 demonstrated a greater imbalance in the flow between the two delivery protrusions, with A_{DEL1} being about 1.22 LPM and A_{DEL2} being about 0.78 LPM. In contrast the DP-057 face piece that includes the flow restriction had a substantially balanced flow with A_{DEL1} about 1.08 LPM and A_{DEL2} about 0.92 LPM.

Curved vs. Straight Delivery Protrusions

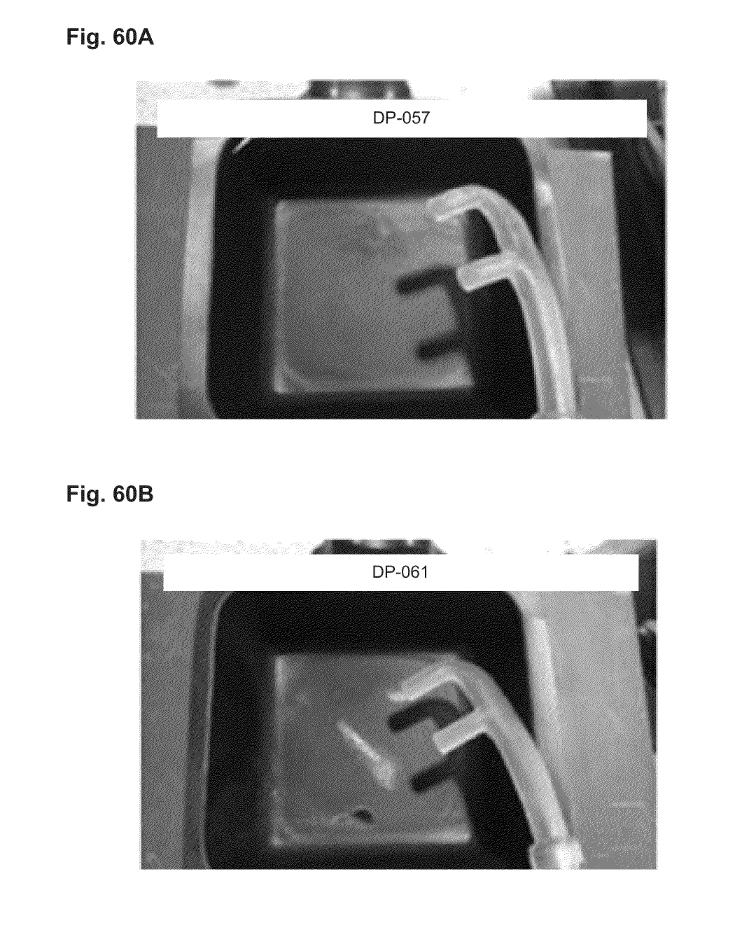

Experiments were performed to observe the difference in rainout of curved delivery protrusions when compared to straight delivery protrusions. A unilateral flow face piece DP-061 was formed by stereolithography. The DP-061 was substantially similar to the DP-057 face except that the DP-061 face piece included a first delivery protrusion and a second delivery protrusion defining a straight aerosol flow path. An Aeroneb Pro™ Nebuliser (#104002-007) was used to deliver a nominal aerosol flow of about 550 mg/min, which included a 7% hypertonic saline (HS) solution for a period of 30 minutes. Two runs were performed on each face piece. The rainout and sputter was measured for each run. Photographs showing the rainout collected for one of the runs for each face piece are shown in FIG. 60A (for the DP-057 design) and FIG. 60B (for the DP-061 design). The DP-056 demonstrated a rainout of about 0.0% to about 0.1% of the nebulized mass while the DP-061 demonstrated a rainout of about 0.0% to about 0.4% of the aerosolized mass. Furthermore, the DP-057 face piece demonstrated a sputter of about 0.2% of the aerosolized mass, and the DP-061 face piece demonstrated a sputter of about 0.1%. While the rainout and sputter demonstrated by the curved and the straight protrusions is substantially the same, the DP-057 anecdotally delivered a more repeatable performance. Additionally, the curved protrusions can be ergonomically preferable.

### Performance Over Extended Periods of Operation

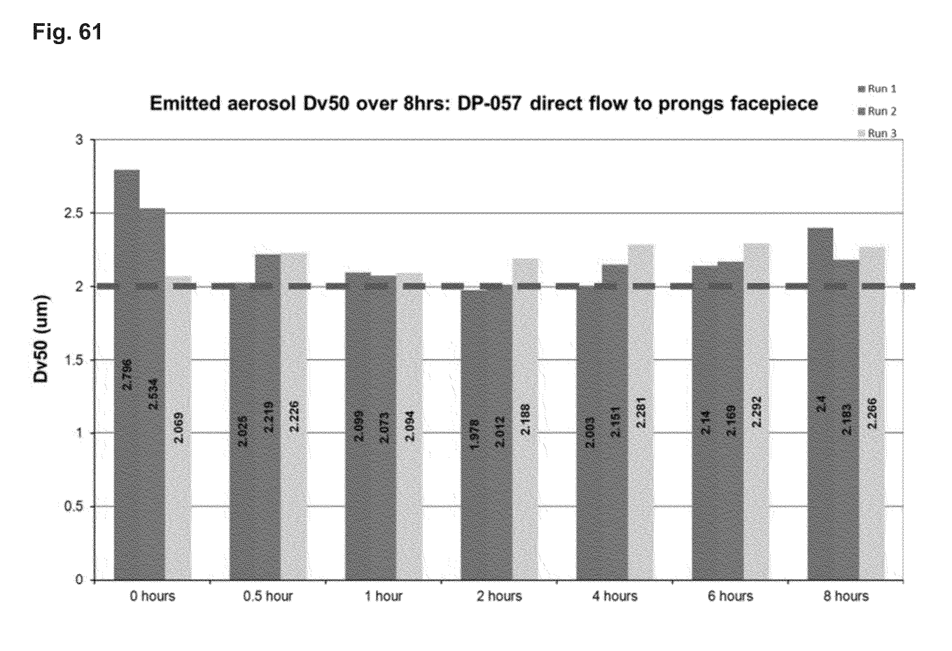

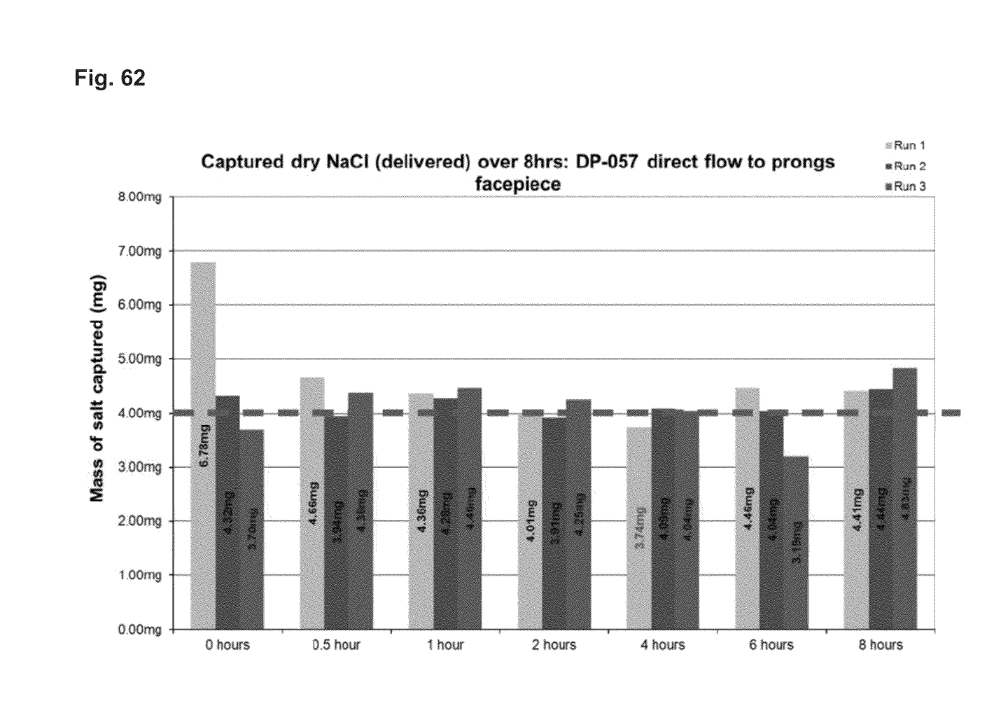

The performance of the DP-057 face piece was tested for over 8 hours of operation, which represents a desired operation time for methods using the face pieces described herein. An Aeroneb Pro™ Nebuliser (#104002-007) was used to deliver a nominal aerosol flow of about 550 mg/min, which included a 7% hypertonic saline (HS) solution for a period of 8 hours. FIG. 61 shows the size (Dv50) of the droplets of the delivered aerosol from the DP-057 face piece, measured at period time intervals. The average droplet size of the delivered aerosol remains substantially constant over 8 hours of operation with a Dv50 of about 2 μm. Similarly, as shown in FIG. 62, the delivery rate of NaCl from the delivery protrusions of the DP-057 face piece was substantially similar at about 4 mg of NaCl/min. These data demonstrate the repeatable performance of the unilateral face piece with the curved surface and non-circular cross-sectional shape over long periods of time.

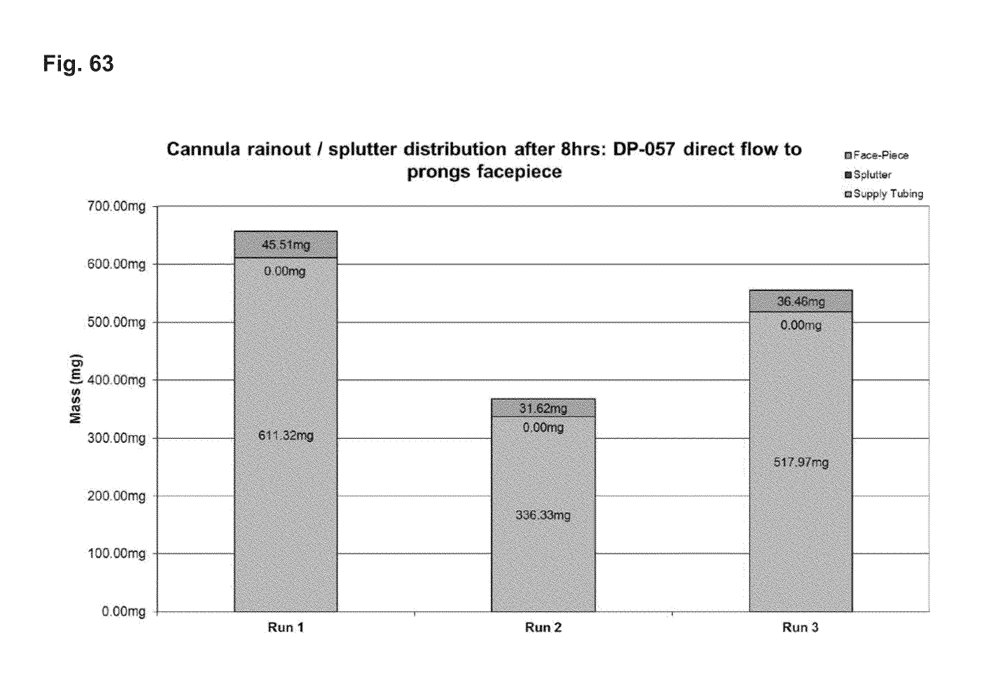

FIG. 63 shows the sputter performance of the DP-057 face piece. Three runs were performed and the amount of rainout in the DP-057 face piece and the supply tubing coupled thereto, as well as the sputter from DP-057 face piece was measured. Over 8 hours of operation no sputter was emitted from the delivery protrusions (this is indicated by the notation of “0.00 mg” on the bar, and the absence of a dark bar representing the “sputter” as indicated in the legend in FIG. 63). As indicated there was a minimal amount of rainout that collected within the face piece (on the order of 30-40 mg).

### Effect of Manufacturing Process on Rainout

These experiments were designed to highlight the impact of the manufacturing process on the rainout characteristics of a face piece. A face piece DP-056-M was formed using a molding process. The DP-056-M face piece was substantially similar in size, shape, structure, and material to the DP-056 face piece described herein, other than it was formed using a molding process (as opposed to a stereolithography process). Two different nebulizers were used to deliver an outlet aerosol containing about 7% hypertonic saline solution at a flow rate of about 2 LPM to each of the DP-056-M and the DP-056 face piece, for about 30 minutes, and the sputter emitted from the nasal prongs of each of the face piece was measured. After about thirty minutes of operation, the sputter emitted from the DP-056 face piece was about 0.24% and about 1.51% of the nebulized mass, for each run, respectively. In contrast, after thirty minutes of operation, the sputter emitted from the DP-056-M face piece was about 3.36% and about 10.65%, for each run, respectively. This indicates that with all other conditions being same, the manufacturing process can impact the rainout and/or sputter of the face piece. The molding process seems to produce a face piece which has a surface energy and/or other characteristics that are more susceptible to producing rainout and sputter relative to the SLA process. The surface properties of the DP-056-M face piece, or any other face piece described herein can potentially be improved to reduce rainout and/or sputtering by performing the molding process without the use of a release agent that can get coated on an inner surface of the face piece. Furthermore, post-processing as described before herein can also be performed.

### Active Agents and Therapies Devliverable by Nasal Cannula Assemblies

Suitable therapeutic agents that can be administered by embodiments of the nasal cannula assembly described herein, and diseases and disorders treatable with these agents are listed below:
Exemplary agents targeting pulmonary tissue are listed below in “Drug Classes Suitable for Targeting Pulmonary Tissues.”
Exemplary agents targeting extra-pulmonary tissue are listed below in “Drug Classes Suitable for Targeting Extra-pulmonary Tissues.” Only such agents that can be formulated to reach therapeutically effective levels in the extra-pulmonary tissues of interest are suitable.
All other classes of therapeutic agents, suitable for use either in pulmonary or extra-pulmonary space, are listed below in “All Other Drug Classes Suitable for Local or Systemic Administration.”
Diseases and conditions that can be treated by therapeutic agents administered via the embodiments of the nasal cannula assembly described herein by either locally acting (i.e. in the lung and nasal passages) or systemically acting (i.e. all extra-pulmonary compartments) is provided in “List of Diseases and Conditions Treated by Therapies Administered by Nasal Cannula Assemblies.”

### Drug Classes Suitable for Targeting Pulmonary Tissue

These agents include but are not limited to agents that (i) enhance or facilitate mucus clearance; (ii) have antimicrobial activity; (iii) have anti-inflammatory activity; (iv) or have bronchodilator activity; and (v) all other agents currently administered by inhalation via nebulizers, MDIs and DPIs. For agents with undesirable safety or tolerability properties due to high local or systemic concentration following bolus administration via nebulizer, administration by inhalation over the course of 8 to 24 hours or overnight to a patient via nasal cannula may improve the therapeutic index for such agents.

Exemplary Agents that Facilitate Mucus Clearance

Adequate mucus clearance (MC) is a crucial factor in the maintenance of normal airway health, is dependent on mucus rheology, airway hydration, and ciliary beat frequency (CBF). Abnormal mucus clearance is an important contributor to the phenotype of patients with chronic bronchitis due to environmental or genetic causes. Normal mucus clearance requires 1) adequate hydration of the airway surface and 2) an absence of strong adhesive interaction between the mucus and cell surface. Hydration is formally defined by the concentrations of mucins in the periciliary and mucus layers. Ion transport properties regulate the amount of salt and water (i.e. the solvent) and goblet cells and glands control the concentration of mucins on the airway surface. Both cystic fibrosis (CF) patients and subjects with chronic bronchitis associated with cigarette smoke exposure, i.e., COPD (Chronic Obstructive Pulmonary Disease), exhibit increases in mucus concentration as quantified by % solids, as a result of reduced airway hydration and mucin hypersecretion, consequent to goblet cell and glandular hyperplasia. Both as a function of disease severity, and in acute exacerbations, raised mucin/mucus concentrations produce adherent mucus that sticks to epithelial cells, initiates inflammatory responses and airway wall injury, and serves as a growth medium for pathogenic microorganisms (Boucher, R. C., “New concepts of the pathogenesis of cystic fibrosis lung disease”, European Respiratory Journal 23(1):146-158 (2004) and Matsui, H., Grubb, B. R., Tarran, R., Randell, S. H., Gatzy, J. T., Davis, C. W., and Boucher, R. C. “Evidence for periciliary liquid layer depletion, not abnormal ion composition, in the pathogenesis of cystic fibrosis airways disease”, Cell 95:1005-1015 (1998) and Matsui, H., Wagner, V. E., Hill, D. B., Schwab, U. E., Rogers, T. D., Button, B., Taylor, R. M., 2nd, Superfine, R., Rubinstein, M., Iglewski, B. H., et al., “A physical linkage between cystic fibrosis airway surface dehydration and Pseudomonas aeruginosa biofilms,”, Proc. Natl. Acad. Sci. USA 103:18131-18136 (2006)).

Osmolytes

Active compounds may be ionic osmolytes (i.e., salts), or may be non-ionic osmolytes (i.e., sugars, sugar alcohols, and organic osmolytes). It is to be noted that all racemates, enantiomers, diastereomers, tautomers, polymorphs and pseudopolymorphs and mixtures of the osmotically active compounds are suitable for use with disclosed embodiments.

Active osmolytes useful in the disclosed embodiments that are ionic osmolytes include any salt of a pharmaceutically acceptable anion and a pharmaceutically acceptable cation. Preferably, either (or both) of the anion and cation are non-absorbable (i.e., osmotically active and not subject to rapid active transport) in relation to the airway surfaces to which they are administered. Such compounds include but are not limited to anions and cations that are contained in FDA approved commercially marketed salts, see, e.g., Remington: The Science and Practice of Pharmacy, Vol. II, pg. 1457 (19.sup.th Ed. 1995), incorporated herein by reference, and can be used in any combination including their conventional combinations.

Pharmaceutically acceptable osmotically active anions that can be used to implement the disclosed embodiments include, but are not limited to, acetate, benzenesulfonate, benzoate, bicarbonate, bitartrate, bromide, edetate, camsylate (camphorsulfonate), carbonate, chloride, citrate, edisylate (1,2-ethanedisulfonate), estolate (lauryl sulfate), esylate (1,2-ethanedisulfonate), fumarate, gluceptate, gluconate, glutamate, glycollylarsanilate (p-glycollamidophenylarsonate), hexylresorcinate, hydrabamine (N,N′-di(dehydroabietyl)ethylenediamine), hydroxynaphthoate, iodide, isethionate, lactate, lactobionate, malate, maleate, mandelate, mesylate, methylnitrate, methylsulfate, mucate, napsylate, nitrate, nitrite, pamoate (embonate), pantothenate, phosphate or diphosphate, polygalacturonate, salicylate, stearate, subacetate, succinate, sulfate, tannate, tartrate, teoclate (8-chlorotheophyllinate), triethiodide, bicarbonate, etc. Particularly preferred anions include chloride, sulfate, nitrate, gluconate, iodide, bicarbonate, bromide, and phosphate.

Pharmaceutically acceptable cations that can be used to implement the disclosed embodiments include, but are not limited to, organic cations such as benzathine (N,N′-dibenzylethylenediamine), chloroprocaine, choline, diethanolamine, ethylenediamine, meglumine (N-methyl D-glucamine), procaine, D-lysine, L-lysine, D-arginine, L-arginine, triethylammonium, N-methyl D-glycerol, and the like. Particularly preferred organic cations are 3-carbon, 4-carbon, 5-carbon and 6-carbon organic cations. Metallic cations useful in the practice of the disclosed embodiments include but are not limited to aluminum, calcium, lithium, magnesium, potassium, sodium, zinc, iron, ammonium, and the like. Particularly preferred cations include sodium, potassium, choline, lithium, meglumine, D-lysine, ammonium, magnesium, and calcium.

Specific examples of osmotically active salts that may be used with the sodium channel blockers described herein to carry out the disclosed embodiments include, but are not limited to, sodium chloride, potassium chloride, choline chloride, choline iodide, lithium chloride, meglumine chloride, L-lysine chloride, D-lysine chloride, (usually seen as the HCl salt) ammonium chloride, potassium sulfate, potassium nitrate, potassium gluconate, potassium iodide, ferric chloride, ferrous chloride, potassium bromide, etc. Either a single salt or a combination of different osmotically active salts may be used to carry out the disclosed embodiments. Combinations of different salts are preferred. When different salts are used, one of the anion or cation may be the same among the differing salts.

Osmotically active compounds of the disclosed embodiments also include non-ionic osmolytes such as sugars, sugar-alcohols, and organic osmolytes. Sugars and sugar-alcohols useful in the practice of the disclosed embodiments include but are not limited to 3-carbon sugars (e.g., glycerol, dihydroxyacetone); 4-carbon sugars (e.g., both the D and L forms of erythrose, threose, and erythrulose); 5-carbon sugars (e.g., both the D and L forms of ribose, arabinose, xylose, lyxose, psicose, fructose, sorbose, and tagatose); 6-carbon sugars (e.g., both the D and L forms of altose, allose, glucose, mannose, gulose, idose, galactose, and talose), and the 7-carbon sugars (e.g., both the D and L forms of allo-heptulose, allo-hepulose, gluco-heptulose, manno-heptulose, gulo-heptulose, ido-heptulose, galacto-heptulose, talo-heptulose). Additional sugars useful in the practice of the disclosed embodiments include raffinose, raffinose series oligosaccharides, and stachyose. Both the D and L forms of the reduced form of each sugar/sugar alcohol useful in the disclosed embodiments are also suitable active compounds. For example, glucose, when reduced, becomes sorbitol; within the scope of the invention, sorbitol and other reduced forms of sugar/sugar alcohols (e.g., mannitol, dulcitol, arabitol) are accordingly suitable active compounds.

Suitable osmotically active compounds additionally include the family of non-ionic osmolytes termed “organic osmolytes.” The term “organic osmolytes” is generally used to refer to molecules used to control intracellular osmolality in the kidney. See e.g., J. S. Handler et al., Comp. Biochem. Physiol., 117:301-306 (1997); M. Burg, Am. J. Physiol. 268: F983-F996 (1995), each incorporated herein by reference. Not intending to be bound by any particular theory, it appears that these organic osmolytes are useful in controlling extracellular volume on the airway/pulmonary surface. Organic osmolytes useful as active compounds for the disclosed embodiments include but are not limited to three major classes of compounds: polyols (polyhydric alcohols), methylamines, and amino acids. The polyol organic osmolytes considered useful in the practice of the disclosed embodiments include, but are not limited to, inositol, myo-inositol, and sorbitol. The methylamine organic osmolytes useful in the practice of disclosed embodiments include, but are not limited to, choline, betaine, carnitine (L-, D- and DL forms), phosphorylcholine, lyso-phosphorylcholine, glycerophosphorylcholine, creatine, and creatine phosphate. Suitable amino acid organic osmolytes include, but are not limited to, the D- and L-forms of glycine, alanine, glutamine, glutamate, aspartate, proline and taurine. Additional osmolytes useful in the practice of disclosed embodiments include tihulose and sarcosine. Mammalian organic osmolytes are preferred, with human organic osmolytes being most preferred. However, certain organic osmolytes are of bacterial, yeast, and marine animal origin, and these compounds are also useful active compounds for the practice of disclosed embodiments.

Under certain circumstances, an osmolyte precursor may be administered to the subject; accordingly, these compounds are also useful in the practice of the disclosed embodiments. The term “osmolyte precursor” as used herein refers to a compound which is converted into an osmolyte by a metabolic step, either catabolic or anabolic. Suitable osmolyte precursors of this invention include, but are not limited to, glucose, glucose polymers, glycerol, choline, phosphatidylcholine, lyso-phosphatidylcholine and inorganic phosphates, which are precursors of polyols and methylamines. Suitable precursors of amino acid osmolytes include proteins, peptides, and polyamino acids, which are hydrolyzed to yield osmolyte amino acids, and metabolic precursors which can be converted into osmolyte amino acids by a metabolic step such as transamination. For example, a precursor of the amino acid glutamine is poly-L-glutamine, and a precursor of glutamate is poly-L-glutamic acid.

In one embodiment, the osmolyte is hypotonic saline, isotonic saline, or hypertonic saline used as the active agent.

The Importance of Buffering Systems for Aerosolized Therapies

Buffering agents contained in pharmaceutical formulations are typically added to maintain the activity or stability of the pharmaceutical product. Furthermore, upon aerosolization and delivery of the aerosol to lung airway surfaces, the buffering agents can maintain the physiological pH of lung airway surfaces. For example, the pH of the airway surface liquid is regulated to values of ˜pH 7 to 7.4, and airways host defense in part depends on maintenance of the pH. Buffering agents used in formulations of aerosolized therapies, therefore, may be are useful for 1) preventing acidification of the airway surface occurring with hyperosmolar therapies; 2) normalization of the pH of the airway surface for diseases accompanied or associated or caused by lower than normal airway surface pH; and 3) to prevent or attenuate the drop in the airway surface following administration of aerosols that would otherwise be formulated at pH lower than the airway surface (pH=˜7 to 7.4) or causing acidification of the airway surface following deposition in the lung. The use of bicarbonate anion as a buffering agent may be particularly useful, given its natural role as a buffering agent on the airway surface, and its depletion in diseases associated or caused by CFTR dysfunction such as CF and COPD.

The buffering agent can be any compound comprising an anionic component which is able to maintain a pH from about 6.8 to about 7.6. In one embodiment, the anionic component is able to maintain a pH from about 6.9 to about 7.5. In another embodiment, the anionic component is able to maintain a pH from about 7.0 to about 7.4. Examples of the anionic component include, but are not limited to, carbonate (CO₃²⁻) and bicarbonate (HCO₃⁻). Examples of the buffering agent include, but are not limited to, any alkali metal and alkaline earth metal salt of carbonate and bicarbonate, such as sodium carbonate, sodium bicarbonate, potassium carbonate, potassium bicarbonate, calcium carbonate, calcium bicarbonate, magnesium carbonate, magnesium bicarbonate, lithium carbonate, and lithium bicarbonate.

There are two key considerations that link HCO₃⁻ as a buffer to CFTR. First, recent findings indicate that, although the relative ratio of HCO₃⁻ conductance/Cl⁻ conductance is between 0.1 and 0.2 for single CFTR channels activated with cAMP and ATP, the ratio in the sweat duct can range from virtually 0 to almost 1.0, depending on conditions of stimulation. That is, combining cAMP+cGMP+α-ketoglutarate can yield CFTR HCO₃⁻ conductance almost equal to that of Cl⁻ conductance (Quiton et al. Physiology, 22(3):212-225 (2007)). Therefore, CFTR conducts HCO₃⁻, and hence CF airway surfaces may be HCO₃⁻ depleted, or acidic, and in need of replacement therapy. Second, absent CFTR-dependent bicarbonate secretion can lead to chronic airway surface acidification and impaired capacity of CF airways to respond to airway conditions associated with acute acidification of airway surface liquid layer e.g. gastric acid inspiration (Coakley et al., Proc Natl Acad Sci USA, 100(26):16083-8 (2003)).

Buffering Systems Used as Excipients to Prevent Decrease in Airway Surface pH Consequent to Deposition of Hyperosmolar Solution on Airway Surface

Administration of hyperosmolar agents, such as 7% HS, on the airway surface can cause a transient decrease in the pH of the airway surface liquid layer (ASL). This transient decrease in pH may cause additional irritation to the airways. Therefore, it may be beneficial to co-formulate hyperosmolar agents with buffering excipients. This approach is especially relevant for diseases associated with CFTR dysfunction, as CFTR-dependent HCO₃⁻ conductance contributes significantly to the buffering of the airway surface as described above.

The hyperosmolar agents deposited as aerosols on the airway surface cause a transepithelial efflux of water onto the airway surface. Water added to ambient ASL will rapidly equilibrate with atmospheric CO₂ gas [CO₂(g)→CO₂(l)] which will rapidly form carbonic acid [CO₂(l)+H₂O(l)→H₂CO₃(l)]. Subsequently, the carbonic acid can lower the pH of the ASL [H₂CO₃(l)→HCO₃⁻+H₃O⁺]. To maintain the pH of the ASL, bicarbonate anions can be secreted from the airway epithelial cells via CFTR.

When a hyperosmolar agent is deposited on the airway surfaces at sufficiently high rates, which can cause rapid efflux of water onto the airway surface, the rapid equilibration of CO₂ in the ASL and the subsequent ASL acidification can exceed the rate of buffering ion (HCO₃⁻) secretion from the airway epithelium. Hence, a drop in pH can occur. This phenomenon may be exacerbated in human subjects with decreased CFTR function, such as in CF or COPD patients.

Formulations of hyperosmolar agents with buffering excipients of sufficient buffering capacities can be identified, so that the acidification of the ASL is attenuated or completely prevented. Exemplary buffer systems can comprise, but are not limited to, carbonic acid/carbonate/bicarbonate-based buffers; disodium hydrogen phthalate/sodium dihydrogen orthophosphate-based buffers; tris(hydroxylmethyl)aminomethane/hydrochloric acid-based buffers; barbitone sodium/hydrochloric acid-based buffers; and any combination thereof.

Due to these data, inclusion of bicarbonate anion in the formulation of 7% or >7% hypertonic saline administered by the disclosed methods would be particularly useful. Formulations containing up to 1 to 200 mM concentrations of bicarbonate anions are of particular interest for 7% or >7% HS solutions.

Also contemplated to be useful with disclosed embodiments are chemically modified osmolytes or osmolyte precursors. Such chemical modifications involve linking to the osmolyte (or precursor) an additional chemical group which alters or enhances the effect of the osmolyte or osmolyte precursor (e.g., inhibits degradation of the osmolyte molecule). Such chemical modifications have been utilized with drugs or prodrugs and are known in the art. (See, for example, U.S. Pat. Nos. 4,479,932 and 4,540,564; Shek, E. et al., J. Med. Chem. 19:113-117 (1976); Bodor, N. et al., J. Pharm. Sci. 67:1045-1050 (1978); Bodor, N. et al., J. Med. Chem. 26:313-318 (1983); Bodor, N. et al., J. Pharm. Sci. 75:29-35 (1986), each incorporated herein by reference.

Buffering Systems Used as Excipients to Normalize the Airway Surface pH

CFTR dysfunction leading to airways surface acidification, likely due to the dependence of bicarbonate secretion on CFTR function, has been described in several respiratory diseases including CF and COPD. CF airways surface liquid (ASL) has been shown to be more acidic, compared to the ASL from healthy subjects (Coakley et al., Proc Natl Acad Sci USA, 100(26):16083-8 (2003)). Similar abnormalities may occur in COPD. For this reason, it may be beneficial to co-formulate any therapeutic agent, administered to patients suffering from low airway surface liquid pH, with a buffering reagent/excipient of sufficient strength that would normalize the airway surface liquid pH. This approach is also applicable to diseases with decreased airway surface pH due to other causes than CFTR dysfunction, e.g. inflammation and/or infection.

Buffering Systems Used as Excipients to Prevent Decrease in Airway Surface pH Following Administrations of Acidic Aerosols.

Administration of large volumes of unbuffered aerosols on the airway surface can cause a transient decrease in the pH of the airway surface liquid layer (ASL). This transient decrease in pH may cause additional irritation to the airways. Therefore, it may be beneficial to co-formulate any aerosolized drug product with buffering excipients, providing sufficient maintenance of the pH of the aerosol in the neutral range and preventing decreases in the pH of the ASL upon aerosol deposition.

The pharmaceutical formulation is aerosolized by an inhalation delivery device for transnasal delivery. The inhalation delivery device is capable of generating an aerosol having particle size suitable for effectively passing the upper respiratory airways, such as the nasal passway. For example, the aerosol particles for inhalation have a volume median diameter (VMD) from about 0.5 μm to about 2.5 μm, or about 1 μm to about 2 μm; or about 1.2 μm to about 1.6 μm. As the aerosol particles contain an active agent, e.g., hypertonic saline, and a buffering agent, the size thereof can grow as they pass through the respiratory airways during inhalation process to become more effectively deposited to the lower respiratory airways, such as lung airway surfaces. In one embodiment, the aerosol particle size can grow as much as about 50% to about 150%, or about 70% to about 130%, or about 80% to about 120%, or about 100% from the initial nasal inhalation of the aerosol to the delivery of the aerosol to the lung airway surfaces.

Sodium Channel Blockers:

Coordinated ion transport by the airway epithelia directly regulates the hydration level of the mucosal surface. Importantly, sodium absorption through the epithelial sodium channel (ENaC) provides the rate-limiting step in hydration. In human subjects with loss of function, mutation in ENaC have ‘wet’ airway surfaces and extraordinarily fast mucous clearance (see above) (Kerem et al., N. Engl. J Med. 341(3):156-62 (1999)). Conversely, increased sodium absorption through ENaC has been shown to be the underlying cause of mucous dehydration and the formation of mucous plugs in the lungs of CF patients. Furthermore, transgenic mice that overexpress ENaC in the lungs have dehydrated airway surfaces and reduced/absent mucous clearance that results in death (Hummler et al., Proc. Natl. Acad. Sci. USA 94(21):11710-5 (1997)). As predicted from clinical and experimental data, pharmacological blockade of ENaC conserves liquid on airway surfaces and increases mucus clearance (Hirsh et al., J Pharmacol. Exp. Ther. 325(1):77-88 (2008)). Particular examples include, but are not limited to:

Small Molecule Channel Blockers:

Small molecule ENaC blockers are capable of directly preventing sodium transport through the ENaC channel pore. ENaC blockers that can be administered by the disclosed methods include, but are not limited to, amiloride, benzamil, phenamil, and amiloride analogues as exemplified by U.S. Pat. No. 6,858,614, U.S. Pat. No. 6,858,615, U.S. Pat. No. 6,903,105, U.S. Pat. No. 6,995,160, U.S. Pat. No. 7,026,325, U.S. Pat. No. 7,030,117, U.S. Pat. No. 7,064,129, U.S. Pat. No. 7,186,833, U.S. Pat. No. 7,189,719, U.S. Pat. No. 7,192,958, U.S. Pat. No. 7,192,959, U.S. Pat. No. 7,241,766, U.S. Pat. No. 7,247,636, U.S. Pat. No. 7,247,637, U.S. Pat. No. 7,317,013, U.S. Pat. No. 7,332,496, U.S. Pat. No. 7,345,044, U.S. Pat. No. 7,368,447, U.S. Pat. No. 7,368,450, U.S. Pat. No. 7,368,451, U.S. Pat. No. 7,375,107, U.S. Pat. No. 7,399,766, U.S. Pat. No. 7,410,968, U.S. Pat. No. 7,820,678, U.S. Pat. No. 7,842,697, U.S. Pat. No. 7,868,010, and U.S. Pat. No. 7,875,619.

Protease Inhibitors:

ENaC proteolysis is well described to increase sodium transport through ENaC. Protease inhibitors block the activity of endogenous airway proteases, thereby preventing ENaC cleavage and activation. Protease that cleave ENaC include furin, meprin, matriptase, trypsin, channel associated proteases (CAPs), and neutrophil elastases. Protease inhibitors that can inhibit the proteolytic activity of these proteases that can be administered by the disclosed methods include, but are not limited to, camostat, prostasin, furin, aprotinin, leupeptin, and trypsin inhibitors.

Nucleic Acids and Small Interfering RNAs (siRNA):

Any suitable nucleic acid (or polynucleic acid) can be used to carry out the disclosed embodiments, including but not limited to antisense oligonucleotide, siRNA, miRNA, miRNA mimic, antagomir, ribozyme, aptamer, and decoy oligonucleotide nucleic acids. See, e.g., US Patent Application Publication No. 20100316628. In general, such nucleic acids may be from 17 or 19 nucleotides in length, up to 23, 25 or 27 nucleotides in length, or more.

Any suitable siRNA active agent can be used to carry out the disclosed embodiments. Examples include, but are not limited to, those described in U.S. Pat. No. 7,517,865 and US Patent Applications Nos. 20100215588; 20100316628; 20110008366; and 20110104255. In general, the siRNAs are from 17 or 19 nucleotides in length, up to 23, 25 or 27 nucleotides in length, or more.

Secretagogues:

Mutations in the cystic fibrosis (CF) gene result in abnormal ion transport across the respiratory epithelium (Matsui et al., Cell 95:1005-15 (1998)). Excessive absorption of sodium and the inability to secrete chloride by the airway epithelium in patients with CF drives water absorption down an osmotic gradient generated by inappropriate salt absorption, dehydrating airway mucous secretions and reducing the volume of liquid in the PCL. In COPD, cigarette smoke impairs CFTR function, thus creating an acquired phenotype similar to CF.

P2Y₂ Receptor Agonists:

Agents that that may be administered by the disclosed methods include a group of P2Y₂ agonists. Purinergic (P2Y₂) receptors are abundant on luminal surface of human bronchial epithelium (HBE) and are known to stimulate Cl⁻ secretion and inhibit Na⁺ absorption (Goralski et al., Curr. Opin. Pharmacol., 10(3):294-9 (2010)).

Native agonists of P2Y₂ receptors are susceptible to enzymatic hydrolysis in vivo by a class of extracellular enzymes called ecto-nucleotidases (Lazarowski et al., J Biol. Chem. 279(35):36855-64 (2004)) that are present on human epithelial surfaces. Consequently, these agonists have very short half-lives. Given the enzymatic degradation of native agonists as well as engineered nucleotide-based P2Y₂ agonists, ectonucleotidase inhibitors such as ebselen can be administered by the disclosed methods in order to prolong half-lives of endogenous (e.g., ATP) or exogenously delivered P2Y₂ agonists.

P2Y₂ agonists that can be administered by the disclosed methods include P2Y₂ receptor agonists such as ATP, UTP, UTP-γ-S and dinucleotide P2Y₂ receptor agonists (e.g., denufosol or diquafosol) or a pharmaceutically acceptable salt thereof. The P2Y₂ receptor agonist is typically included in an amount effective to stimulate chloride and water secretion by airway surfaces, particularly nasal airway surfaces.

Suitable P2Y₂ receptor agonists are described in, but are not limited to, U.S. Pat. No. 6,264,975, U.S. Pat. No. 5,656,256, U.S. Pat. No. 5,292,498, U.S. Pat. No. 6,348,589, U.S. Pat. No. 6,818,629, U.S. Pat. No. 6,977,246, U.S. Pat. No. 7,223,744, U.S. Pat. No. 7,531,525 and U.S. Pat. Application No. 2009/0306009 each of which is incorporated herein by reference.

Activators of Alternative Chloride Channels such as CaCCs and CIC-2 Class Channels:

CaCCs are broadly expressed in mammalian cells where they are involved in a wide range of physiological functions, including transepithelial fluid secretion, oocyte fertilization, olfactory and sensory signal transduction, smooth muscle contraction, and neuronal and cardiac excitation. Single channel analysis has suggested four or more distinct CaCC subclasses, with a wide range of reported single channel conductances from less than 2 pS in cardiac myocytes to 50 pS in airway epithelial cells.

The consequences of CaCC activation are cell type specific, for example, chloride secretion in epithelial cells, action potential generation in olfactory receptor neurons, smooth muscle contraction, and prevention of polyspermia in oocytes. Although CaCCs were functionally characterized nearly three decades ago, their molecular identity has remained unclear until recently, with potential candidates including bestrophins (BEST1-BEST4) (Sun et al., Proc. Natl. Acad. Sci. USA 99:4008-4013 (2002)) and Tsunenari et al., J Biol. Chem. 278:41114-41125 (2003), the calcium activated chloride channel ClCA family proteins (Gruber et al., Genomics 54:200-214 (1998)) and ClC3 (Huang P et al., “Regulation of human CLC-3 channels by multifunctional Ca2+/calmodulin-dependent protein kinase”, JBC 276: 20093-100 (2001)).

Three independent laboratories have identified TMEM16A, also called anoctamin 1, as a strong candidate for a CaCC (Yang Y D et al., “TMEM16A confers receptor-activated calcium-dependent chloride conductance”, Nature 455: 1210-15 (2008); Caputo A et al., “TMEM16A, a membrane protein associated with calcium-dependent chloride channel activity”, Science, 322: 590-4 (2008); Schroeder B C et al., “Expression cloning of TMEM16A as a calcium-activated chloride channel subunit”, Cell 134:1019-29 (2008)).

ClC2 is a ubiquitously expressed, inwardly rectifying chloride channel that is activated by cell swelling. Suitable alternative chloride channel activators are described in U.S. Pat. Nos. 6,015,828, 6,159,969 and 7,253,295.

Modulators of CFTR Activity:

The hereditary lethal disease CF is caused by mutations in the gene encoding CFTR protein, a cAMP activated chloride channel expressed in the airway epithelia. Various mutations in CFTR cause ion transport dysfunction by limiting the chloride ion secretion to the surface of the airway epithelium via CFTR and by dys-regulation of sodium ion absorption, leading to excessive absorption of sodium cations. These defects in ion transport result in impaired hydration of airway surface liquid layer, decrease in mucus clearance and lead to progressive loss of lung function. Recently, it has been shown that CFTR functional defects are present in cigarette smoke exposed tissue, thus implying the role of CFTR dysfunction in COPD.

Over 1500 putative mutations have been described in CFTR, which can be divided into classes according to the molecular mechanism of the genetic defect (Rowe et al., Pulm. Pharmacol. Ther., 23(4):268-78 (2010)). An understanding of the biology of each of these mutations has led to therapeutic strategies based on the particular mutation type. Class I mutations include premature termination codons (PTCs, e.g. nonsense mutations) within the coding region of CFTR. Class II CFTR mutations include F508del CFTR, the most common mutation in humans. Class III and IV CFTR mutations are characterized by full-length CFTR that reaches the cell surface but exhibit reduced ion transport activity owing to abnormal channel gating (Class III, e.g. G551D) or reduced conductivity of the ion channel pore (Class IV, e.g. R117H). Similarly, splicing mutants (Class V) and mutations within the C-terminus (Class VI) are also full length, but exhibit reduced activity owing to reduced numbers of active channels within the plasma membrane. The classification of CFTR mutants can be simplified into the therapeutically relevant groups based on the activity of agents in development.

Potentiators of cell-surface cystic fibrosis transmembrane conductance regulator CFTR mutation classes that result in dysfunctional CFTR that resides at the plasma membrane include Class III, IV, V, and VI mutations and represent potential targets for CFTR activators.

CFTR activity modulating compounds that can be administered by the disclosed methods include, but are not limited to, VX-809, VX-770, VX-661 and compounds described in US 2009/0246137 A1, US 2009/0253736 A1, US 2010/0227888 A1, U.S. Pat. No. 7,645,789, US 2009/0246820 A1, US 2009/0221597 A1, US 2010/0184739 A1, US 2010/0130547 A1, US 2010/0168094 A1, U.S. Pat. No. 7,553,855, U.S. Pat. No. 7,772,259 B2, U.S. Pat. No. 7,405,233 B2, US 2009/0203752, and U.S. Pat. No. 7,499,570.

Mucus/Mucin Modifying Agents:

Reducing Agents:

Mucin proteins are organized into high molecular weight polymers via the formation of covalent (disulfide) and non-covalent bonds. Disruption of the covalent bonds with reducing agents is a well-established method to reduce the viscoelastic properties of mucus in vitro and is predicted to minimize mucus adhesiveness and improve clearance in vivo. Reducing agents are well known to decrease mucus viscosity in vitro and commonly used as an aid to processing sputum samples (Hirsch, S. R., Zastrow, J. E., and Kory, R. C., “Sputum liquefying agents: a comparative in vitro evaluation”, J. Lab. Clin. Med. 74:346-353, 1969). Examples of reducing agents include sulfide containing molecules capable of reducing protein disulfide bonds including, but not limited to, N-acetyl cysteine, cystamine, N-acystelyn, carbocysteine, glutathione, dithiothreitol and thioredoxin containing proteins.

Administration of NAC according to the disclosed methods allows an increase in the daily pulmonary dose (to increase efficacy), while decreasing the rate of presentation (to improve tolerability). With administration of NAC via aerosol infusion, the concentration of NAC on the airway surface can be maintained, despite rapid clearance and metabolism. Thus, by the disclosed methods, the duration of action of NAC on the airway surface will be extended. Deposition of NAC on the surface of the lung according to the disclosed methods can achieve this effect at rates of 0.005 mg/min to 5.4 mg/min over extended 8 hour aerosol administration and can allow for improved efficacy of NAC.

Surfactants and Detergents:

Surfactants and detergents are spreading agents shown to decrease mucus viscoelasticity, improving mucus clearability. Examples of surfactants include DPPC, PF, palmitic acid, palmitoyl-oleoylphosphatidylglycerol, surfactant proteins (e.g. SP-A, B, or C), or may be animal derived (e.g. from cow or calf lung lavage or extracted from minced pig lung) or combinations thereof. See, e.g., U.S. Pat. Nos. 7,897,577; 5,876,970; 5,614,216; 5,100,806; and 4,312,860. Examples of surfactant products include Exosurf, Pumactant, KL-4, Venticute, Alveofact, Curosurf, Infasurf, and Survanta. Examples of detergents include, but are not limited to, Tween-80 and triton-X 100. Surfactants may be used to clear adherent secretions from the lung and/or prevent apposion of upper airway surfaces that produce obstructive sleep apnea.

Buffering Agents to Increase the Activity of Reducing Agents:

Thiol containing agents, such as N-acetylcysteine, exhibit increased reducing activity as the pH environment approaches or exceeds the pKₐ of the sulfur moiety. The pH of the airway surface is maintained at ˜7.4 and is reported be more acidic in diseased airways such as CF (Jayaraman S, Song Y, Vetrivel L, Shankar L, Verkman A S, “Noninvasive in vivo fluorescence measurement of airway-surface liquid depth, salt concentration, and pH”, J Clin. Invest., 107(3):317-24 (2001)). As the pKₐ of the NAC sulfur moiety is 9.5, NAC is only partially active in the pH environment of the lung surface. Thus, NAC administered (by the disclosed methods) in combination with sufficient amounts of a buffering agent to raise the ASL pH, will increase the therapeutic potential of NAC.

Expectorants:

Any suitable expectorant can be used, including but not limited to guaifenesin (see, e.g., U.S. Pat. No. 7,345,051).

Dnase:

Any suitable deoxyribonuclease can be used, including but not limited to Dornase Alpha (see, e.g., U.S. Pat. No. 7,482,024).

Exemplary Anti-Infective Agents

Chronic obstructive pulmonary diseases are accompanied by both acute and chronic bacterial infections. Both acute and chronic infections lead to chronic inflammation that has acute flare-ups in the form of pulmonary exacerbations. The underlying inflammation is treated with a variety of inhaled anti-inflammatory agents. For example, in cystic fibrosis the most common bacteria causing chronic infection is Pseudomonas aeruginosa (P. aeruginosa) and antibiotics that are effective against this bacteria are a major component of treatment (Flume, Am. J. Respir. Crit. Care Med. 176(10):957-69 (2007)). Also bacteria such as Staphylococcus aureus (S. aureus), Burkholderia cepacia (B. cepacia) and other gram negative organisms as well as anaerobes are isolated from respiratory secretions and people with CF may benefit from treatment of these pathogens to maintain their lung health. Anaerobic bacteria are also recognized as a feature of CF airways, sinuses in subjects with chronic sinusitis, and likely airways of subjects with COPD. Similarly, aspirations or microaspirations, especially in the elderly population and during sleep, are associated with a chemical pneumonitis, anaerobic infections and subsequent bronchiectasis. An ideal treatment of aspiration-related pneumonitis and anaerobic infection would be an immediate treatment. As such, antibiotics are used to eradicate early infections, during pulmonary exacerbations and as chronic suppressive therapy.

The primary measure of antibiotic activity is the minimum inhibitory concentration (MIC). The MIC is the lowest concentration of an antibiotic that completely inhibits the growth of a microorganism in vitro. While the MIC is a good indicator of the potency of an antibiotic, it indicates nothing about the time course of antimicrobial activity. PK parameters quantify the lung tissue level time course of an antibiotic. The three pharmacokinetic parameters that are most important for evaluating antibiotic efficacy are the peak tissue level (Cmax), the trough level (Cmin), and the Area Under the tissue concentration time Curve (AUC). While these parameters quantify the tissue level time course, they do not describe the killing activity of an antibiotic.

Integrating the PK parameters with the MIC gives us three PK/PD parameters which quantify the activity of an antibiotic: the Peak/MIC ratio, the T>MIC, and the 24 h-AUC/MIC ratio. The Peak/MIC ratio is simply the Cpmax divided by the MIC. The T>MIC (time above MIC) is the percentage of a dosage interval in which the serum level exceeds the MIC. The 24 h-AUC/MIC ratio is determined by dividing the 24-hour-AUC by the MIC. The three pharmacodynamic properties of antibiotics that best describe killing activity are time-dependence, concentration-dependence, and persistent effects. The rate of killing is determined by either the length of time necessary to kill (time-dependent), or the effect of increasing concentrations (concentration-dependent). Persistent effects include the Post-Antibiotic Effect (PAE). PAE is the persistent suppression of bacterial growth following antibiotic exposure.

Using these parameters, antibiotics can be divided into 3 categories (see Table 8):

TABLE 8
Categories of Antibodies

| | | Goal of | PK/PD |
|---|---|---|---|
| Pattern of Activity | Antibiotics | Therapy | Parameter |
| Type I | Aminoglycosides | Maximize | 24 h-AUC/MIC |
| Concentration- | Daptomycin | concentrations | Peak/MIC |
| dependent killing | Fluoroquinolones | | |
| and Prolonged | Ketolides | | |
| persistent effects | | | |
| Type II | Carbapenems | Maximize | T > MIC |
| Time-dependent | Cephalosporins | duration | |
| killing and | Erythromycin | of exposure | |
| Minimal persistent | Linezolid | | |
| effects | Penicillins | | |
| Type III | Azithromycin | Maximize | 24 h-AUC/MIC |
| Time-dependent | Clindamycin | amount | |
| killing and | Oxazolidinones | of drug | |
| Moderate to | Tetracyclines | | |
| prolonged persistent | Vancomycin | | |
| effects. | | | |

For Type I antibiotics (aminoglycosides (AG's), fluoroquinolones, daptomycin and the ketolides), the ideal dosing regimen would maximize concentration, because the higher the concentration, the more extensive and the faster is the degree of killing. Therefore, the 24 h-AUC/MIC ratio, and the Peak/MIC ratio are important predictors of antibiotic efficacy. For aminoglycosides, it is best to have a Peak/MIC ratio of at least 8-10 to prevent resistance. For fluoroquinolones vs gram negative bacteria, the optimal 24 h-AUC/MIC ratio is approximately 125. Versus gram positives, 40 appears to be optimal. However, the ideal 24 h-AUC/MIC ratio for fluoroquinolones varies widely in the literature.

Type II antibiotics (beta-lactams, cephalosporins, clindamycin, erythromcyin, carbapenems and linezolid) demonstrate the complete opposite properties. The ideal dosing regimen for these antibiotics maximizes the duration of exposure. The T>MIC is the parameter that best correlates with efficacy. For beta-lactams and erythromycin, maximum killing is seen when the time above MIC is at least 70% of the dosing interval.

Type III antibiotics (oxazolidinones, vancomycin, tetracyclines, azithromycin, clindamycin and the dalfopristin-quinupristin combination) have mixed properties, they have time-dependent killing and moderate persistent effects. The ideal dosing regimen for these antibiotics maximizes the amount of drug received. Therefore, the 24 h-AUC/MIC ratio is the parameter that correlates with efficacy. For vancomycin, a 24 h-AUC/MIC ratio of at least 125 is necessary.

Given the pharmacokinetic and pharmacodynamic properties for Type II and Type III antibiotics, administration by aerosol “infusion” will improve the efficacy for such agents. For example, carbapenam antibiotics are susceptible to enzymatic hydrolysis in vivo by the enzyme dehydropeptidase-I, thus leading to a short elimination half-life (less than 2 hr). The best measure of efficacy of this class of antibiotics is based on the minimum percentage of time the drug concentration is above the minimum inhibitory concentration (MIC) in the target tissue. Most dose regimens target a time above the MIC (TaM) of at least 50%, thus the need for a continuous infusion. High systemic concentrations of carbapenems can have proconvulsive effects and renal and liver toxicity.

Delivering carbapenems via continuous aerosol to the lungs of patients in need can allow for a safe and convenient way to maintain a high TaM in the lungs while reducing potential for systemic side effects. 500 mg to 2,000 mg of inhaled meropenem administered BID in 4 ml of normal saline via Pari LC jet nebulizers may be used for treatment of CF bacterial infections. Such administrations occur at a rate of 6.7 mg/min to 26.7 mg/min of meropenem deposited in the airway surface during two 15 minute nebulization periods per day. A 20 mg to 1,200 mg dose of meropenem, deposited in the lung of CF patients per day and administered at a rate between 0.04 mg/min to 2.5 mg/min of meropenem deposited in the airway surface during 8 hour or longer extended aerosol administration according to the disclosed methods, can allow for better combined safety, tolerability and efficacy outcomes. Patients including, but not limited to, CF, COPD, non-CF bronchiectasis, aspiration pneumonia, asthma and VAP patients suffering from respiratory infection caused by bacteria susceptible to meropenem may benefit from such treatment. Examples of carbapenam antibiotics are: imipenam, panipenam, meropenam, doripenem, biapenam, MK-826, DA-1131, ER-35786, lenapenam, S-4661, CS-834 (prodrug of R-95867), KR-21056 (prodrug of KR-21012), L-084 (prodrug of LJC 11036) and CXA-101.

Exemplary Anti-Inflammatory Agents

Inhaled corticosteroids are the standard of chronic care for asthma, COPD and other respiratory diseases characterized by acute and chronic inflammation leading to airflow limitation. Examples of corticosteroids suitable for administration by the disclosed methods include, but are not limited to, beclomethasone, budesonide, and fluticasone. NSAIDs are a group of anti-inflammatory medications that do not contain steroids. NSAIDs do not carry the same risk of side effects as steroidal anti-inflammatory medications, but with long-term use, they may cause internal bleeding or kidney problems.

Products of arachidonic acid metabolism, specifically the leukotrienes (LTs), contribute to pulmonary inflammation. Cysteinylleukotrienes (LTC4, LTD4, and LTE4) are produced predominantly by eosinophils, mast cells, and macrophages. Examples of leukotriene modifiers suitable for administration by the disclosed methods include, but are not limited to, monteleukastzileuton and zafirlukast.

Mast cell stabilizers are cromone medications such as cromolyn (sodium cromoglycate) used to prevent or control certain allergic disorders. They block a calcium channel essential for mast cell degranulation, stabilizing the cell and thereby preventing the release of histamine and related mediators. As inhalers they are used to treat asthma, as nasal sprays to treat hay fever (allergic rhinitis) and as eye drops for allergic conjunctivitis. Finally, in oral form they are used to treat the rare condition of mastocytosis.

PDE4 inhibitors have been shown to modulate pulmonary inflammation and used for treatment of chronic obstructive pulmonary diseases. Examples of PDE4 inhibitors suitable for administration by the disclosed methods include, but are not limited to, theophylline and roflumilast.

Exemplary Bronchodilators

NO, NO Donors, NO and Peroxynitrite Scavengers and Inducible NO Synthase Activity Modulators:

Nitric oxide (NO) is a potent endogenous vasodilator and bronchodilator that can be exogenously administered via inhalation. It is synthesized by the conversion of the terminal guanidine nitrogen atom of L-arginine via the endothelial cell calcium dependent enzyme nitric oxide synthetase and then diffuses across the cell membrane to activate the enzyme guanylatecyclase. This enzyme enhances the synthesis of cyclic guanosine monophosphate (cGMP), causing relaxation of vascular and bronchial smooth muscle and vasodilation of blood vessels (Palmer, Circ. Res., 82(8):852-61 (1998)).

Nitric oxide synthesised in endothelial cells that line blood vessels has a wide range of functions that are vital for maintaining healthy respiratory and cardiovascular systems (Megson, I L et al., Expert Opin. Investig. Drugs 11(5):587-601 (2002)). Reduced nitric oxide availability is implicated in the initiation and progression of many diseases and delivery of supplementary nitric oxide to help prevent disease progression is an attractive therapeutic option. Nitric oxide donor drugs represent a useful means of systemic nitric oxide delivery and organic nitrates have been used for many years as effective therapies for symptomatic relief from angina. However, nitrates have limitations and a number of alternative nitric oxide donor classes have emerged since the discovery that nitric oxide is a crucial biological mediator.

Examples of NO, NO donors and NO synthase activity modulators suitable for administration by the disclosed methods include inhaled NO, inhaled NaNO₂, agents disclosed in Vallance et al., Fundam. Clin. Pharmacol., 17(1):1-10 (2003), Al-Sa'doni H H et al., Mini Rev. Med. Chem., 5(3):247-54 (2005), Miller M R et al., Br. J Pharmacol., 151(3):305-21 (2007). Epub 2007 Apr. 2 and Katsumi H et al. Cardiovasc. Hematol. Agents Med. Chem., 5(3):204-8 (2007).

Under certain conditions, inducible NO synthase activity leads to overproduction of NO which in turn increases inflammation and tissue injury. Under these conditions, the following inducible NO synthase inhibitors, NO scavengers and peroxynitrite scavengers administered by the disclosed methods are suitable: Bonnefous et al., J. Med. Chem., 52 (9):3047-3062 (2009), Muscara et al AJP-GI 276 (6):G1313-G1316 (1999) or Hansel et al. FASEB Journal, 17:1298-1300 (2003).

Beta 2-Adrenergic Receptor Agonists:

It has been established that administration of super-therapeutic concentrations of receptor agonists leads to receptor desensitization and loss of efficacy. For example, this phenomenon has been described for beta 2-adrenoceptor based bronchodilator agents (Duringer et al., Br. J Pharmacol., 158(1):169-79 (2009)). High concentration of these receptor agonist agents leads to the receptor phosphorylation, internalization and potential degradation. Administration of receptor agonists, which cause tachyphylaxis following bolus administration via fast nebulizer, by inhalation over the course of 8 to 24 hours or overnight to a patient via nasal cannula improves the efficacy of such agents due to decreased extent of tachyphylaxis. Beta 2-adrenergic receptor agonists include, but are not limited to, albuterol, levalbuterol, salbutamol, procaterol, terbutaline, pirbuterol, and metaproterenol.

Other Exemplary Therapeutic Agents

Examples of other classes of therapeutic agents suitable for administration by the disclosed methods include antivirals such as ribavirin, anti-fungal agents such as amphotericin, intraconazol and voriconazol, immunosuppressants, anti-rejection drugs such as cyclosporine, tacrolimus and sirolimus, bronchodilators including but not limited to anticholinergic agents such as ipratropium, tiotropium, aclidinium and others, PDE5 inhibitors, gene therapy vectors, aptamers, endothelin-receptor antagonists, alpha-1-antitrypsin, prostacyclins, vaccines, PDE-4 and PDE-5 inhibitors and steroids such as beclamethasone, budesonide, ciclesonide, flunisolide, fluticasone, memetasone and triamcinolone.

### Drug Classes Suitable for Targeting Extra-Pulmonary Tissues

It is well recognized that pulmonary drug delivery is an alternative means to target extra-pulmonary tissues. Systemic administration of therapeutic agents by any of the nasal cannula assemblies described herein, is useful only in cases when such therapy can be formulated in a manner that allows reaching therapeutically effective levels in the extra-pulmonary tissues of interest.

Pulmonary administration can by-pass issues associated with oral, transdermal, sublingual, IV, i.m., i.p and other injectable drug administration. For example, injection or IV administration cause pain and subjects the individual to infections at the injection site. Furthermore, aerosol delivery of drugs is advantageous to oral administration particularly for therapeutic agents that are poorly orally available or inactivated by first-pass metabolism.

The administration of therapeutic agents intended to target non-pulmonary issues by the disclosed methods is advantageous in that it can (1) overcome the limitations of oral or IV administration; and (2) it can be utilized to control the pharmacokinetic profile (e.g. blood levels over time) of the therapeutic agent.

An example of therapies particularly suitable for administration by the tPAD-based device platform include inhaled insulin for diabetes, inhaled dihydroergotamine for acute migraine, inhaled morphine for palliative care, and sleep agents for dyspnea.

An example of current IV therapies suitable for tPAD-based device administration include inotropic treatments for chronic congestive heart failure [Amrinone (Inocor®), Digitoxin (Crystodigin®), Digoxin (Lanoxin®, Lanoxicaps®), Dobutamine (Dobutrex®) or Milrinone (Primacor®)] and others.

### All Other Drug Classes Suitable for Local or Systemic Administration

Other drug classes and agents suitable for administration via any of the nasal cannula assemblies described herein for local or systemic administration include but are not limited to 5-alpha-reductase inhibitors, 5-aminosalicylates, 5HT3 receptor antagonists, adamantane antivirals, adrenal cortical steroids, adrenal corticosteroid inhibitors, adrenergic bronchodilators, agents for hypertensive emergencies, agents for pulmonary hypertension, aldosterone receptor antagonists, alkylating agents, alpha-glucosidase inhibitors, alternative medicines, amebicides, aminoglycosides, aminopenicillins, aminosalicylates, amylin analogs, analgesic combinations, analgesics, androgens and anabolic steroids, angiotensin converting enzyme inhibitors, angiotensin II inhibitors, anorectal preparations, anorexiants, antacids, anthelmintics, anti-angiogenic ophthalmic agents, anti-CTLA-4 monoclonal antibodies, anti-infectives, antiadrenergic agents, centrally acting antiadrenergic agents, peripherally acting antiandrogens, antianginal agents, antiarrhythmic agents, antiasthmatic combinations, antibiotics/antineoplastics, anticholinergic antiemetics, anticholinergic antiparkinson agents, anticholinergic bronchodilators, anticholinergic chronotropic agents, anticholinergics/antispasmodics, anticoagulants, anticonvulsants, antidepressants, antidiabetic agents, antidiabetic combinations, antidiarrheals, antidiuretic hormones, antidotes, antiemetic/antivertigo agents, antifungals, antigonadotropic agents, antigout agents, antihistamines, antihyperlipidemic agents, antihyperlipidemic combinations, antihypertensive combinations, antihyperuricemic agents, antimalarial agents, antimalarial combinations, antimalarial quinolines, antimetabolites, antimigraine agents, antineoplastic detoxifying agents, antineoplastic interferons, antineoplastics, antiparkinson agents, antiplatelet agents, antipseudomonal penicillins, antipsoriatics, antipsychotics, antirheumatics, antiseptic and germicides, antithyroid agents, antitoxins and antivenins, antituberculosis agents, antituberculosis combinations, antitussives, antiviral agents, antiviral combinations, antiviral interferons, anxiolytics, sedatives, hypnotics, aromatase inhibitors, atypical antipsychotics, azole antifungals, bacterial vaccines, barbiturate anticonvulsants, barbiturates, BCR-ABL tyrosine kinase inhibitors, benzodiazepine anticonvulsants, benzodiazepines, beta-adrenergic blocking agents, beta-lactamase inhibitors, bile acid sequestrants, biologicals, bisphosphonates, bone resorption inhibitors, bronchodilator combinations, bronchodilators, calcineurin inhibitors, calcitonin, calcium channel blocking agents, carbamate anticonvulsants, carbapenems, carbonic anhydrase inhibitor anticonvulsants, carbonic anhydrase inhibitors, cardiac stressing agents, cardioselective beta blockers, cardiovascular agents, catecholamines, CD20 monoclonal antibodies, CD30 monoclonal antibodies, CD33 monoclonal antibodies, CD52 monoclonal antibodies, central nervous system agents, cephalosporins, cerumenolytics, CFTR potentiators and correctors, chelating agents, chemokine receptor antagonist, chloride channel activators, cholesterol absorption inhibitors, cholinergic agonists, cholinergic muscle stimulants, cholinesterase inhibitors, CNS stimulants, coagulation modifiers, colony stimulating factors, contraceptives, corticotropin, coumarins and indandiones, cox-2 inhibitors, decongestants, dermatological agents, diagnostic radiopharmaceuticals, dibenzazepine anticonvulsants, digestive enzymes, dipeptidyl peptidase 4 inhibitors, diuretics, dopaminergic antiparkinsonism agents, drugs used in alcohol dependence, echinocandins, EGFR inhibitors, estrogen receptor antagonists, estrogens, expectorants, factor Xa inhibitors, fatty acid derivative anticonvulsants, fibric acid derivatives, first generation cephalosporins, fourth generation cephalosporins, functional bowel disorder agents, gallstone solubilizing agents, gamma-aminobutyric acid analogs, gamma-aminobutyric acid reuptake inhibitors, gastrointestinal agents, general anesthetics, genitourinary tract agents, GI stimulants, glucocorticoids, glucose elevating agents, glycopeptide antibiotics, glycoprotein platelet inhibitors, glycylcyclines, gonadotropin releasing hormones, gonadotropin-releasing hormone antagonists, gonadotropins, group I antiarrhythmics, group II antiarrhythmics, group III antiarrhythmics, group IV antiarrhythmics, group V antiarrhythmics, growth hormone receptor blockers, growth hormones, H. pylori eradication agents, H2 antagonists, hedgehog pathway inhibitors, hematopoietic stem cell mobilizers, heparin antagonists, heparins, HER2 inhibitors, herbal products, histone deacetylase inhibitors, hormones, hormones/antineoplastics, hydantoin anticonvulsants, immune globulins, immunologic agents, immunostimulants, immunosuppressive agents, impotence agents, in vivo diagnostic biologicals, incretin mimetics, inhaled anti-infectives, inhaled corticosteroids, inotropic agents, insulin, insulin-like growth factor, integrase strand transfer inhibitors, interferons, interleukin inhibitors, interleukins, intravenous nutritional products, iodinated contrast media, ionic iodinated contrast media, iron products, ketolides, laxatives, leprostatics, leukotriene modifiers, lincomycin derivatives, local injectable anesthetics, loop diuretics, lung surfactants, lymphatic staining agents, lysosomal enzymes, macrolide derivatives, macrolides, magnetic resonance imaging contrast media, mast cell stabilizers, medical gas, meglitinides, metabolic agents, methylxanthines, mineralocorticoids, minerals and electrolytes, miscellaneous analgesics, miscellaneous antibiotics, miscellaneous anticonvulsants, miscellaneous antidepressants, miscellaneous antidiabetic agents, miscellaneous antiemetics, miscellaneous antifungals, miscellaneous antihyperlipidemic agents, miscellaneous antimalarials, miscellaneous antineoplastics, miscellaneous antiparkinson agents, miscellaneous antipsychotic agents, miscellaneous antituberculosis agents, miscellaneous antivirals, miscellaneous anxiolytics, sedatives and hypnotics, miscellaneous bone resorption inhibitors, miscellaneous cardiovascular agents, miscellaneous central nervous system agents, miscellaneous coagulation modifiers, miscellaneous diuretics, miscellaneous genitourinary tract agents, miscellaneous GI agents, miscellaneous hormones, miscellaneous metabolic agents, miscellaneous ophthalmic agents, miscellaneous otic agents, miscellaneous respiratory agents, miscellaneous sex hormones, miscellaneous vaginal agents, mitotic inhibitors, monoamine oxidase inhibitors, mouth and throat products, mTOR inhibitors, mucolytics, multikinase inhibitors, muscle relaxants, mydriatics, narcotic analgesic combinations, narcotic analgesics, nasal anti-infectives, nasal antihistamines and decongestants, nasal lubricants and irrigations, nasal preparations, nasal steroids, natural penicillins, neuraminidase inhibitors, neuromuscular blocking agents, neuronal potassium channel openers, next generation cephalosporins, nicotinic acid derivatives, NNRTIs, non-cardioselective beta blockers, non-iodinated contrast media, non-ionic iodinated contrast media, non-sulfonylureas, nonsteroidal anti-inflammatory agents, nucleoside reverse transcriptase inhibitors (NRTIs), nutraceutical products, nutritional products, ophthalmic anesthetics, ophthalmic anti-infectives, ophthalmic anti-inflammatory agents, ophthalmic antihistamines and decongestants, ophthalmic glaucoma agents, ophthalmic steroids, ophthalmic steroids with anti-infectives, oral nutritional supplements, other immunostimulants, other immunosuppressants, otic anesthetics, otic anti-infectives, otic preparations, otic steroids, otic steroids with anti-infectives, oxazolidinedione anticonvulsants, parathyroid hormone and analogs, penicillinase resistant penicillins, penicillins, peripheral opioid receptor antagonists, peripheral vasodilators, peripherally acting antiobesity agents, phenothiazine antiemetics, phenothiazine antipsychotics, phenylpiperazine antidepressants, plasma expanders, platelet aggregation inhibitors, platelet-stimulating agents, polyenes, potassium-sparing diuretics, probiotics, progesterone receptor modulators, progestins, prolactin inhibitors, prostaglandin D2 antagonists, protease inhibitors, proton pump inhibitors, psoralens, psychotherapeutic agents, psychotherapeutic combinations, purine nucleosides, pyrrolidine anticonvulsants, quinolones, radiocontrast agents, radiologic adjuncts, radiologic agents, radiologic conjugating agents, radiopharmaceuticals, recombinant human erythropoietins, renin inhibitors, chemotherapies, rifamycin derivatives, salicylates, sclerosing agents, second generation cephalosporins, selective estrogen receptor modulators, selective immunosuppressants, selective phosphodiesterase-4 inhibitors, selective serotonin reuptake inhibitors, serotonin-norepinephrine reuptake inhibitors, serotoninergic neuroenteric modulators, sex hormone combinations, sex hormones, skeletal muscle relaxant combinations, skeletal muscle relaxants, smoking cessation agents, somatostatin and somatostatin analogs, spermicides, statins, sterile irrigating solutions, streptomyces derivatives, succinimide anticonvulsants, sulfonamides, sulfonylureas, synthetic ovulation stimulants, tetracyclic antidepressants, tetracyclines, therapeutic radiopharmaceuticals, therapeutic vaccines, thiazide diuretics, thiazolidinediones, thioxanthenes, third generation cephalosporins, thrombin inhibitors, thrombolytics, thyroid drugs, TNF alfa inhibitors, tocolytic agents, anesthetics, anti-infectives, antibiotics, antifungals, antihistamines, antineoplastics, antipsoriatics, antivirals, astringents, debriding agents, depigmenting agents, non-steroidal anti-inflammatories, photochemotherapeutics, chemotherapies, rubefacient, steroids, steroids with anti-infectives, triazine anticonvulsants, tricyclic antidepressants, trifunctional monoclonal antibodies, ultrasound contrast media, upper respiratory combinations, urea anticonvulsants, urinary anti-infectives, urinary antispasmodics, urinary pH modifiers, uterotonic agents, vaccine combinations, vaginal anti-infectives, vaginal preparations, vasodilators, vasopressin antagonists, vasopressors, VEGF/VEGFR inhibitors, viral vaccines, vitamin and mineral combinations, and vitamins.

### List of Diseases and Conditions Treated by Therapies Administered by Nasal Cannula Assemblies

Diseases and conditions that are treatable by therapies administered by the nasal cannula assemblies described herein include but are not limited to, Abdominal Aortic Aneurysm, Acanthamoeba Infection, Acinetobacter Infection, Acquired Immunodeficiency Syndrome (AIDS), Adenovirus Infection, ADHD [Attention Deficit/Hyperactivity Disorder], African Trypanosomiasis, ALS [Amyotrophic Lateral Sclerosis], Alzheimer's Disease, Amebiasis; Intestinal [Entamoeba histolytica infection], American Trypanosomiasis, Amphibians and Fish; Infections from, Amyotrophic Lateral Sclerosis, Anaplasmosis; Human, Anemia, Angiostrongylus Infection, Animal-Related Diseases, Anisakis Infection [Anisakiasis], Anthrax, Antibiotic and Antimicrobial Resistance, Aortic Aneurysm, Arenavirus Infection, Arthritis, Childhood Arthritis, Fibromyalgia, Gout, Lupus (SLE) [Systemic lupus erythematosus], Osteoarthritis (OA), Rheumatoid Arthritis (RA), Ascaris Infection [Ascariasis], ASDs (Autism), Aseptic Meningitis, Aspergillus Infection [Aspergillosis], Asthma, Autism, autism spectrum disorders, Avian Influenza, B virus Infection [Herpes B virus], B. cepacia infection (Burkholderia cepacia Infection), Babesiosis [Babesia Infection], Bacterial Meningitis, Bacterial Vaginosis (BV), Balamuthia infection [Balamuthia mandrillaris infection], Balantidium Infection [Balantidiasis], Baylisascaris Infection, Bilharzia, Bioterrorism Agents/Diseases, Bird Flu, Birth Defects, Black Lung [Coal Workers' Pneumoconioses], Blastocystis Infection [Blastocystis hominis Infection], Blastomycosis, Bleeding Disorders, Blood Disorders, Body Lice [Pediculus humanus corporis], Bone Health, Borrelia burgdorferi Infection (Lyme Disease), Botulism [Clostridium botulinim], Bovine Spongiform Encephalopathy (BSE), Brainerd Diarrhea, Breast and Ovarian Cancer, Bronchitis, Brucella Infection [Brucellosis], BSE (Bovine Spongiform Encephalopathy), Burkholderia cepacia Infection (B. cepacia infection), Burkholderia mallei, Burkholderia pseudomallei Infection, BV (Bacterial Vaginosis), Campylobacter Infection [Campylobacteriosis], Cancer, Colorectal (Colon) Cancer, Gynecologic Cancers, Lung Cancer, Prostate Cancer, Skin Cancer, Candida Infection [Candidiasis], Canine Flu, Capillaria Infection [Capillariasis], Carbapenem resistant Klebsiella pneumonia (CRKP), Carpal Tunnel Syndrome, Cat Flea Tapeworm, Cats; Infections from, Cercarial Dermatitis, Cerebral Palsy, Cervical Cancer, CFS (Chronic Fatigue Syndrome), Chagas Disease [Trypanosoma cruzi Infection], Chest Cold, Chickenpox [Varicella Disease], Chikungunya Fever (CHIKV), Childhood Arthritis, Childhood Diseases, German Measles [Rubella Virus], Measles, Mumps, Rotavirus Infection, Children's Cough, Chlamydia [Chlamydia trachomatis Disease], Chlamydia pneumoniae Infection, Cholera [Vibrio cholerae Infection], Chronic Fatigue Syndrome (CFS), Chronic Obstructive Pulmonary Disease (COPD), Ciguatera Fish Poisoning, Classic Creutzfeldt-Jakob Disease, Clonorchis Infection [Clonorchiasis], Clostridium botulinim, Clostridium difficile Infection, Clostridium perfringens infection, Clostridium tetani Infection, Clotting Disorders, CMV (Cytomegalovirus Infection), Coal Workers' Pneumoconioses, Coccidioidomycosis, Cold; Common, Colorectal (Colon) Cancer, Concussion, Congenital Hearing Loss, Conjunctivitis, Cooleys Anemia, COPD (Chronic Obstructive Pulmonary Disease), Corynebacterium diphtheriae Infection, Coxiella burnetii Infection, CRKP (Carbapenem resistant Klebsiella pneumonia), Crohn's Disease, Cryptococcosis, Cryptosporidium Infection [Cryptosporidiosis], Cyclospora Infection [Cyclosporiasis], Cysticercosis, Cystoisospora Infection [Cystoisosporaiasis], Cytomegalovirus Infection (CMV), DBA (Diamond Blackfan Anemia), Dengue Fever (DF), Dengue Hemorrhagic Fever (DHF), Dermatophytes, Dermopathy; Unexplained, Diabetes, Diamond Blackfan Anemia (DBA), Dientamoeba fragilis Infection, Diphtheria [Corynebacterium diphtheriae Infection], Diphyllobothrium Infection [Diphyllobothriasis], Dipylidium Infection, Dog Bites, Dog Flea Tapeworm, Dogs; Infections from, Down Syndrome [Trisomy 21], Dracunculiasis, Dwarf Tapeworm [Hymenolepis Infection], E. coli Infection [Escherichia coli Infection], Ear Infection [Otitis Media], Eastern Equine Encephalitis (EEE), Ebola Hemorrhagic Fever, EBV Infection (Epstein-Barr Virus Infection), Echinococcosis, EEE (Eastern Equine Encephalitis), Ehrlichiosis; Human, Elephantiasis, Emerging Infectious Diseases, Encephalitis; Mosquito-Borne and Tick-Borne, Entamoeba histolytica infection, Enterobius vermicularis Infection, Enterovirus Infections (Non-Polio), Epidemic Typhus, Epilepsy, Epstein-Barr Virus Infection (EBV Infection), Ergonomic and Musculoskeletal Disorders, Extensively Drug-Resistant TB (XDR TB), Extreme Cold [Hypothermia], Extreme Heat [Hyperthermia], Farm Animals; Infections from, Fasciitis; Necrotizing, Fasciola Infection [Fascioliasis], Fasciolopsis Infection [Fasciolopsiasis], Fetal Alcohol Syndrome, Fibromyalgia, Fifth Disease [Parvovirus B19 Infection], Filariasis; Lymphatic, Fish and Amphibians; Infections from, Flavorings-Related Lung Disease, Flu; Pandemic, Flu; Seasonal, Folliculitis, Food-Related Diseases, Clostridium perfringens infection, Fragile X Syndrome, Francisella tularensis Infection, GAE (Granulomatous amebic encephalitis), GAS (Group A Strep Infection), Gastroenteritis; viral, GBS (Group B Strep Infection), Genital Candidiasis [Vulvovaginal Candidiasis (VVC)], Genital Herpes [Herpes Simplex Virus Infection], Genital Warts—Human Papillomavirus Infection, German Measles [Rubella Virus], Giardia Infection [Giardiasis], Glanders [Burkholderia mallei], Gnathostomiasis [Gnathostoma Infection], Gonorrhea [Neisseria gonorrhoeae Infection], Gout, Granulomatous amebic encephalitis (GAE), Group A Strep Infection (GAS) [Group A Streptococcal Infection], Group B Strep Infection (GBS) [Group B Streptococcal Infection], Guillain-Barré Syndrome, Guinea Worm Disease [Dracunculiasis], Gynecologic Cancers, Cervical Cancer, Ovarian Cancer, Uterine Cancer, Vaginal and Vulvar Cancers, H1N1 Flu, H5N1, Haemophilus influenzae Infection (Hib Infection), Hand, Foot, and Mouth Disease (HFMD), Hansen's Disease, Hantavirus Pulmonary Syndrome (HPS), Head Lice [Pediculus humanus capitis], Healthcare Associated Infections, Hearing Loss in Children, Heart Disease [Cardiovascular Health], Heat Stress, Hemochromatosis, Hemophilia, Hemorrhagic Fevers (VHF); Viral, Hendra Virus Infection, Hepatitis; Viral, Hereditary Bleeding Disorders, Herpes B virus, Herpes Simplex Virus Infection, Herpes Zoster, Herpes; Genital, Herpesvirus B, Herpesvirus simiae, Heterophyes Infection [Heterophyiasis], HFMD (Hand, Foot, and Mouth Disease), Hib, Hib Infection (Haemophilus influenzae Infection), High Blood Pressure, Histoplasmosis [Histoplasma capsulatum Disease], HIV/AIDS, HIV/AIDS and STDs, Hookworm; Zoonotic, Horses; Infections from, Hot Tub Rash [Pseudomonas dermatitis Infection], HPS (Hantavirus Pulmonary Syndrome), HPV Infection (Human Papillomavirus Infection), HPV-Associated Cancers, Human Ehrlichiosis, Human Immunodeficiency Virus, Hymenolepis Infection, Hypertension, Hyperthermia, Hypothermia, IBD (Inflammatory Bowel Disease), Impetigo, Infectious Mononucleosis, Infertility, Inflammatory Bowel Disease (IBD), Influenza, Avian Influenza, Pandemic Flu, Seasonal Flu, Swine Influenza, Influenza; Avian, Influenza; Pandemic, Insects and Arthropod-Related Diseases, Intestinal Amebae Infection; Nonpathogenic, Invasive Candidiasis, Iron Deficiency, Iron Overload [Hemochromatosis], Isospora Infection [Isosporiasis], Japanese Encephalitis, Jaundice, K. pneumoniae (Klebsiella pneumoniae), Kala-Azar, Kawasaki Syndrome (KS), Kernicterus, Klebsiella pneumoniae (K. pneumoniae), La Crosse Encephalitis (LAC), La Crosse Encephalitis virus (LACV)—see La Crosse Encephalitis, Lassa Fever, Latex Allergies, LCMV (Lymphocytic Choriomeningitis), Lead Poisoning, Legionellosis, Legionnaires' Disease [Legionellosis], Leishmania Infection [Leishmaniasis], Leprosy, Leptospira Infection [Leptospirosis], Leukemia, LGV (Lymphogranuloma venereum Infection), Listeria Infection [Listeriosis], Liver Disease and Hepatitis, Loiasis [Loa boa Infection], Lou Gehrig's Disease, Lung Cancer, Lupus (SLE) [Systemic lupus erythematosus], Lyme Disease [Borrelia burgdorferi Infection], Lymphatic Filariasis, Lymphedema, Lymphocytic Choriomeningitis (LCMV), Lymphogranuloma venereum Infection (LGV), MAC (Mycobacterium avium Complex), Mad Cow Disease (BSE), Malaria, Marburg Hemorrhagic Fever, Marine Toxins, MDR TB (Multidrug-Resistant TB), Measles, Melioidosis [Burkholderia pseudomallei Infection], Meningitis [Meningococcal Disease], Menopause, Mental Retardation, Methicillin Resistant Staphylococcus aureus (MRSA), Micronutrient Malnutrition, Microsporidia Infection, Molluscum Contagiosum, Monkey B virus, Monkeypox, Mononucleosis; Infectious, Morgellons, Mosquito-Borne Diseases, Motor Vehicle Injuries, MRSA (Methicillin Resistant Staphylococcus aureus), Mucormycosis, Multidrug-Resistant TB (MDR TB), Mumps, Musculoskeletal Disorders, Mycobacterium abscessus Infection, Mycobacterium avium Complex (MAC), Mycobacterium tuberculosis Infection, Mycoplasma pneumoniae Infection, Myelomeningocele, Myiasis, Naegleria Infection [Primary Amebic Meningoencephalitis (PAM)], Necrotizing Fasciitis, Neglected Tropical Diseases (NTD), Neisseria gonorrhoeae Infection, Neurocysticercosis, New Variant Creutzfeldt-Jakob Disease, Newborn Jaundice [Kernicterus], Nipah Virus Encephalitis, Nocardiosis, Non-Polio Enterovirus Infections, Nonpathogenic (Harmless) Intestinal Protozoa, Norovirus Infection, Norwalk-like Viruses (NLV), Novel H1N1 Flu, NTD (Neglected Tropical Diseases), OA (Osteoarthritis), Obesity, Occupational Cancers, Occupational Skin Conditions, Occupational Stress, Onchocerciasis, Opisthorchis Infection, Oral Cancer, Orf Virus, Oropharyngeal Candidiasis (OPC), Osteoarthritis (OA), Osteoporosis, Otitis Media, Ovarian Cancer, PAD (Peripheral Arterial Disease), Pandemic Flu, Paragonimiasis, Paragonimus Infection [Paragonimiasis], Parasitic Diseases, Parvovirus B19 Infection, Pelvic Inflammatory Disease (PID), Peripheral Arterial Disease (PAD), Peripheral Arterial Insufficiency, Peripheral Arterial Occlusive Disease, Peripheral Vascular Disease, Pertussis, Pet-Related Diseases, PID (Pelvic Inflammatory Disease), Pink Eye [Conjunctivitis], Pinworm Infection [Enterobius vermicularis Infection], Plague [Yersinia pestis Infection], Pneumoconioses; Coal Workers', Pneumocystis carinii Pneumonia (PCP) Infection, Pneumocystis jirovecii Pneumonia, Pneumonia, Polio Infection [Poliomyelitis Infection], Poliomyelitis Infection, Pontiac Fever, Primary Amebic Meningoencephalitis (PAM), Primary Ciliary Dyskinesia, Prion Diseases [Transmissible spongiform encephalopathies (TSEs)], Prostate Cancer, Pseudomonas dermatitis Infection, Psittacosis, Pulmonary Hypertension, Q Fever [Coxiella burnetii Infection], RA (Rheumatoid Arthritis), Rabies, Raccoon Roundworm Infection [Baylisascaris Infection], Rat-Bite Fever (RBF) [Streptobacillus moniliformis Infection], Recreational Water Illness (RWI), Relapsing Fever, Reptiles; Infections from, Respiratory Syncytial Virus Infection (RSV), Rheumatoid Arthritis (RA), Rickettsia rickettsii Infection, Rickettsia; Spotted Fever Group, Rickettsial Diseases, Rift Valley Fever (RVF), Ringworm [Dermatophytes], River Blindness [Onchocerciasis], RMSF (Rocky Mountain Spotted Fever), Rodents; Diseases from, Rotavirus Infection, RSV (Respiratory Syncytial Virus Infection), Rubella Virus, Rubeola, Runny Nose, RVF (Rift Valley Fever), Salmonella typhi Infection, Salmonella Infection [Salmonellosis], SARS [Severe Acute Respiratory Syndrome], Scabies, Scarlet Fever, Schistosoma Infection, Schistosomiasis Seasonal Flu, Severe Acute Respiratory Syndrome, Sexually Transmitted Diseases (STDs), Bacterial Vaginosis (BV), Chlamydia [Chlamydia trachomatis Disease], Genital Herpes [Herpes Simplex Virus Infection], Gonorrhea [Neisseria gonorrhoeae Infection], Human Papillomavirus Infection (HPV Infection), Syphilis [Treponema pallidum Infection], SFGR (Spotted Fever Group Rickettsia), Shellfish-Associated Foodborne Illnesses, Shigella Infection [Shigellosis], Shingles [Varicella Zoster Virus (VZV)], Sickle Cell Disease, SIDS (Sudden Infant Death Syndrome), Sinus Infection [Sinusitus], Skin Cancer, Skin Conditions; Occupational, SLE (Lupus), Sleep and Sleep Disorders, Sleeping Sickness [African Trypanosomiasis], Smallpox [Variola Major and Variola Minor], Sore Mouth Infection [Orf Virus], Sore Throat, Southern Tick-Associated Rash Illness (STARI), Spina Bifida [Myelomeningocele], Spirillum minus Infection, Sporotrichosis, Spotted Fever Group Rickettsia (SFGR), St. Louis Encephalitis, Staph, Staphylococcus aureus Infection, STARI (Southern Tick-Associated Rash Illness), STDs (Sexually Transmitted Diseases), Stomach Flu, Strep Infection; Group A, Strep Infection; Group B, Strep Throat, Streptobacillus moniliformis Infection, Streptococcal Diseases, Streptococcus pneumoniae Infection, Stress; Occupational, Stroke, Strongyloides Infection [Strongyloidiasis], Sudden Infant Death Syndrome (SIDS), Swimmer's Itch [Cercarial Dermatitis], Swine Flu, Swine Influenza, Symptom Relief for Upper Respiratory Infections, Syphilis [Treponema pallidum Infection], Systemic lupus erythematosus, Tapeworm Infection [Taenia Infection], Tapeworm; Dog and Cat Flea [Dipylidium Infection], TB (Tuberculosis), TB and HIV Coinfections, TB in African-Americans TBI (Traumatic Brain Injury), Testicular Cancer, Tetanus Disease [Clostridium tetani Infection], Thalassemia, Thoracic Aortic Aneurysm, Throat; Sore, Throat; Strep, Thrombophilia, Thrombosis, Thrush [Oropharyngeal Candidiasis (OPC)], Tick-borne Relapsing Fever, Tickborne Diseases, Anaplasmosis; Human, Babesiosis [Babesia Infection], Ehrlichiosis; Human, Lyme Disease [Borrelia burgdorferi Infection], Tourette Syndrome (TS), Toxic Shock Syndrome (TSS), Toxocara Infection, Toxocariasis [Toxocara Infection], Toxoplasma Infection, Toxoplasmosis Trachoma Infection, Transmissible spongiform encephalopathies (TSEs), Traumatic Brain Injury (TBI), Traumatic Occupational Injuries, Treponema pallidum Infection, Trichinellosis (Trichinosis), Trichomoniasis [Trichomonas Infection], Trichuriasis, Trisomy 2, Trypanosoma cruzi Infection, Trypanosomiasis; African, TSS (Toxic Shock Syndrome), Tuberculosis (TB) [Mycobacterium tuberculosis Infection], Tuberculosis and HIV Coinfection, Tularemia [Francisella tularensis Infection], Typhoid Fever [Salmonella typhi Infection], Typhus Fevers, Ulcerative Colitis, Undulant Fever, Unexplained Dermopathy, Unexplained Respiratory Disease Outbreaks (URDO), Upper Respiratory Infection Symptom Relief, URDO (Unexplained Respiratory Disease Outbreaks), Uterine Cancer, Vaginal and Vulvar Cancers, Vaginal Yeast Infection, Vancomycin-Intermediate/Resistant Staphylococcus aureus Infections [VISA/VRSA], Vancomycin-resistant Enterococci Infection (VRE), Variant Creutzfeldt-Jakob Disease (vCJD), Varicella Disease, Varicella Zoster Virus (VZV), Varicella-Zoster Virus Infection, Variola Major and Variola Minor, Vibrio cholerae Infection, Vibrio parahaemolyticus Infection, Vibrio vulnificus Infection, Viral Gastroenteritis, Viral Hemorrhagic Fevers (VHF), Viral Hepatitis, Viral Meningitis [Aseptic Meningitis], Vision Impairment, Von Willebrand Disease VRE (Vancomycin-resistant Enterococci Infection), Vulvovaginal Candidiasis (VVC), VZV (Varicella Zoster Virus), West Nile Virus Infection, Western Equine Encephalitis Infection, Whipworm Infection [Trichuriasis], Whitmore's Disease, Whooping Cough, Wildlife; Infections from, Women's Bleeding Disorders, XDR TB (Extensively Drug-Resistant TB), Xenotropic Murine Leukemia Virus-related Virus Infection—(XMRV Infection, Yellow Fever, Yersinia enterocolitica Infection, Yersinia pestis Infection, Yersiniosis [Yersinia enterocolitica Infection], Zoonotic Hookworm and Zygomycosis.

While various embodiments have been described above, it should be understood that they have been presented by way of example only, and not limitation. Where methods described above indicate certain events occurring in certain order, the ordering of certain events may be modified. Additionally, certain of the events may be performed concurrently in a parallel process when possible, as well as performed sequentially as described above.

Although many of the medicaments shown and described above have been described as being in liquid form, in some embodiments, any of the compositions and/or medicaments can be in a lyophilized form that is reconstituted prior to administration. Similarly stated, in some embodiments, a cartridge can include a first portion of the medicament stored as a dry component and second portion of the medicament stored as liquid diluent.

Although, delivery protrusions included in the face pieces described herein are shown and described as straight, or curved, in some embodiments, the delivery protrusions can have any other shape. For example, the delivery protrusions can be bent, have multiple bends or curves, or configured to define a helical shape.

Although various embodiments have been described as having particular features and/or combinations of components, other embodiments are possible having a combination of any features and/or components from any of embodiments where appropriate. For example, any of the face pieces described herein can be used to deliver any of the compositions and/or treat any of the conditions identified herein.

What is claimed is:

1. An apparatus comprising:
a nasal cannula assembly including a face piece, the face piece including a plenum portion and a nasal interface portion, the plenum portion configured to be fluidically coupled to a supply line, the plenum portion defining a flow path configured to receive an aerosol flow from the supply line, the nasal interface portion including a first delivery protrusion and a second delivery protrusion, the first delivery protrusion configured to convey a first portion of the aerosol flow to a first nostril, the second delivery protrusion configured to convey a second portion of the aerosol flow to a second nostril,
the plenum portion including a side wall having a curved surface configured to redirect the second portion of the aerosol flow towards the second delivery protrusion, the side wall configured to fluidically isolate the flow path from a volume downstream from the second delivery protrusion.

2. The apparatus of claim 1, wherein the side wall is configured to limit recirculation of the second portion of the aerosol flow at a location downstream of the second delivery protrusion.

3. The apparatus of claim 1, wherein the curved surface of the side wall defines an angle of curvature of less than about 90 degrees.

4. The apparatus of claim 1, wherein the second delivery protrusion defines a nasal flow path, the curved surface forming a continuous boundary between the flow path of the plenum portion and the nasal flow path.

5. The apparatus of claim 1, wherein the flow path is characterized by a first cross-sectional flow area upstream from the first delivery protrusion and a second cross-sectional flow area between the first delivery protrusion and the second delivery protrusion, the second cross-sectional flow area less than the first cross-sectional flow area.

6. The apparatus of claim 1, wherein a portion of the side wall defines a flow restriction within the flow path.

7. The apparatus of claim 1, wherein the flow path is characterized by a first cross-sectional shape at a first location upstream from the first delivery protrusion and a second cross-sectional shape at a second location downstream from the first location, the second cross-sectional shape being different than the first cross-sectional shape.

8. The apparatus of claim 1, wherein the second delivery protrusion defines a nasal flow path, a center line of the nasal flow path being curved.

9. The apparatus of claim 1, wherein the face piece includes a connection portion configured to be coupled to the supply line such that an inner surface of the supply line and an inner surface defining the flow path of the plenum portion form a substantially continuous surface.

10. An apparatus comprising:
a nasal cannula assembly including a face piece, the face piece including a plenum portion and a nasal interface portion, the plenum portion having a side wall defining a flow path, the flow path configured to receive an aerosol flow, the nasal interface portion including a first delivery protrusion and a second delivery protrusion, the first delivery protrusion configured to convey a first portion of the aerosol flow to a first nostril, the second delivery protrusion configured to convey a second portion of the aerosol flow to a second nostril,
the flow path being characterized by a first cross-sectional flow area upstream from the first delivery protrusion and a second cross-sectional flow area between the first delivery protrusion and the second delivery protrusion, the second cross-sectional flow area less than the first cross-sectional flow area.

11. The apparatus of claim 10, wherein the plenum portion includes a side wall having a curved surface defining at least in part, the second cross-sectional flow area, the curved surface configured to redirect the second portion of the aerosol flow towards the second delivery protrusion, the side wall configured to fluidically isolate the flow path from a volume downstream from the second delivery protrusion.

12. The apparatus of claim 10, wherein the plenum portion includes a side wall having a curved surface defining at least in part, the second cross-sectional flow area, the side wall configured to limit recirculation of the second portion of the aerosol flow at a location downstream of the second delivery protrusion.

13. A method, comprising:
delivering an aerosolized osmolyte to a nasal cannula assembly, the nasal cannula assembly including a supply tube and a face piece, the face piece including a plenum portion and a nasal interface portion, the nasal interface portion including a first delivery protrusion and a second delivery protrusion, the plenum portion including a side wall defining at least a portion of a flow path, the side wall configured to fluidically isolate the flow path from a volume downstream from the second delivery protrusion; and
delivering the aerosolized osmolyte from the face piece via the flow path defined by the plenum portion such that a first portion of the aerosolized osmolyte is conveyed from the first delivery protrusion and a second portion of the aerosolized osmolyte is conveyed from the second delivery protrusion.

14. The method of claim 13, wherein the delivering the aerosolized osmolyte from the face piece is performed continuously over a period of at least one hour.

15. The method of claim 13, wherein the aerosolized osmolyte includes hypertonic saline having a saline concentration of about seven percent.

16. The method of claim 13, wherein the side wall has a curved surface configured to redirect the second portion of the aerosolized osmolyte towards the second delivery protrusion.

17. The method of claim 13, wherein the flow path is characterized by a first cross-sectional flow area upstream from the first delivery protrusion and a second cross-sectional flow area between the first delivery protrusion and the second delivery protrusion, the second cross-sectional flow area less than the first cross-sectional flow area.
